SEQUENCE LISTING (13 sequences)

SEQ ID NO: 1 - DNA, 61 bp, Homo sapiens
  misc_feature (31)..(31): r at position 31 can be a or g
cagcaacaaggccatgaacctcgcgctggaracggccgctgcccagcgccacctgccagag

SEQ ID NO: 2 - DNA, 61 bp, Homo sapiens
  misc_feature (31)..(31): y at position 31 is c or t
cggggactacgtggccctgcagaccaagggygccctgcagaagaagccgctctacgtggcc

SEQ ID NO: 3 - DNA, 20 bp, Homo sapiens
acatgagaagctgggcactc

SEQ ID NO: 4 - DNA, 20 bp, Homo sapiens
cgtcacctccatcgacttct

SEQ ID NO: 5 - DNA, 19 bp, Homo sapiens
ggccgtttccacctgaatc

SEQ ID NO: 6 - DNA, 19 bp, Homo sapiens
tgatgctctgggaattcag

SEQ ID NO: 7 - DNA, 20 bp, Homo sapiens
cagctaccttcgagccattc

SEQ ID NO: 8 - DNA, 21 bp, Homo sapiens
taggctagacgtccaggaaca

SEQ ID NO: 9 - DNA, 21 bp, Homo sapiens
tattggcaggactgagtggag

SEQ ID NO: 10 - DNA, 20 bp, Homo sapiens
caaggccttctgcatgttct

SEQ ID NO: 11 - DNA, 20 bp, Homo sapiens
gtgaatgaccatgctgttgc

SEQ ID NO: 12 - DNA, 21 bp, Homo sapiens
tccttctactttgcgatcacg

SEQ ID NO: 13 - DNA, 21 bp, Homo sapiens
catggtcaagaacctgaggac

(19) United States
(12) Patent Application Publication
(10) Pub. No.: US 2011/0014607 A1
(43) Pub. Date: Jan. 20, 2011

(54) IMPRINTED GENES AND DISEASE

(76) Inventors: Randy L. Jirtle, Durham, NC (US); Alexander J. Hartemink, Durham, NC (US); Philippe P. Luedi, Basel (CH)

Correspondence Address: JENKINS, WILSON, TAYLOR & HUNT, P. A., 3100 Tower Blvd., Suite 1200, DURHAM, NC, 27707, US

(21) Appl. No.: 12/517,952

(22) PCT Filed: Dec. 6, 2007
(86) PCT No.: PCT/US07/24973

§ 371 Date: Oct. 4, 2010

Related U.S. Application Data
(60) Provisional application No. 60/873,151, filed on Dec. 6, 2006.

(51) Int. Cl. C12Q 1/68; G06F 19/00; G06F 15/18

(52) U.S. Cl. 435/6; 702/19; 706/12

(57) ABSTRACT

Methods for identifying imprinted genes. In some embodiments, the methods comprise (a) providing a first data set comprising a plurality of nucleic acid sequences, wherein the nucleic acid sequences comprise genomic DNA sequences corresponding to a plurality of genes known to be imprinted in the subject; (b) providing a second data set comprising a plurality of nucleic acid sequences, wherein the nucleic acid sequences comprise genomic DNA sequences corresponding to a plurality of genes known not to be imprinted in the subject; (c) identifying one or more features that by themselves or in combination are differentially present or absent from the first data set as compared to the second data set; and (d) applying the one or more features to a test data set comprising a plurality of genomic DNA sequences which correspond to one or more genes for which the imprinting status is unknown to thereby identify an imprinted gene in a subject. The presently disclosed subject matter also provides methods for identifying a feature in a subject with respect to an imprinted gene and methods for detecting a presence of or a susceptibility to a medical condition associated with parent-of-origin dependent monoallelic expression in a subject.

### CROSS REFERENCE TO RELATED APPLICATION

The presently disclosed subject matter claims the benefit of U.S. Provisional Patent Application Ser. No. 60/873,151, filed Dec. 6, 2006; the disclosure of which is incorporated herein by reference in its entirety.

### GOVERNMENT INTEREST

This presently disclosed subject matter was made with U.S. Government support under Grant Nos. R01-ES008823 and R01-ES015165 awarded by the National Institutes of Health and Grant No. DE-FG02-05ER64101 from the Department of Energy. Thus, the U.S. Government has certain rights in the presently disclosed subject matter.

### TECHNICAL FIELD

The presently disclosed subject matter relates to the field of imprinted genes. More particularly, the presently disclosed subject matter relates to methods and compositions for identifying imprinted genes, for genotyping subjects with respect to one or more imprinted genes, for diagnosing and/or determining a susceptibility of a subject to a disease process associated with expression or lack of expression of an imprinted gene, and for determining those subjects predicted to benefit from therapies that target the epigenome.

### BACKGROUND

The untranslated mRNA H19 was the first gene shown to be imprinted in humans (Zhang & Tycko, 1992), and since its discovery in 1992, about 40 additional imprinted genes have been identified in the human genome (Morison et al., 2005). A gene is imprinted if the expression of one of its alleles is silenced or significantly reduced in expression depending on the parent from whom that allele was inherited (Reik & Walter, 2001). This functionally haploid state eliminates the protection that diploidy normally confers against the deleterious effects of recessive mutations. The expression of imprinted genes can also be deregulated epigenetically. Identifying genes that are imprinted in the human genome, and determining the factors responsible for epigenetic establishment and maintenance of imprinting control, remain as goals in the art.

Experimental identification of imprinted genes has typically focused on small genomic regions. These efforts are usually motivated by phenotypical observations, such as differences when a gene knock-out was inherited maternally versus paternally. The advent of cDNA microarrays to study differential expression between parthenogenetic and androgenetic embryos has allowed for a more high throughput approach (Mizuno et al., 2002; Nikaido et al., 2003). Though this general technique has led to the discovery of three apparently imprinted genes (Mizuno et al., 2002), it has recently been criticized for failing to enrich for truly imprinted genes because of the inherent expression differences associated with the abnormal development of parthenogenotes (Morison et al., 2005; Ruf et al., 2006).

Computational analyses have demonstrated that the concentration of certain types of repeated elements and other sequence characteristics can differ between monoallelically and biallelically expressed genes (Greally, 2002; Ke et al., 2002; Allen et al., 2003), yet there are no unique sequence motifs known to be common to imprinted genes. A machine learning approach was recently used to predict novel imprinted genes across the entire mouse genome using a variety of sequence-derived statistics (Luedi et al., 2005).

However, comparative models between mouse and human are complicated by discrepancies in imprinting status. For example, while some genes are imprinted in both mouse and human, others, including Igf2r, Ascl2, Phemx, Cd81, Tssc4, Nap1l4, Gatm, Dcn, and Impact are imprinted in mouse but not human (Morison et al., 2005; Monk et al., 2006). Conversely, the homeobox gene DLX5 is imprinted in human (Okita et al., 2003) but not mouse (Kimura et al., 2004), although a subtle maternal preference was reported in the mouse brain (Horike et al., 2005). This discordance makes the mouse an unreliable model for identifying imprinted genes in humans.

Therefore, there exists a long-felt need in the art for methods and compositions for identifying imprinted genes in humans and for correlating of the same with disease processes.

To address this need at least in part, the presently disclosed subject matter provides methods and compositions for identifying imprinted genes. The genes so identified are useful for genotyping subjects to identify and/or detect disease processes that are associated with expression or lack of expression of an imprinted gene and/or for identifying a susceptibility of a subject to a disease process associated with expression or lack of expression of an imprinted gene, and for determining those subjects predicted to benefit from therapies that target the epigenome.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for identifying an imprinted gene in a subject. In some embodiments, the methods comprise (a) providing a first data set comprising a plurality of nucleic acid sequences, wherein the nucleic acid sequences comprise genomic DNA sequences corresponding to a plurality of genes known to be imprinted in the subject; (b) providing a second data set comprising a plurality of nucleic acid sequences, wherein the nucleic acid sequences comprise genomic DNA sequences corresponding to a plurality of genes known not to be imprinted in the subject; (c) identifying one or more features that by themselves or in combination are differentially present or absent from the first data set as compared to the second data set; and (d) applying the one or more features to a test data set comprising a plurality of genomic DNA sequences which correspond to one or more genes for which the imprinting status is unknown to thereby identify an imprinted gene in a subject. The genomic DNA sequences can include untranslated sequences of in some embodiments at least 1 kilobase, in some embodiments at least 2 kilobases, in some embodiments at least 5 kilobases, in some embodiments at least 10 kilobases, in some embodiments at least 25 kilobases, in some embodiments at least 50 kilobases, in some embodiments at least 100 kilobases, and in some embodiments greater than 100 kilobases for one or more of the plurality of genes known to be imprinted in the subject, one or more of the plurality of genes known not to be imprinted in the subject, and combinations thereof. In some embodiments, the genomic DNA sequences comprise 5′ untranslated sequences, 3′ untranslated sequences, or both 5′ and 3′ untranslated sequences. In some embodiments, the features are selected from those set forth in Table 4 hereinbelow. In some embodiments, the identifying comprises training an algorithm using the first data set as a first training data set and the second data set as a second training data set to thereby identify one or more features in the first and second data sets that are predictive of imprinting status.

The presently disclosed subject matter also provides methods for identifying a feature in a subject with respect to an imprinted gene. In some embodiments, the methods comprise (a) obtaining a biological sample from the subject, wherein the biological sample comprises one or more nucleic acid molecules derived from one or more of the genes present within the genome of the subject (including, but not limited to those genes listed in Tables 1 and/or 7 hereinbelow); and (b) analyzing the one or more nucleic acid molecules, whereby a feature is identified in the subject with respect to the imprinted gene. In some embodiments, the feature is selected from the group consisting of a genetic feature, an epigenomic feature, and combinations thereof. In some embodiments, the genetic feature comprises a genotype of the subject with respect to at least one gene (e.g., one of the genes listed in Tables 1 and/or 7 hereinbelow). In some embodiments, the epigenomic feature is selected from the group consisting of a DNA sequence modification (e.g., methylation), a nucleosome positioning feature, a chromatin state, and a histone modification (e.g., methlyation, acetylation, etc.). In some embodiments, the biological sample comprises genomic DNA from the subject. In some embodiments, the analyzing comprises sequencing at least a portion of the one or more nucleic acid molecules derived from one or more of the genes present within the genome of the subject (e.g., one or more of the genes listed in Tables 1 and/or 7 hereinbelow). In some embodiments, the subject is heterozygous for one or more polymorphisms located in the portion of the one or more nucleic acid molecules derived from one or more of the genes present within the genome of the subject (including, but not limited to the genes listed in Tables 1 and/or 7 hereinbelow), and the sequencing identifies the one or more polymorphisms.

In some embodiments, the methods further comprise screening a biological sample from one or both biological parents of the subject to identify which parent transmitted each allele to the subject. In some embodiments, the methods further comprise predicting whether or not one or more of the alleles is likely to be expressed in the subject. In some embodiments, the predicting comprises correlating maternal or paternal inheritance of the one or more alleles with an assessment of whether the one or more alleles is expressed when inherited maternally or paternally.

The presently disclosed subject matter also provides methods for detecting a presence of or a susceptibility to a medical condition associated with parent-of-origin dependent monoallelic expression in a subject. In some embodiments, the methods comprise (a) obtaining a biological sample from the subject, wherein the biological sample comprises one or more nucleic acid molecules; (b) analyzing the one or more nucleic acid molecules for a feature with respect to parent-of-origin for one or both alleles of at least one imprinted gene; and (c) determining whether the feature correlates with a presence of or a susceptibility to a medical condition associated with monoallelic expression, whereby a presence of or a susceptibility to a medical condition associated with parent-of-origin dependent monoallelic expression in the subject is detected. In some embodiments, the feature is selected from the group consisting of a genetic feature, an epigenomic feature, and combinations thereof. In some embodiments, the genetic feature comprises a genotype of the subject with respect to at least one gene (e.g., a gene listed in Tables 1 and/or 7 hereinbelow). In some embodiments, the epigenomic feature is selected from the group consisting of a DNA sequence methylation state, a nucleosome positioning feature, and a histone modification. In some embodiments, the feature relates to a gene (e.g., a gene listed in Tables 1 and/or 7) the expression or lack of expression of which is associated with a medical condition. In some embodiments, the medical condition is selected from the group consisting of alcoholism, Alzheimer's disease, asthma/atopy, autism, bipolar disorder, obesity, diabetes, Parental Uniparental Disomy (UPD), cancer, epilepsy, DiGeorge syndrome, and schizophrenia. In some embodiments, the at least one imprinted gene is selected from DLGAP2 and KCNK9.

In some embodiments of the presently disclosed methods, the subject is a mammal, and in some embodiments the subject is a human.

It is an object of the presently disclosed subject matter to provide a method for identifying imprinted genes.

An object of the presently disclosed subject matter having been stated hereinabove, and which is achieved in whole or in part by the presently disclosed subject matter, other objects will become evident as the description proceeds when taken in connection with the accompanying examples and drawings as best described hereinbelow.

### BRIEF DESCRIPTION OF THE DRAWINGS

FIGS. 1A-1C are schematic diagrams depicting the genome-wide distribution of genes proved (filled triangles) or predicted with high confidence (unfilled triangles) to be imprinted. Downward triangles, upward triangles, and circles indicate genes predicted to be maternally, paternally, or biallelically expressed, respectively. Gray bars highlight a 3 Mb region centered on the linkage regions presented in Table 7 hereinbelow.

FIGS. 2A-2E and 3A-3E present a series of bar graphs depicting distributions of the weights of features characteristic of imprinted genes, as determined by two feature selection methods, those of Equbits (FIGS. 2A-2E) and SMLR (FIGS. 3A-3E). Absolute weights are shown as box plots; the dotted line represents the overall mean of all selected features. FIGS. 2A and 3A are bar graphs depicting distributions of feature type. FIGS. 2B and 3B are bar graphs depicting distributions of different ways of quantifying repetitive elements. Ratios of ±counts carried the greatest weight (P<6×10⁻¹¹). FIGS. 2C and 3C are bar graphs depicting distributions of different repetitive element locations. The 1 kb downstream window was of least importance (P<1×10⁻³). FIGS. 2D and 3D are bar graphs depicting distributions of different families of repetitive elements. Alus carried the lowest weight (P<4×10⁻³), whereas endogenous retroviruses were of greatest importance (P<3×10⁻³). FIGS. 2E and 3E are bar graphs depicting distributions of counts of the highest scoring transcription factor binding sites.

FIGS. 4A and 4B are plots depicting sequence comparisons of conceptus and maternal genomic DNA versus conceptus cDNA. In each plot, the arrow denotes the polymorphic nucleotide position.

FIG. 4A depicts results showing a conceptus as polymorphic (G/A, GENBANK® Accession No. rs17829155, now merged with SNP ID rs2235112; SEQ ID NO: 1) in DLGAP2, whereas the mother (maternal decidua) is homozygous (A/A). Thus, DLGAP2 isoforms 24, 25, 26, and 27 are expressed monoallelically in the testis from the paternal allele.

FIG. 4B depicts results showing a conceptus as polymorphic (C/T, GENBANK® Accession No. rs2615374; SEQ ID NO: 2) in KCNK9, whereas the mother (maternal decidua) is homozygous (C/C) at the polymorphic nucleotide position. Thus, KCNK9 is expressed monoallelically in the brain from the maternal allele.

FIG. 5 is a flow chart illustrating schematically the processes of cross-validation, training, testing, and prediction under two different kernels and employing Equbits and SMLR classifier learning strategies.

### BRIEF DESCRIPTION OF THE SEQUENCE LISTING

SEQ ID NO: 1 is a nucleic acid sequence of GENBANK® Accession No. rs17829155 (now merged with SNP ID rs2235112, which lists the SNP from the opposite strand as set forth herein), a polymorphism associated with the DLGAP2 locus.

SEQ ID NO: 2 is a nucleic acid sequence of GENBANK® Accession No. rs2615374, a polymorphism associated with the KCNK9 locus.

SEQ ID NOs: 3-13 are the nucleotide sequences of various primers that can be employed in the analysis of the DLGAP2 and KCNK9 loci and gene products thereof.

### DETAILED DESCRIPTION

### I. General Considerations

Imprinted genes can be essential in embryonic development, and imprinting dysregulation can contribute to human disease (Murphy & Jirtle, 2003). Disclosed herein are 156 human genes predicted to be imprinted by multiple classification algorithms using DNA sequence characteristics as features. Two of these genes have been verified experimentally to indeed be imprinted in humans. KCNK9, which is predominantly expressed in the brain, might be involved in bipolar disorder and epilepsy (Kananura et al., 2002), and is a known oncogene (Patel & Lazdunski, 2004), while DLGAP2 is a candidate bladder cancer tumor suppressor (Muscheck et al., 2000). The findings disclosed herein demonstrate that DNA sequence characteristics, including recombination hot spots, are sufficient to accurately predict the imprinting status of individual genes in the human genome. Moreover, mapping the imprinted gene candidates onto the chromosomal landscape defined by linkage analysis revealed many to be in loci that are linked to human health conditions as diverse as alcoholism, Alzheimer's, asthma, autism, bipolar disorder, cancer, diabetes, obesity, and schizophrenia.

Genes involved in human disease are commonly identified by disease-oriented experimental approaches. Disclosed herein is the discovery that potential susceptibility genes for a wide range of conditions can be identified by defining the subset of genes that are functionally haploid because of imprinting. Mapping these imprinted genes to disease susceptibility loci with parent-of-origin inheritance provides novel insights into how complex human diseases can arise from environmental alteration of the epigenome.

Thus, in some embodiments the presently disclosed subject matter provides a model to perform genome-wide predictions of imprinted genes directly in the human. These predictions are then employed to guide experimental identifications of new imprinted human genes.

### II. Definitions

Unless otherwise defined, all technical and scientific terms used herein have the same meaning as commonly understood by one of ordinary skill in the art to which the presently disclosed subject matter pertains. For clarity of the present specification, certain definitions are presented hereinbelow.

Following long-standing patent law convention, the articles “a”, “an”, and “the” refer to “one or more” when used in this application, including in the claims. For example, the phrase “a polymorphism” refers to one or more polymorphisms. Similarly, the phrase “at least one”, when employed herein to refer to an oligonucleotide, a gene, or any other entity, refers to, for example, 1, 2, 3, 4, 5, 6, 7, 8, 9, 10, 15, 20, 25, 50, 100, or more of that entity. Thus, the phrase “at least one gene” used in the context of the genes and gene products disclosed herein refers to 1, 2, 3, 4, 5, 6, 7, 8, 9, 10, up to every gene disclosed herein, including every value in between.

As used herein, the phrase “biological sample” refers to a sample isolated from a subject (e.g., a biopsy) or from a cell or tissue from a subject (e.g., RNA and/or DNA isolated therefrom). Biological samples can be of any biological tissue or fluid or cells from any organism as well as cells cultured in vitro, such as cell lines and tissue culture cells: Frequently the sample will be a “clinical sample” which is a sample derived from a patient (i.e., a subject undergoing a diagnostic procedure and/or a treatment). Typical clinical samples include, but are not limited to, blood, blood cells (e.g., white cells), tissue or fine needle biopsy samples, and cells therefrom. Biological samples can also include sections of tissues, such as frozen sections or formalin fixed sections taken for histological purposes. In some embodiments, a biological sample isolated from a subject comprises a number of cells to provide a sufficient amount of genomic DNA and/or RNA to practice one or more of the presently disclosed methods.

As used herein, the term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction. Unless specifically indicated to the contrary, the term “complementary” as used herein refers to 100% complementarity throughout the length of at least one of the two antiparallel nucleotide sequences.

As used herein, the phrase “derived from” refers to an entity that is present either in another entity and/or in some embodiments in the same entity but in a different context. In terms of biological samples and nucleic acids, the phrase “derived from” can be synonymous with “isolated from”. However, especially in the case of a biological molecule, the phrase “derived from” can also refer to the fact that the biological molecule is present in a different context or form in one situation versus another. For example, in some embodiments, the presently disclosed methods employ nucleic acid molecules “derived from” a gene (e.g., a gene listed in any of the Tables disclosed herein). In this context, it is understood that a nucleic acid molecule is “derived from” a gene if the nucleic acid molecule can be generated naturally or artificially by employing genetic and/or epigenomic information that is associated with the gene in the subject. In some embodiments, a nucleic acid molecule is “derived from” a gene if it is encoded by the gene, is a transcription product of the gene, or otherwise is generated based on genetic or non-genetic information that is provided by the gene.

As used herein, the term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an imprint regulatory element, an enhancer, an origin of replication, a 5′ or 3′ untranslated region, a coding region, and/or a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc.

As used herein, the term “gene” is used broadly to refer to any segment of DNA associated with a biological function. Thus, genes include, but are not limited to, coding sequences, the regulatory sequences required for their expression (e.g., 5′ regulator sequences, 3′ regulatory sequences, and combinations thereof), intron sequences associated with the coding sequences, and combinations thereof. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The phrase “hybridizing specifically to” refers to the binding, duplexing, or hybridizing of a molecule only to a particular nucleotide sequence under stringent conditions when that sequence is present in a complex mixture (e.g., total cellular) of DNA and/or RNA. The phrase “bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, the term “isolated”, when used in the context of an isolated nucleic acid or an isolated polypeptide, is a nucleic acid or polypeptide that, by the hand of man, exists apart from its native environment and is therefore not a product of nature. An isolated nucleic acid molecule or polypeptide can exist in a purified form or can exist in a non-native environment such as, for example, in a transformed host cell.

As used herein, the term “native” refers to a gene that is naturally present in the genome of an untransformed cell. Similarly, when used in the context of a polypeptide, a “native polypeptide” is a polypeptide that is encoded by a native gene of an untransformed cell's genome. Thus, the terms “native” and “endogenous” are synonymous.

As used herein, the term “naturally occurring” refers to an object that is found in nature as distinct from being artificially produced or manipulated by man. For example, a polypeptide or nucleotide sequence that is present in an organism (including a virus) in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., 1991; Ohtsuka et al., 1985; Rossolini et al., 1994). The terms “nucleic acid” or “nucleic acid sequence” can also be used interchangeably with gene, cDNA, and mRNA encoded by a gene.

As used herein, the phrase “oligonucleotide” refers to a polymer of nucleotides of any length. In some embodiments, an oligonucleotide is a primer that is used in a polymerase chain reaction (PCR) and/or reverse transcription-polymerase chain reaction (RT-PCR), and the length of the oligonucleotide is typically between about 15 and 30 nucleotides. In some embodiments, the oligonucleotide is present on an array and is specific for a gene of interest. In whatever embodiment that an oligonucleotide is employed, one of ordinary skill in the art is capable of designing the oligonucleotide to be of sufficient length and sequence to be specific for the gene of interest (i.e., that would be expected to specifically bind only to a product of the gene of interest under a given hybridization condition).

As used herein, the phrase “percent identical”,” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection. See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the term “subject” refers to any organism for which analysis of gene expression would be desirable. Thus, the term “subject” is desirably a human subject, although it is to be understood that the principles of the presently disclosed subject matter indicate that the presently disclosed subject matter is effective with respect to invertebrate and to all vertebrate species, including Therian mammals (e.g., Marsupials and Eutherians), which are intended to be included in the term “subject”. Moreover, a mammal is understood to include any mammalian species in which detection of differential gene expression is desirable, particularly agricultural and domestic mammalian species. The methods of the presently disclosed subject matter are particularly useful in the analysis of gene expression in warm-blooded vertebrates, e.g., mammals.

More particularly, the presently disclosed subject matter can be used for assessing imprinting and its consequences in a mammal such as a human. Also provided is the analysis of gene expression in mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses (e.g., thoroughbreds and race horses).

Additionally, in some embodiments the term “subject” refers to a biological sample as defined herein, which includes but is not limited to a cell, tissue, or organ that is isolated from an organism. Thus, it is understood that the methods and compositions disclosed herein can be employed for assessing imprinting and its consequences in a subject that is an organism but can also be employed for assessing imprinting and its consequences in a subject that is a biological sample isolated from an organism. Accordingly, the methods and compositions disclosed herein are intended to be applicable to assessing imprinting and its consequences in vivo as well as in vitro.

### III. Methods for Identifying an Imprinted Gene

The presently disclosed subject matter provides in some embodiments methods for identifying an imprinted gene in a subject. In some embodiments, the methods comprise a computer-assisted comparison of various features of genetic loci that are known to be imprinted to various features of genetic loci that are known not to be imprinted, and extrapolating from the comparison a plurality of features that are indicative of imprinting status.

As used herein, the term “identifying an imprinted gene” refers to predicting whether or not the gene is imprinted and/or if it is, predicting whether the gene is likely to be maternally or paternally expressed. In some embodiments, the identifying is accomplished by feature selection and classifier learning as described herein. In some embodiments, once features are selected and classifiers are learned, the learned classifiers, which are equations that output a value indicating the probability of being imprinted, are applied to the features of the genes in the genome.

As used herein, the term “imprinted” and grammatical variants thereof refers to a genetic locus for which one of the parental alleles is repressed and the other one is transcribed and expressed, and the repression or expression of the allele depends on whether the genetic locus was maternally or paternally inherited. Thus, an imprinted genetic locus is characterized by parent-of-origin dependent monoallelic expression: the two alleles present in an individual are subject to a mechanism of transcriptional regulation that is dependent on which parent transmitted the allele. Imprinting has been shown to be species- and tissue-specific as well as a developmental-stage-specific phenomenon (see e.g., Weber et al., 2001; Murphy & Jirtle, 2003).

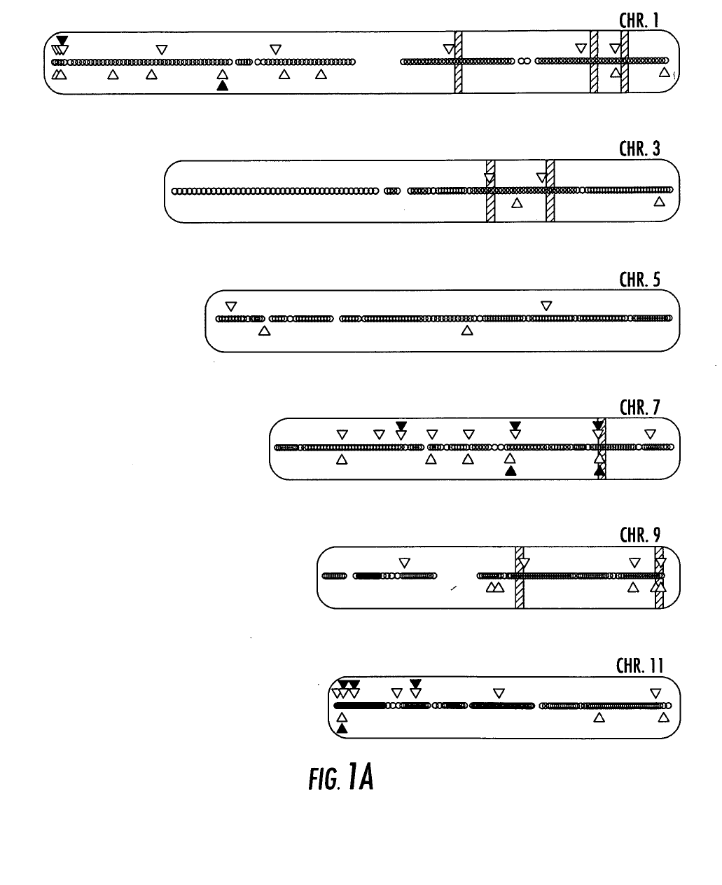

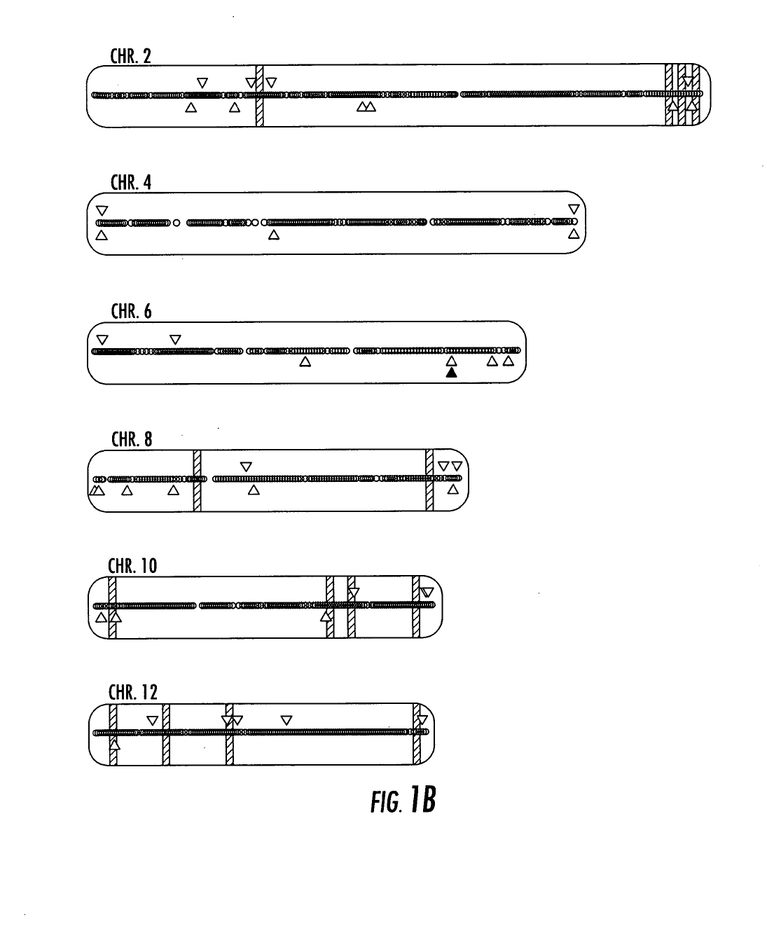

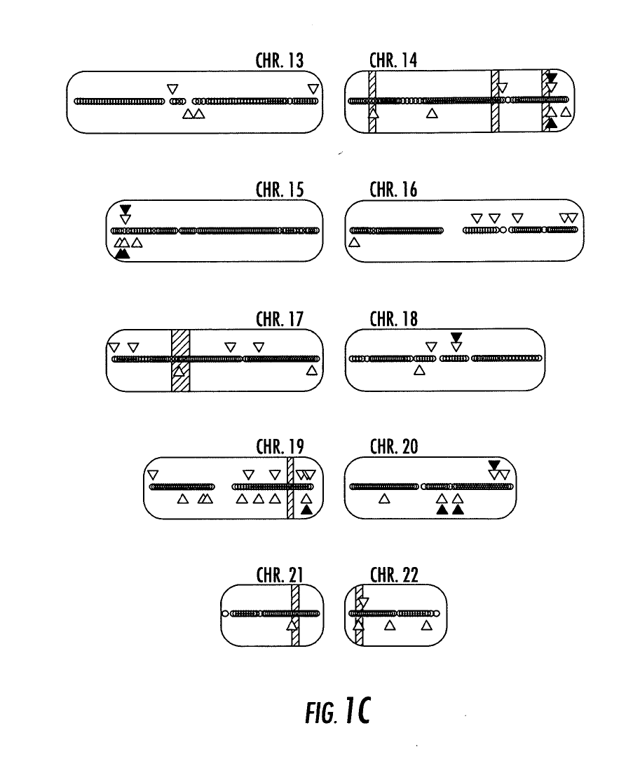

Several mechanisms by which genetic loci are imprinted have been identified, the most common of which appears to be differences in the methylation status of maternal and paternal alleles. However, and as disclosed herein, additional representative sequence features present within the genome have also been identified as being highly predictive of imprinting. These features are summarized in Table 4 hereinbelow. FIG. 1 depicts the distributions and weights of various features characteristic of imprinted genes as determined using two different algorithmic approaches. These features include, but are not limited to the presences and relative locations of various repetitive elements (e.g., Alu, CR1, FAM, FLAM, FRAM, HAL1, L1, L2, LTR, ERV, ERV1, ERVK, WRVI, MaLR, and MIR elements), their orientations relative to each other and to the direction of transcription, etc.

Thus, in some embodiments the presently disclosed methods comprise employing training algorithms to recognize the presence or absence of various genomic sequence features in known imprinted versus known non-imprinted genes, and to use the trained algorithms to identify whether a genetic locus that might or might not be imprinted is in fact imprinted or not. In some embodiments, the methods comprise (a) providing a first data set comprising a plurality of nucleic acid sequences, wherein the nucleic acid sequences comprise genomic DNA sequences corresponding to a plurality of genes known to be imprinted in the subject; (b) providing a second data set comprising a plurality of nucleic acid sequences, wherein the nucleic acid sequences comprise genomic DNA sequences corresponding to a plurality of genes known not to be imprinted in the subject; (c) identifying one or more features that by themselves or in combination are differentially present or absent from the first data set as compared to the second data set; and (d) applying the one or more features to a test data set comprising a plurality of genomic DNA sequences which correspond to one or more genes for which the imprinting status is unknown to thereby identify an imprinted gene in a subject. Representative human genes that are known to be imprinted or non-imprinted and that can be used to train the algorithms are presented in Tables 8 and 9.

IV. Methods for Identifying Genetic and Epigenomic Features in a Subject with Respect to an Imprinted Gene

The presently disclosed subject matter also provides methods for identifying a feature in a subject with respect to an imprinted gene. In some embodiments, the methods comprise (a) obtaining a biological sample from the subject, wherein the biological sample comprises one or more nucleic acid molecules isolated from the subject (e.g., a nucleic acid molecule derived from and/or encoding one or more of the genes listed in Tables 1 and/or 7 hereinbelow); and (b) analyzing the one or more nucleic acid molecules, whereby a feature is identified in the subject with respect to the imprinted gene

As used herein, the term “feature” refers to any assayable and/or identifiable characteristic of a genome or epigenome of the subject. Exemplary, non-limiting features include genetic features such as DNA sequence differences (e.g., genotypes).

As such, in some embodiments the presently disclosed methods relate to genotyping a subject with respect to an imprinted gene. As used herein, the phrase “genotyping a subject with respect to an imprinted gene” refers to determining what alleles the subject has with respect to an imprinted gene, and further whether the individual alleles were inherited maternally or paternally. After this has been determined, it can be possible to predict a phenotype that is associated with the genotype.

Any method can be used to determine a genotype with respect to an imprinted gene. In some embodiments, the methods rely on there being an assayable difference between the alleles. Exemplary assayable differences include sequence differences (for example, nucleotide sequence differences in the open reading frame of an imprinted gene, including but not limited to those that result in amino acid differences in the encoded polypeptide). The sequence differences can be determined directly (for example, by sequencing and/or by using amplification primers that are specific for different alleles) or can be determined indirectly (for example, by assaying a biological activity or a biochemical characteristic of a nucleic acid sequence and/or a polypeptide encoded thereby).

Once an assayable characteristic of each allele is determined, it is also possible to determine from which parent each allele is inherited. For example, a sequence difference identified in an imprinted gene in a subject can be used to assay one or both parents to determine what alleles the parents have, and by deduction which alleles in the subject came from which parents.

For example, with imprinted genes it is possible to disregard any contribution to a phenotype from an allele that is expected not to be expressed as a result of the imprinting. In some embodiments, including for example where the imprinting results in monoallelic expression only in a tissue-specific and/or developmental-stage-specific expression of an imprinted gene, this can result in a phenotype in the subject (for example, in a specific cell type or tissue or at a specific developmental stage) that can be predicted once a genotype including parent-of-origin is known.

This approach can also benefit from knowing whether the maternal or paternal allele is expected to be expressed in the cell or tissue type of interest or at the developmental stage of interest. A method for predicting parental preference is disclosed herein (see e.g., EXAMPLE 7).

Additionally, a feature that is identified can be an epigenomic feature. Representative, non-limiting epigenomic features include DNA sequence modifications other than nucleotide changes (e.g., methylation status), nucleosome positioning features, chromatin states, and histone modifications (e.g., methlyation or acetylation status or similar). Techniques for assaying for the presence of these epigenomic features would be known to one of ordinary skill in the art after consideration of the present disclosure.

V. Methods for Detecting the Presence of, or Predicting a Susceptibility to, a Medical Condition Associated with Parent-of-Origin Dependent Monoallelic Expression

The presently disclosed subject matter provides in some embodiments methods for detecting a presence of, or predicting a susceptibility to, a medical condition associated with parent-of-origin dependent monoallelic expression in a subject. In some embodiments, the methods comprise (a) obtaining a biological sample from the subject, wherein the biological sample comprises one or more nucleic acid molecules; (b) analyzing the one or more nucleic acid molecules for a feature with respect to parent-of-origin for one or both alleles of at least one imprinted gene; and (c) determining whether the feature correlates with a presence of or a susceptibility to a medical condition associated with monoallelic expression, whereby a presence of or a susceptibility to a medical condition associated with parent-of-origin dependent monoallelic expression in the subject is detected

Stated another way, the presently disclosed subject matter provides in some embodiments methods for correlating a subject's genotype with respect to one or more imprinted genes with a disease phenotype based on which alleles for the one or more imprinted genes are inherited maternally and which are inherited paternally.

It is possible for subjects to have and/or be susceptible to medical conditions that are associated with imprinted genes. For example, because imprinted genes are expressed in a parent-of-origin dependent monoallelic fashion (in some embodiments the monoallelic expression being tissue- and/or developmental stage-specific), it is possible for a subject to inherit a deleterious allele of an imprinted gene from one parent that is not compensated for by the allele inherited from the other parent. In these cases, it is useful to know not only the nature of the two alleles that a subject has, but also the parent from whom the subject has inherited each allele. Examples of medical conditions that might be associated with imprinted genes include, but are not limited to alcoholism, Alzheimer's disease, asthma/atopy, autism, bipolar disorder, obesity, diabetes, Parental Uniparental Disomy (UPD), cancer, epilepsy, DiGeorge syndrome, and schizophrenia (see e.g., Table 7 hereinbelow). In some embodiments, the imprinted gene is DLGAP2, DLGAP2L, KCNK9, RTL1.

In some embodiments, the presently disclosed methods can be employed for determining those subjects predicted to benefit from therapies that target the epigenome. As used herein, the term “epigenome” refers to the overall epigenetic state of a subject and/or of a particular, cell, tissue, or organ thereof.

Thus, in some embodiments the epigenome relates to the sum total of all genetic effects as well as epigenetic effects, the latter of which result in some embodiments from differences in expression of loci that are subject to parent-of-origin dependent monoallelic expression. In some embodiments, a subject that is predicted to be likely to benefit from therapies that target the epigenome is a subject in which a cell, tissue, or organ functions inappropriately as a result of the dysregulation of parent-of-origin dependent monoallelic expression of one or more loci. In some embodiments, the one or more genetic loci are selected from among those loci set forth in Table 1 or Table 2 hereinbelow. In some embodiments, the inappropriate function in the cell, tissue, or organ results in the subject having one or more of the conditions set forth in Table 7 hereinbelow. In some embodiments, the condition comprises cancer (see Yoo & Jones, 2006; Feinberg et al., 2006).

Additionally, the phrase “therapies that target the epigenome” refers to therapies that are designed to influence at least one effect of the epigenome on a phenotype in a subject (e.g., a phenotype related to a disorder or other undesirable medical condition). In some embodiments, a therapy that targets the epigenome can comprise administering to a subject in need thereof a composition that can modify the methylation and/or acetylation of an imprint regulatory element of an imprinted locus. Exemplary, non-limiting examples of such compositions include methyl donors, modulators of methyl transferases, acetyl donors, and modulators of acetylases.

### Examples

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Human Genome Data

DNA sequence and annotation data were obtained from the Ensembl database, jointly managed by the European Molecular Biology Laboratory—European Bioinformatics Institute (EMBL—EBI; Cambridge, United Kingdom) and the Sanger Institute (Cambridge, United Kingdom). It is publicly available on the World Wide Web. A positive training set of 40 imprinted genes compiled from the Imprinted Gene Catalog (publicly available from the website of the University of Otago, Dunedin, New Zealand) and recent literature, and a negative training set of 52 genes, for which experimental evidence suggests biallelic expression was employed. Additionally, random sets of 500 control genes presumed to be non-imprinted for a number of tasks were also employed. These random control genes were sampled from autosomal chromosomal bands known or not suspected to contain imprinted genes, and were intended to represent the overall characteristics of biallelically expressed genes. Random control genes were used to compute top pairwise interaction terms, to carry out feature selection with the Equbits classifier (Equbits Inc., Livermore, Calif., United States of America), and to supplement the final training set that was used to learn our classifiers. To minimize bias, the set of 500 random control genes was resampled for each of these three tasks.

### Example 2

### Feature Measurements

DNA sequence feature measurements were acquired from an examination of human genomic sequences present in the Ensembl database and included data derived from recombination hotspots, nucleosome formation potential, and repeat phase changes, as explained below.

Another statistic regarding the repetitive elements flanking a gene was introduced, which is referred to as “phase change” and is defined as an instance of a repetitive element changing its orientation compared to a neighboring element of the same family. The number of such phase changes was counted among retrotransposon classes such as Alus, MIRs, and LTRs within the 100 kb up- and downstream. In doing this, it was noticed that within the downstream region of imprinted genes, compared to a random sample, a phase change occurred more frequently in one of the following LTRs: MLT1A0, MLT1B, MSTA, MSTB1, MLT1D, MLT2B4, or MLT1G1. Conversely, phase changes in an MLT1C LTR were underrepresented in the flanking regions of imprinted genes.

Whether data on recombination could be used to discern imprinted genes was also investigated. Coordinates of recombination hotspots (Myers et al., 2005) were downloaded from the International HapMap Project website. The recombination hotspots were mapped to the data set, and for each gene the number of hotspots within 350 kb up- and downstream, as well as the minimum distance to the closest recombination hotspot up- and downstream were computed. Interestingly, the retrovirus-like retrotransposon THE1B is reported to be among certain sequence features that are overrepresented in hotspots (Myers et al., 2005). In particular, Myers et al. found the 8-nucleotide motif CCACGTGG to be significantly more frequent in hotspot THE1Bs compared to THE1Bs elsewhere in the genome. The same oligonucleotide motif is also involved in serum-induced transcription at the G1/S-phase boundary in the hamster (Miltenberger et al., 1995), and is known as the G-box binding motif for plant basic leucine zipper (bZIP) proteins (Niu et al., 1999). The occurrence of this oligomer within all THE1B elements in the 100 kb flanking each gene was counted.

The last additional class of feature measurements involved nucleosome formation potential profiles. Such in silico estimates of nucleosome packaging density in the promoter region have previously been used to distinguish tissue-specific genes from housekeeping genes and widely expressed genes (Levitsky et al., 2001). Nucleosome formation potential estimates were acquired and summarized as follows. The sum within the 0.82-0.61 kb upstream, the standard deviation 5.86-5.81 kb upstream, the mean 0-1 and 0.31-0.49 kb within the concatenated exons, and the standard deviation 6.7-6.75 and 7.02-7.07 kb downstream were computed. These particular windows were picked following visual inspection of plotted potentials.

### Example 3

### Statistical Methods

To be more robust in the imprinted gene predictions, two distinct strategies for feature selection and classifier learning were employed: Equbits FORESIGHT™ (Equbits Inc., Livermore, Calif., United States of America), which employs support vector machines, and Sparse Multinomial Logistic Regression (SMLR; Krishnapuram et al., 2005), which adopts a Bayesian approach to sparse multinomial logistic regression. In each case, two separate classifiers were learned: one with a linear kernel and one with a radial basis function (RBF) kernel. The operating point on the ROC for each classifier was chosen so as to minimize the number of false positives while retaining all true positives. To be more conservative in the final predictions, joint agreement among all four classifiers was required before predicting a gene to be imprinted. These are referred to herein as the “high-confidence” predictions.

When using Equbits to predict imprinted genes, a 40-fold cross-validation (CV) procedure was used; at each step feature selection was performed using a linear kernel and then classifiers for imprint status with linear and RBF kernels were learned. Based on the results of this CV, final parameters were selected and linear and RBF classifiers trained on the full training set were applied both to the independent test set and to the whole human genome. During CV, the number of retained features ranged from 613 to 638, while 626 features were retained in the final classifier.

When using SMLR to predict imprinted genes, a similar scheme was adopted. At each step of a 40-fold CV, feature selection was performed by applying a sparsity-promoting prior directly on the weights of the features (no kernel) and then classifiers for imprint status with linear and RBF kernels were learned. Based on the results of this CV, final parameters were selected and linear and RBF classifiers trained on the full training set were applied both to the independent test set and to the whole human genome. During CV, the number of retained features averaged 875, while 820 features were retained in the final classifier.

SMLR is written in portable Java, with a GUI, and is available with complete source code under a non-commercial use license from Duke University (Durham, N.C., United States of America). In addition, all data, and all scripts used to produce the SMLR results, are also available.

To ensure that no straightforward relationships within the training data were obscured by sophisticated learning methods, CV was also performed using three simple classifiers (as implemented in Weka 3.4; Witten & Frank, 2005). A naïve Bayes classifier showed a sensitivity of 40% (16 out of 40 imprinted genes correctly recognized) and a specificity of 97% (535 out of 552 non-imprinted genes correctly classified). A decision stump simply classified all genes as non-imprinted. A random forest classifier showed a sensitivity of 20% (eight out of 40 correct) and a specificity of 95% (522 out of 552 correct). These experiments suggested that simple alternative classification approaches were not likely to result in comparable classification accuracy.

To simplify the prediction of parental expression preference, Equbits was employed only with a linear kernel and the top 30 features. This procedure is analogous to that used to predict parental preference in the mouse (Luedi et al., 2005).

X²-tests were used to compare proportions and two-sided Student's t-tests to compare means. To be conservative, Bonferroni's method was used when correcting for multiple testing (α=0.05).

### Example 4

### Experimental Procedures

From human conceptuses and matched maternal deciduas, DNA was isolated in Qiagen buffer ATL and proteinase K (Qiagen Inc., Valencia, Calif., United States of America) followed by phenol-chloroform-isoamyl alcohol extraction and ethanol precipitation. Each individual was screened for polymorphisms in KCNK9 (C/T, dbSNP Accession No. rs2615374; SEQ ID NO: 2) and DLGAP2 (G/A, dbSNP Accession No. rs17829155 (now merged with SNP ID rs2235112); SEQ ID NO: 1) by genomic DNA PCR with Qiagen HOTSTARTAQ® polymerase (Qiagen Inc., Valencia, Calif., United States of America) as per the manufacturer's instructions. Following identification of heterozygous polymorphic individuals, total RNA was isolated from brain and testis by homogenization in RNA-Stat 60 (Tel-Test, Friendswood, Tex., United States of America); subsequent processing was performed as recommended by the manufacturer.

First strand cDNA was primed with gene-specific primers (see below), and synthesized from DNase I-treated RNA using SUPERSCRIPT® II as recommended by the manufacturer (Invitrogen, Carlsbad, Calif., United States of America). Qiagen HOTSTARTAQ® polymerase (Qiagen Inc., Valencia, Calif., United States of America) in a 25 μl RT-PCR reaction volume, as per the manufacturer's instructions. RT-PCR products were separated by electrophoresis on a 1.5% agarose gel, and appropriately sized fragments of cDNA were excised and gel-extracted (GENELUTE™, Sigma Chemical Co., St. Louis, Mo., United States of America). Products were sequenced (ABI 377 sequencer, PE Biosystems, Foster City, Calif., United States of America), and analyzed for expression using FinchTV (Geospiza, Inc., Seattle, Wash., United States of America).

In order to rule out any stochastic effects, the PCR and the sequencing reactions were repeated at least three times in all cases where exclusive monoallelic expression was observed. All sequencing reactions were also performed in both directions.

DLGAP2 (Disks large-associated protein 2), also known as DAP-2, is annotated to have four splice variants (see the University of California at Santa Cruz Genome Website, May 2004 assembly, Santa Cruz, Calif., United States of America; Karolchik et al., 2003). The four splice variants—chr8.27.24, chr8.27.25, chr8.27.26, and chr8.27.27—are referred to as DLGAP2-24, -25, -26, and -27, respectively. Isoforms DLGAP2-24 and DLGAP2-25 were reverse transcribed using primer DLGAP2-RT1 (SEQ ID NO: 3), while DLGAP2-RT2 (SEQ ID NO: 4) was used to reverse transcribe DLGAP2-26 and DLGAP2-27. cDNA from DLGAP2-24 and DLGAP2-27 was specifically amplified using reverse primer DLGAP2-M1R (SEQ ID NO: 5), while DLGAP2-M2R (SEQ ID NO: 6) was used to amplify DLGAP2-25 and DLGAP2-26. DLGAP2-M1F (SEQ ID NO: 7) was used as a common forward primer to amplify cDNA. When amplifying cDNA, the primers bridged two long introns, ruling out any potential influence of undigested genomic DNA. Genomic DNA was amplified and sequenced using DLGAP2-1F (SEQ ID NO: 8) and DLGAP2-1R (SEQ ID NO: 9).

KCNK9 (potassium channel, subfamily K, member 9), also known as TASK-3, is annotated to have one isoform. Primers KCNK9-1F (SEQ ID NO: 10) and KCNK9-1R (SEQ ID NO: 11) were used for the amplification of genomic DNA. cDNA was amplified using KCNK9-M1F (SEQ ID NO: 12) and -M1R (SEQ ID NO: 13), which bridge an 84 kb intron. Primer sequences are given in Table 11 hereinbelow. In order to rule out any stochastic effects, the PCR and the sequencing reactions were repeated multiple times whenever monoallelic expression was observed. All sequencing reactions were performed in both directions.

### Example 5

### Conceptual Approach

A conservative approach was adopted in identifying human imprinted genes because of their important role in disease etiology. Specifically, two separate classifier learning strategies—one based on support vector machines and the other sparse logistic regression—each with a different feature selection process, were adopted. With each strategy, classifiers with two different similarity kernels were classified: linear and radial basis function (RBF). Only genes predicted to be imprinted by all four classifiers were considered “high-confidence” predictions. Although all four classifiers use the same initial training set of known imprinted genes, the combined classifier approach helps to control for biases that might arise from different choices for feature selection, classifier learning, or similarity kernel.

All four classifiers were trained on DNA sequence features collected from 40 genes known to be imprinted in human and 52 genes known not to be imprinted in human (see Table 9 hereinbelow), plus 500 randomly selected genes suspected not to be imprinted in human (see Table 10 hereinbelow). The prediction accuracy of the combined classifier both by cross-validation and with an independent negative test set was assessed (see Table 8 hereinbelow). In a 40-fold cross-validation, a specificity of 100% (40/40 imprinted genes correctly identified) and a sensitivity of 99% (545/552 presumably non-imprinted genes correctly identified) was obtained. The independent negative test set consisted of 13 genes with random monoallelic expression and 88 genes with biallelic expression or synchronous replication, including four genes imprinted in mouse but not human. All 101 genes were correctly predicted to not be imprinted (see Table 8 hereinbelow; see also FIG. 5 for a schematic depiction of the workflow).

### Example 6

### Genome-Wide Prediction of Candidate Imprinted Genes

Applying the combined classifier to the entire human genome, 156 of 20,770 (0.75%) annotated autosomal genes not previously known to be imprinted (Ensembl v20) were predicted to be imprinted with high confidence (see Table 1 and Table 2 hereinbelow). Only chromosomes 7 and 11 showed a higher density of predicted and known imprinted genes compared to the rest of the autosome (P=0.0014 and P=0.0026, respectively, X² test with 1 df; see also FIG. 1).

Seven chromosomal bands contained a significantly higher density of imprinted gene candidates, including novel candidates related to various cancers (P<2×10⁻⁸, X² test with 1 df; see Table 3 hereinbelow). The clusters on 15q12 and 7q21.3 include known imprinted genes. Included in the 11p15.5 region were well know imprinted genes such as H19 and IGF2, and five novel candidates, located further distal, including PKP3, an oncogene involved in lung cancer (Furukawa et al., 2005). The cluster on 1p36.32 included the known imprinted gene TP73 along with the novel candidate PRDM16, which is associated with leukemia (Du et al., 2005). The ortholog of this gene was also predicted to be imprinted in mouse (Luedi et al., 2005). Chromosomal band 14q32.31 contained the known imprinted gene MEG3 along with the novel candidate RTL1, which is imprinted in the mouse (Seitz et al., 2003) and sheep (Charlier et al., 2001). The cluster of candidate genes on 10q26.3 included the novel candidate NKX6-2, which is preferentially expressed in the brain (Lee et al., 2001), and was predicted to be imprinted in the mouse (Luedi et al., 2005). NKX6-2, along with four neighboring candidate genes, was predicted to be maternally expressed. This region on 10q26 is 4.7-5.7 Mb from the marker D10S217, which is maternally linked to male sexual orientation (Mustanski et al., 2005). A germline differentially methylated region was found within this interval (coordinate 135.1 Mb; see Strichman-Almashanu et al., 2002), lending further support to the prediction of imprinted genes within the immediate vicinity of this region.

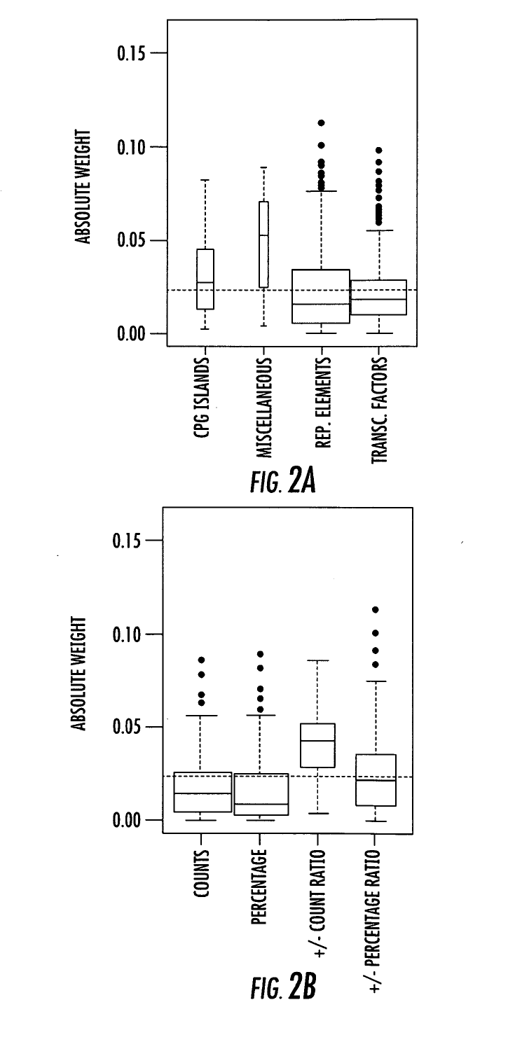

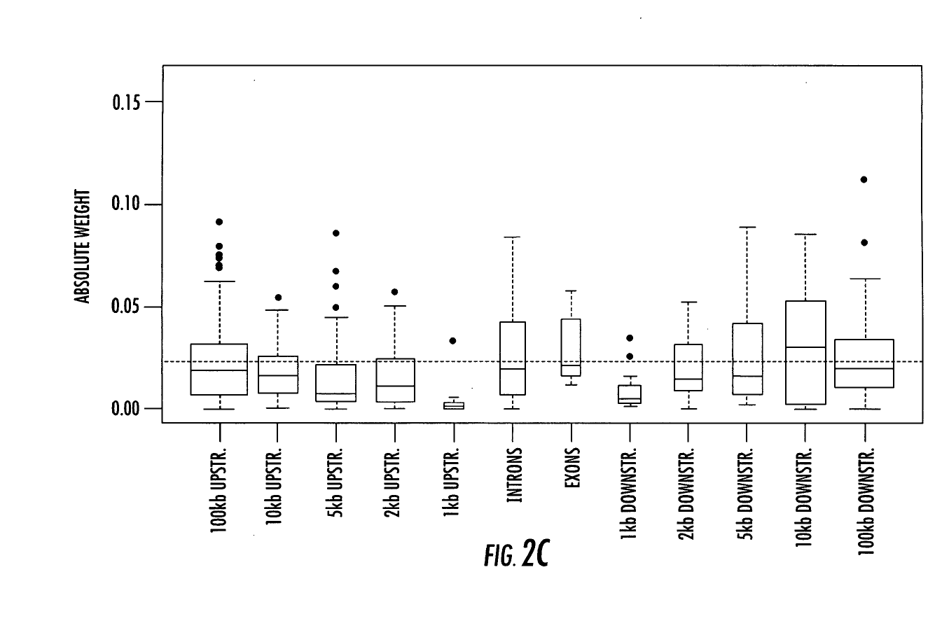

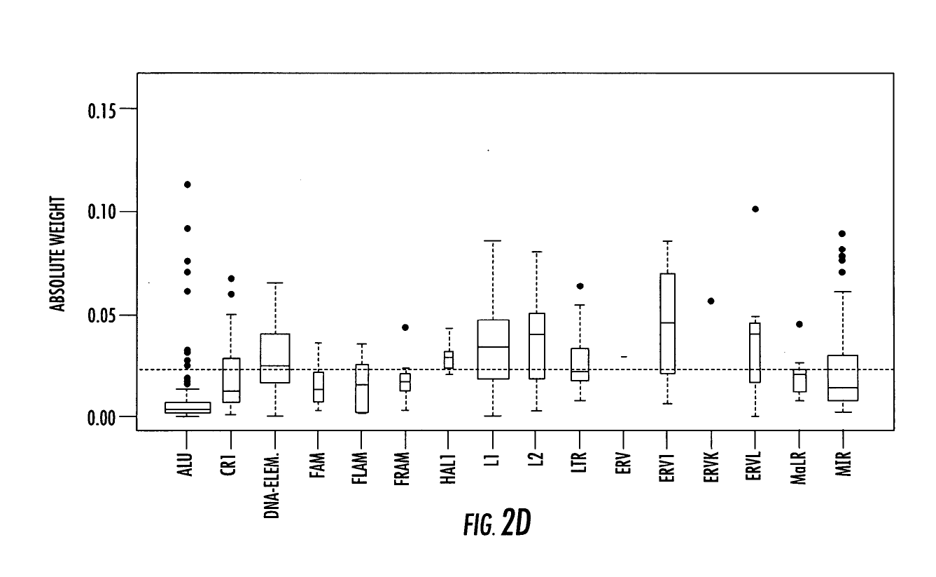

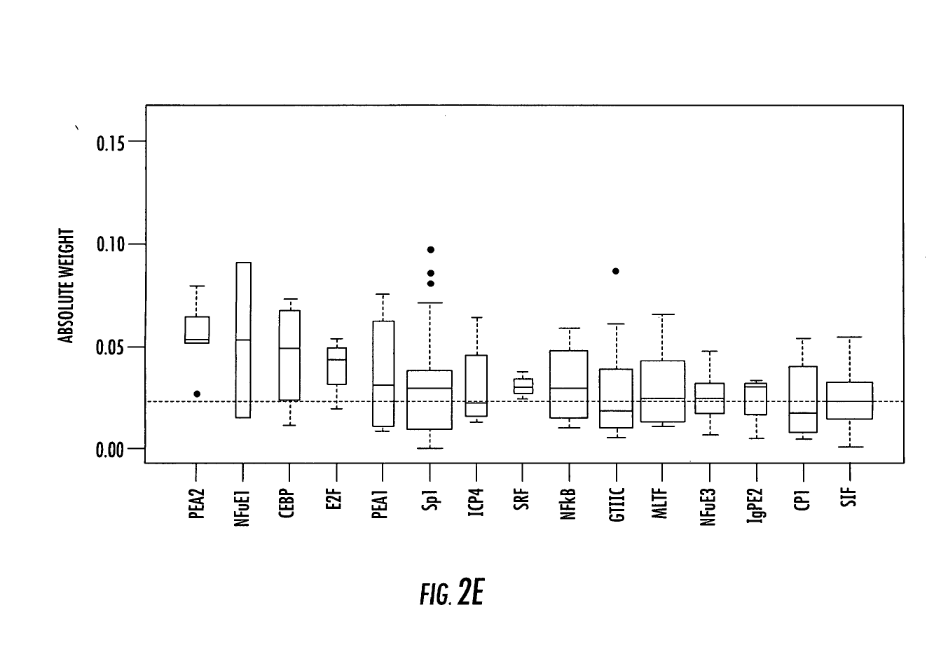

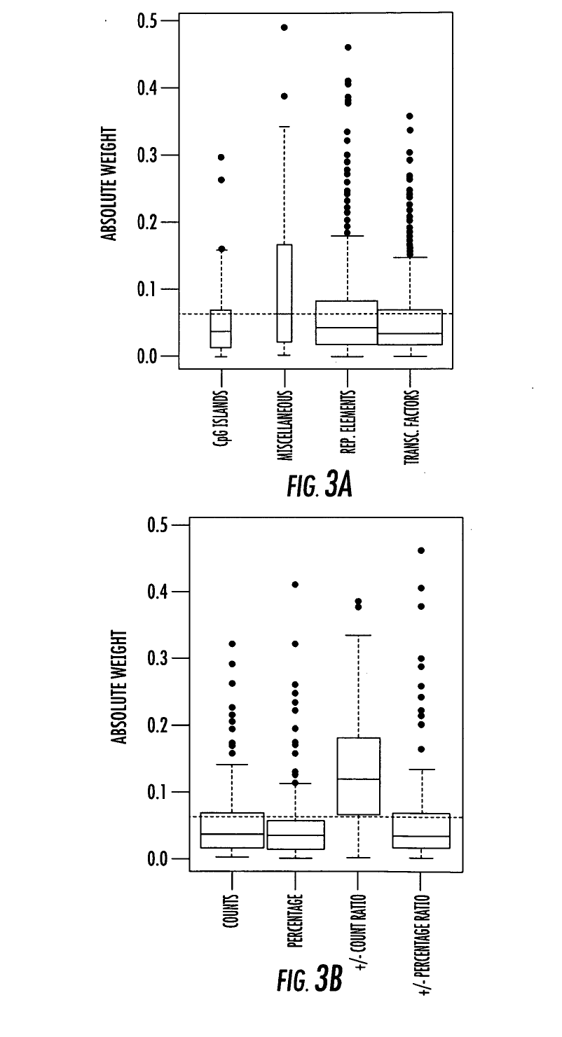

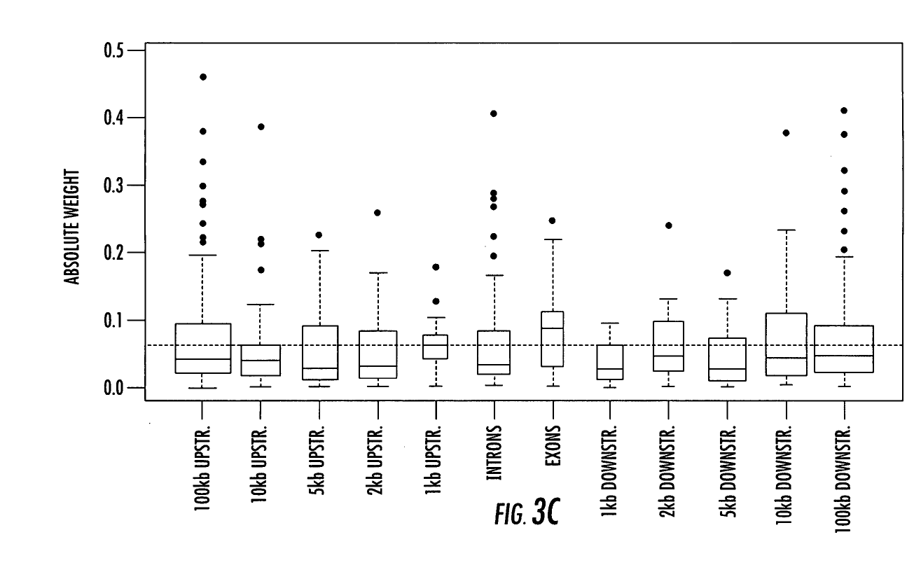

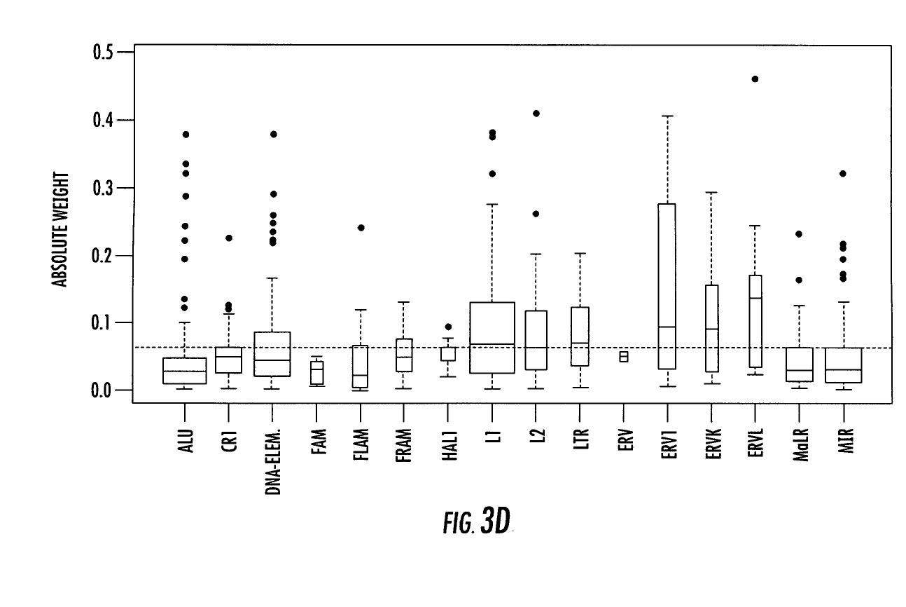

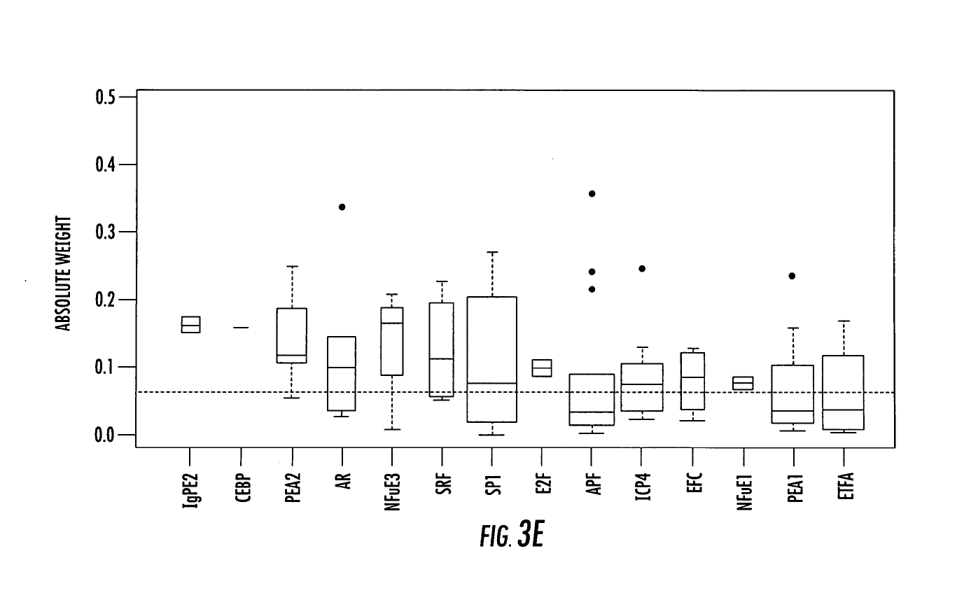

FIGS. 2 and 3 present a series of bar graphs depicting distributions of the weights of features characteristic of imprinted genes as determined by two feature selection methods: those of Equbits (FIG. 2) and SMLR (FIG. 3). Absolute weights are shown as box plots, the dotted line represents the overall mean of all selected features. FIGS. 2A and 3A depict the distribution of feature type. FIGS. 2B and 3B depict the distribution of different ways of quantifying repetitive elements. The ratios of ±counts carried the greatest weight (P<6×10⁻¹¹; see also Table 4 hereinbelow). FIGS. 2C and 3C depict the distribution of different repetitive element locations. The 1 kb downstream window was of least importance (P<1×10⁻³). FIGS. 2D and 3D depict the distribution of different families of repetitive elements. Alus carried the lowest weight (P<4×10⁻³), whereas endogenous retroviruses (ERV) were of greatest importance (P<3×10⁻³). FIGS. 2E and 3E depict the distribution of counts of the highest scoring transcription factor binding sites.

Among transcription factor binding sites, those of greatest importance in both feature selection strategies were CEBP, E2F, ICP4, IgPE2, NFuE1, NFuE3, PEA1, PEA2, Sp1, and SRF (see FIGS. 2E and 3E). E2F family transcription factors are involved with cell proliferation, Sp1 elements have been shown to protect CpG islands from de novo methylation in the embryo (Brandeis et al., 1994), and SRF (serum response factor) is involved in the activation of “immediate early” genes (Schratt et al., 2001), in muscle differentiation (Vandromme et al., 1992; Soulez et al., 1996), and in mesoderm formation (Arsenian et al., 1998).

### Example 7

### Prediction of Parental Preference

A separate classifier was trained to determine if the maternal or paternal allele of an imprinted gene is expressed. The training set included 19 maternally expressed genes and 20 paternally expressed genes (GRB10 was omitted due to its complex expression patterns (Blagitko et al., 2000)). In a 19-fold cross-validation, a sensitivity of 85% (17/20 paternally expressed genes correctly identified) and a specificity of 79% (15/19 maternally expressed genes correctly identified) was achieved. The ability to accurately predict the expressed parental allele of known imprinted genes in both human and mouse (Luedi et al., 2005) lent support to the suggestion that different mechanisms might be responsible for regulating paternal versus maternal imprinting (Mancini-Dinardo et al., 2006).

Maternal expression was predicted for 56% (88/156) of the candidate imprinted genes, comparable to the 64% frequency found for mouse imprinted genes (Luedi et al., 2005). Among the features of greatest significance for the prediction of parental expression preference were the ratios of the relative orientation of AluJ and ERVL elements downstream (see Table 5 hereinbelow). E4F1 transcription factor binding sites were also significantly more prevalent in the 3-4 kb upstream region of maternally expressed genes than in paternally expressed genes.

### Example 8

### Experimental Identification of New Imprinted Genes

Guided by the high-confidence predictions of the combined classifier, two new imprinted human genes were experimentally verified. DLGAP2 (Disks Large-Associated Protein 2) and KCNK9 (Potassium Channel, Subfamily K, Member 9) were chosen for experimental validation. A number of criteria were employed to prioritize the 156 predictions for experimental validation: large posterior probabilities of being imprinted (in the case of SMLR), large signed hyperplane distances (in the case of SVM), potential involvement in an important condition (such as a cancer or one of the conditions listed in Table 7), and location in a chromosome not known to contain imprinted genes (e.g., DLGAP2 and KCNK9 reside at opposite telomeric regions of chromosome 8, a human chromosome not previously shown to contain imprinted genes; Morison et al., 2005), as many imprinted genes have to date been identified by searching near known imprinted genes, so finding some on a completely different chromosome would be compelling; also this would ensure that confounding effects related to known imprinted genes nearby were minimized). It was further decided that having one candidate with an ortholog predicted to be imprinted in the mouse but the other not was desirable to emphasize that the two sets of predictions did not overlap significantly and that novel human imprinted genes could be discovered even without relying on any conservation of imprinting status between human and mouse.

This approach resulted in a high-priority list of five genes. Conceptuses were screened to determine whether for each gene a sufficient number possessed an informative genotype that would permit experimental detection of monoallelic expression. The list was further narrowed to DLGAP2 and KCNK9, for which a detailed validation of imprinting status was undertaken.

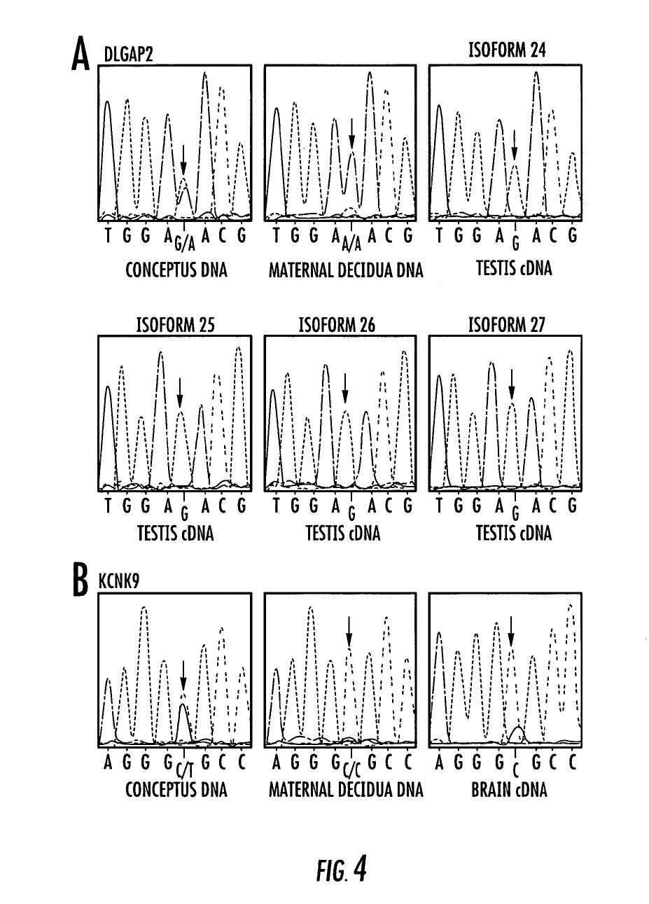

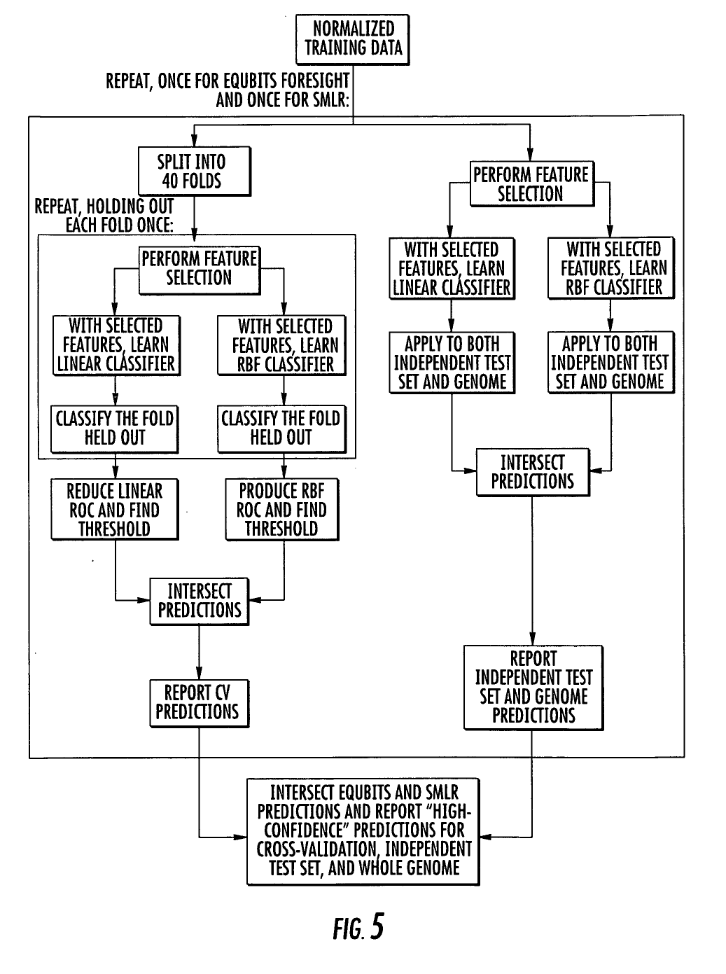

DLGAP2 is highly expressed and alternatively spliced in brain and testis (Ranta et al., 2000). It is contained within a 1.1 Mb interval on chromosome 8p23.3 that is frequently deleted in bladder cancer (Muscheck et al., 2000), making it a candidate tumor suppressor. cDNA containing polymorphic sites was generated by reverse transcription of total RNA isolated from brain and testis in heterozygous human conceptuses (N=8; gestational age: 63-105 days). The four isoforms of DLGAP2 (splice variants 24, 25, 26, and 27) (Karolchik et al., 2003) were paternally expressed in the testis of all samples (FIG. 4A) with some evidence of imprinting relaxation in isoforms 24 and 26. In contrast, expression from both alleles was observed for all four isoforms of DLGAP2 in whole brain. PEG1-AS is another imprinted gene predominantly expressed in the testis, and like DLGAP2 is expressed only from the paternal allele (Li et al., 2002).

KCNK9 resides at chromosomal location 8q24.3. It encodes the TASK3 (Twik-like acid-sensitive K+) channel and is predominantly expressed in the cerebellum (Medhurst et al., 2001). Therefore, RNA was isolated from the brains of conceptuses that were polymorphic at this locus (N=9; gestational age: 63-98 days). KCNK9 was exclusively expressed from the maternal allele in all samples (FIG. 4B). Thus, both genes chosen for experimental verification of their predicted imprint status were shown to be monoallelically expressed from the predicted parental allele (see Table 1 hereinbelow).

### Discussion of the Examples

Comparison to mouse. When making predictions with a classifier, it is preferable to weigh the trade-off between sensitivity and specificity, or analogously, between false positive rate and false negative rate. In the co-inventors' previous mouse study (Luedi et al., 2005), a greater focus was placed on keeping the false negative rate low. In the present human study, however, it was sought to keep the false positive rate low, defining the set of high confidence imprinted gene candidates as the intersection of four different classifiers. At least in part because of these different methodological choices, the number of imprinted genes predicted in the mouse and the number of high-confidence imprinted genes predicted in the human are not directly comparable. If a similar statistical methodology is adopted in the human as was used in the mouse, the number of human imprinted gene candidates increases, but is still only a little more than half as large as the mouse set. While these numbers are still not directly comparable since the sequence features in the human data are slightly richer than those in mouse, they are suggestive that the overall prevalence of imprinted genes is lower in human than in mouse.

The concordance between the high-confidence human imprinted candidates and the predictions for their orthologs in mouse was also investigated. A murine ortholog was identified for 119 of the genes proved or predicted with high confidence to be imprinted in human. Only 39 (33%) of these genes are known or predicted to be imprinted in both species (see Table 6 hereinbelow). This fraction does not change significantly if the same prediction method that was used for the mouse is also applied to the human data. Hence, the lack of greater overlap is not solely due to differences in the statistical approach.

That there are high levels of discordance of imprinting status between mouse and human has been recognized previously (Morison et al., 2005; Monk et al., 2006). It has been speculated that mice might have expanded genomic imprinting in order for the placenta to accommodate a large litter size and shorter gestational period, which might require an increased conservation of maternal resources (Monk et al., 2006). In contrast, human pregnancies tend to be singletons and of longer gestational time, which alleviates evolutionary pressure on imprinted genes to preserve maternal resources. Hence, it seems plausible that relatively fewer genes would be imprinted and maternally expressed in human (predicted proportion of 56% versus 64% in mouse); this is also consistent with the lower prevalence predicted overall. Of course, it is not the desire of the present co-inventors to be bound by any particular theory of operation in this regard.

The observed difference in the imprint status of genes in mouse and human raises the possibility that despite their immense popularity as models of human disease, mice might not be an ideal choice for studying diseases resulting principally from the epigenetic deregulation of imprinted genes, or for assessing human risk from environmental factors that alter the epigenome.

Imprinting and development. Of the 146 genes with a systematic name that are proved or predicted with high confidence to be imprinted, 38% are associated with embryonic development (based on PubMed abstracts); this compares to 18% among a random set of 5000 autosomal genes predicted not to be imprinted (P<1.7×10⁻⁹). As one interesting example, the homeobox (HOX) genes play a key role in pre- and post-implantation development (Eun Kwon & Taylor, 2004; Moens & Selleri, 2006). 23% of the HOX genes were predicted to be imprinted (9 out of 39; P<2×10⁻¹⁶). Five of the high-confidence candidates are located in the HOXA cluster, two in each of the HOXB and HOXC clusters, and none in the HOXD cluster. Several imprinted genes are known to be regulated in mouse by the same Polycomb group proteins (Mager et al., 2003; Umlauf et al., 2004) that also regulate HOX expression (Bantignies & Cavalli, 2006). Thus, there could be sequence characteristics shared in common between these two families of genes; however, no Hox genes were predicted to be imprinted in the mouse (Luedi et al., 2005). This indicates that the high prevalence of HOX imprinted gene candidates in human does not result simply from any shared sequence characteristics. Instead, it raises the possibility that monoallelic expression of HOX genes may have influenced human evolution, particularly the evolution of the brain.

Insights into the evolution of imprinting. Interestingly, recombination data was found to be of considerable importance for discriminating imprinted from non-imprinted genes. For example, an 8 basepair (bp) motif within THE1B elements that is overrepresented near recombination hotspots (Myers et al., 2005) is positively correlated with the presence of imprinted genes. In addition, the average distance between recombination hotspots and known imprinted genes is found to be about one third of that for all annotated genes. These observations lend support to the hypothesis that imprinted genes were originally linked in a few chromosomal regions, and were dispersed throughout the genome by recombination events during mammalian evolution (Walter & Paulsen, 2003). Of course, it is not the desire of the present co-inventors to be bound by any particular theory of operation in this regard.

In a cross-species comparison of imprinted regions between mouse and human, it has also been hypothesized that genomic imprinting might have evolved on the basis of dosage compensation following large-scale duplication events (Walter & Paulsen, 2003). To investigate this, it was asked whether the imprinted gene candidates were more likely to have been duplicated than the rest of the autosome. When using FASTA (Pearson & Lipman, 1988) to query each protein sequence against all other human proteins in our set, the distribution of the significance value for the second best hit was not different among imprinted gene candidates compared to the rest of the autosomal genes. Also, the proportion of paralogs that are located on the same chromosome was found not to differ between the two classes of genes, nor was there a significant difference in distance to that paralog. In conclusion, these findings fail to corroborate the hypothesis of large-scale gene duplication as the driving force of imprinting evolution. Of course, it is not the desire of the present co-inventors to be bound by any particular theory of operation in this regard.

Other hypotheses for the evolution of genomic imprinting include the proposition that imprinting is a by-product of a host defense against foreign DNA (Barlow, 1993; Yoder et al., 1997), or that during retrotransposition of a gene some regulatory elements may have been carried along with it that confer imprinted expression (Walter & Paulsen, 2003). To investigate this, it was determined whether the set of imprinted gene candidates identified was enriched for single-exon genes that might have been derived from multiexonic precursor paralogs. No significant difference in the rate of imprinted gene candidates consisting of only a single exon was observed compared to the autosomal genes not predicted to be imprinted (18% versus about 16%). Contrary to the observation that almost all known imprinted genes derived from retrotransposition are paternally expressed (Walter & Paulsen, 2003; Morison et al., 2005), it was also found that there was no statistically significant difference in the rate of intron-less genes among imprinted gene candidates with predicted maternal versus paternal expression. Of course, it is not the desire of the present co-inventors to be bound by any particular theory of operation in this regard.

Relevance for disease etiology. Parent-of-origin inheritance is increasingly observed in complex human health conditions such as alcoholism, Alzheimer's, asthma, autism, bipolar disorder, cancer, and schizophrenia (Murphy & Jirtle, 2003), providing evidence that imprinted genes play a role in their etiology. Furthermore, evidence is mounting for an association of assisted reproductive technology with birth defects and diseases caused by epigenetic dysregulation (Niemitz & Feinberg, 2004), which mostly involve imprinted genes. Disclosed herein is the successful mapping of genes proved or predicted with high confidence to be imprinted into chromosomal regions linked to a number of these complex conditions (see Table 7 hereinbelow). Interestingly, when candidate imprinted genes were mapped onto the overall human disease landscape defined by linkage analysis, some imprinted genes appeared to be involved in the etiology of multiple human diseases.

For example, KCNK9 is associated with a variety of human cancers (Patel & Lazdunski, 2004). It also resides at chromosome location 8q24 within 6 Mb of the marker D8S256 that is linked with bipolar disorder (McInnis et al., 2003; see Table 7 hereinbelow). Furthermore, since KCNK9 encodes for a potassium ion channel that mediates neuronal excitability, it is a strong candidate for idiopathic absence epilepsies (Zara et al., 1995; Kananura et al., 2002).

TABLE 1
High-confidence Imprinted Human Gene Candidates

| Ensembl ID | Band | Pred. |
|---|---|---|
| 184163 (Q5EBL5) | 1p36.33 | M |
| 107404 (DVL1) | 1p36.33 | M |
| 178821 (TMEM52) | 1p36.33 | P |
| 157911 (PEX10) | 1p36.32 | M |
| 177121 (Q8N6L5) | 1p36.32 | P |
| 142611 (PRDM16) | 1p36.32 | P |
| 116213 (WDR8) | 1p36.32 | M |
| 179163 (FUCA1) | 1p36.11 | P |
| 183682 (BMP8) | 1p34.3 | P |
| 173935 | 1p34.2 | M |
| (NM_182518) | | |
| 178973 | 1p34.2 | M |
| (NM_024547) | | |
| 137944 | 1p22.2 | M |
| (NM_019610) | | |
| 162676 (GF11) | 1p22.1 | P |
| 186371 (NDUFA4) | 1p13.3 | P |
| 173110 (HSPA6) | 1q23.3 | M |
| 152104 (PTPN14) | 1q32.3 | M |
| 124860 (OBSCN) | 1q42.13 | P |
| 181203 | 1q42.13 | M |
| (HIST3H2BB) | | |
| 177356 (Q8NGX0) | 1q44 | P |
| 138061 (CYP1B1) | 2p22.2 | P |
| 152518 (ZFP36L2) | 2p21 | M |
| 143921 (ABCG8) | 2p21 | M |
| 055813 (Q96PX6) | 2p16.1 | P |
| 115507 (OTX1) | 2p15 | M |
| 116035 (VAX2) | 2p13.3 | M |
| 169636 | 2q12.3 | P |
| 184764 (RPL22) | 2q13 | P |
| 171567 (TIGD1) | 2837.1 | P |
| 186540 (Q9Y419) | 2q37.3 | M |
| 172428 (MYEOV2) | 2q37.3 | P |
| 144908 (FTHFD) | 3q21.3 | M |
| 181882 | 3q22.3 | P |
| 152977 (ZIC1) | 3q24 | M |
| 114315 (HES1) | 3q29 | P |
| 127418 (FGFRL1) | 4p16.3 | M |
| 159674 (SPON2) | 4p16.3 | P |
| 163945 | 4p16.3 | M |
| (NP_065945.1) | | |
| 153851 (Q9NY19) | 4q13.2 | P |
| 153852 (Q9NYJ6) | 4q13.2 | P |
| 186158 | 4q35.2 | M |
| 186147 (DUX2) | 4q35.2 | P |
| 145536 | 5p15.32 | M |
| (ADAMTS16) | | |
| 145526 (CDH18) | 5p14.3 | P |
| 174132 (Q8TBP5) | 5q21.1 | P |
| 164400 (CSF2) | 5q23.3 | M |
| 145945 (FAM50B) | 6p25.2 | M |
| 168426 (BTNL2) | 6p21.32 | M |
| 135324 (C6orf117) | 6q14.2 | P |
| 112499 | 6q25.3 | P |
| (SLC22A2) | | |
| 060762 (BRP44L) | 6q27 | P |
| 105996 (HOXA2) | 7p15.2 | M |
| 105997 (HOXA3) | 7p15.2 | M |
| 106001 (HOXA4) | 7p15.2 | M |
| 106004 (HOXA5) | 7p15.2 | M |
| 005073 (HOXA11) | 7p15.2 | M |
| 106038 (EVX1) | 7p15.2 | P |
| 106571 (GLI3) | 7p14.1 | M |
| 185037 | 7q11.21 | M |
| 185947 (Q81VV5) | 7q11.21 | P |
| 135211 (C7orf35) | 7q11.23 | P |
| 187391 (MAG12) | 7821.11 | M |
| 164889 (SLC4A2) | 7q36.1 | M |
| 164896 (FASTK) | 7q36.1 | M |
| 180204 | 8p23.3 | P |
| (NM_181648) | | |
| 104284 (DLGAP2) | 8p23.3 | P |
| 185161 (Q8N914) | 8p23.1 | P |
| 172733 (PURG) | 8p12 | P |
| 167912 (Q96QE0) | 8q12.1 | M |
| 185942 (FAM77D) | 8q12.3 | P |
| 169427 (KCNK9) | 8q24.3 | M |
| 167656 (LY6D) | 8q24.3 | P |
| 167701 (GPT) | 8q24.3 | M |
| 186758 (Q8N710) | 9p21.1 | M |
| 107282 (APBA1) | 9821.11 | P |
| 155621 | 9q21.12 | P |
| (NM_182505) | | |
| 186788 | 9q21.32 | M |
| (NP_001001670) | | |
| 177945 | 9q33.3 | P |
| (NM_016158) | | |
| 136944 (LMX1B) | 9q33.3 | M |
| 160345 | 9q34.3 | P |
| (NM_144654) | | |
| 172889 (EGFL7) | 9q34.3 | P |
| 054148 (PHPT1) | 9q34.3 | M |
| 186909 | 10p15.3 | P |
| 107485 (GATA3) | 10p14 | P |
| 180740 (Q9H6Z8) | 10q23.31 | P |
| 148820 (LDB1) | 10q24.32 | M |
| 180066 (C10orf91) | 10q26.3 | M |
| 148826 (NKX6-2) | 10q26.3 | M |
| 171811 (C10orf93) | 10q26.3 | M |
| 151650 (VENTX2) | 10q26.3 | M |
| 178592 (Q8N377) | 10q26.3 | M |
| 148832 (PAOX) | 10q26.3 | M |
| 185885 (IFITM1) | 11p15.5 | M |
| 182272 | 11p15.5 | M |
| (B4GALNT4) | | |
| 184363 (PKP3) | 11p15.5 | M |
| 176828 (Q8N9U2) | 11p15.5 | M |
| 184682 | 11p15.5 | M |
| 184193 (Q8N7V1) | 11p14.3 | M |
| 174903 (RAB1B) | 11q13.2 | M |
| 182359 (KBTBD3) | 11q22.3 | P |
| 182657 | 11q24.3 | M |
| 182667 (NTR1) | 11q25 | P |
| 139194 (RBP5) | 12p13.31 | P |
| 069431 (ABCC9) | 12p12.1 | M |
| 180806 (HOXC9) | 12q13.13 | M |
| 186426 (HOXC4) | 12q13.13 | M |
| 135502 | 12q13.3 | M |
| (SLC26A10) | | |
| 135446 (CDK4) | 12q14.1 | M |
| 165891 (Q96AV8) | 12q21.2 | M |
| 112787 (Q9HCM7) | 12q24.33 | M |
| 178215 (Q8N7V5) | 13q21.1 | M |
| 177527 (Q8N7F4) | 13q21.31 | P |
| 185498 | 13q21.32 | P |
| 184497 (FAM70B) | 13q34 | M |
| 176165 (FOXG1C) | 14q12 | P |
| 073712 | 14q22.1 | P |
| (PLEKHCI) | | |
| 183992 | 14q31.1 | M |
| 185469 (RTL1) | 14q32.31 | M |
| 126290 (HV2A) | 14q32.33 | P |
| 151802 (Q9P068) | 15q13.1 | P |
| 005513 (SOX8) | 16p13.3 | P |
| 172268 (Q96S05) | 16p13.3 | P |
| 103449 (SALL0) | 16q12.1 | M |
| 103005 (C06orf57) | 16q13 | M |
| 102977 (ACD) | 16q22.1 | M |
| 103241 (FOXF1) | 16q24.1 | M |
| 183788 (Q8N206) | 16q24.3 | M |
| 183518 | 17p13.3 | M |
| 167874 (TMEM88) | 17p13.1 | M |
| 181977 (PYY2) | 17q11.2, | P |
| 173917 (HOXB2) | 17q21.32 | M |
| 120093 (HOXB3) | 17q21.32 | M |
| 141378 (YCE7) | 17q23.2 | M |
| 181428 (Q8N8L1) | 17q25.3 | P |
| 141441 (FAM59A) | 18q12.1 | P |
| 101489 | 18q12.2 | M |
| (BRUNOL4) | | |
| 141934 (PPAP2C) | 19p13.3 | M |
| 180866 (Q8NB05) | 19p13.2 | P |
| 172684 (Q8NE65) | 19p13.11 | P |
| 172666 | 19p13.11 | P |
| 121297 (TSH3) | 19q12 | P |
| 124302 (CHST8) | 19q13.11 | M |
| 180458 (Q8N3U1) | 19q13.13 | P |
| 159904 (ZNF225) | 19q13.31 | P |
| 167383 (ZNF229) | 19q13.31 | M |
| 186818 (LILRB4) | 19q13.42 | M |
| 105132 (ZN550) | 19q13.43 | M |
| 130724 (CHMP2A) | 19q13.43 | M |
| 099326 (ZNF42) | 19q13.43 | M |
| 101230 (C20orf82) | 20p12.1 | P |
| 101189 (C20orf20) | 20q13.33 | M |
| 092758 (COL9A3) | 20q13.33 | M |
| 159263 (SIM2) | 21q22.13 | P |
| 183628 (DGCR6) | 22q11.21 | M |
| 183099 | 22q11.21 | M |
| 184390 (Q61CM0) | 22q12.2 | P |
| 184687 (Q8ND38) | 22q13.31 | P |

The table lists high-confidence novel predictions of the combined classifier. Genes predicted to be expressed from the maternal or paternal allele are denoted by M or P, respectively. To enhance legibility, the common prefix “ENSG00000” has been dropped from the Ensembl ID. Also listed are gene names and/or GENBANK® Accession Nos. where applicable.

TABLE 2
High- and Lower-Confidence Imprinted Gene Candidates

| | | |
|---|---|---|
| Ensembl ID | Band | Pred.^{†} |

| | | | |
|---|---|---|---|
| 173447 | 1p36.33 | S | M |
| 184235 | 1p36.33 | S | M |
| 131591 | 1p36.33 | S | M |
| (NM_017891) | | | |
| 182839 | 1p36.33 | E | M |
| 184163 | 1p36.33 | E, S | M |
| (Q5EBL5) | | | |
| 131584 | 1p36.33 | S | M |
| (CENTB5) | | | |
| 127054 | 1p36.33 | S | M |
| (NM_017871) | | | |
| 169962 | 1p36.33 | S | M |
| (TAS/R3) | | | |
| 107404 (DVL1) | 1p36.33 | E, S | M |
| 162576 | 1p36.33 | S | M |
| (NM_032348) | | | |
| 160075 | 1p36.33 | S | M |
| (NM_014488) | | | |
| 178821 | 1p36.33 | E, S | P |
| (TMEM52) | | | |
| 157916 (RER1) | 1p36.33 | S | P |
| 157911 | 1p36.32 | E, S | M |
| (PEX10) | | | |
| 157881 | 1p36.32 | S | M |
| (PANK4) | | | |
| 169797 | 1p36.32 | S | M |
| 157870 | 1p36.32 | S | M |
| (NM_152371) | | | |
| 177121 | 1p36.32 | E, S | P |
| (Q8N6L5) | | | |
| 142611 | 1p36.32 | E, S | P |
| (PRDM16) | | | |
| 162591 | 1p36.32 | S | P |
| (EGFL3) | | | |
| 182956 | 1p36.32 | S | M |
| 116213 | 1p36.32 | E, S | M |
| (WDR8) | | | |
| 183509 | 1p36.32 | S | M |
| (Q8IYL3) | | | |
| 131697 | 1p36.31 | S | M |
| (Q9UFQ2) | | | |
| 130940 | 1p36.22 | S | M |
| (NM_017766) | | | |
| 117154 | 1p36.13 | S | P |
| (NM_032880) | | | |
| 179002 | 1p36.13 | S | P |
| (TASIR2) | | | |
| 179163 | 1p36.11 | E, S | P |
| (FUCA1) | | | |
| 142698 | 1p35.1 | E | P |
| (NM_032884) | | | |
| 126070 | 1p34.3 | E | M |
| (EIF2C3) | | | |
| 185668 | 1p34.3 | S | P |
| (POU3F1) | | | |
| 183682 (BMP8) | 1p34.3 | E, S | P |
| 173935 | 1p34.2 | E, S | M |
| (NM_182518) | | | |
| 178973 | 1p34.2 | E, S | M |
| (NM_024547) | | | |
| 117410 | 1p34.1 | S | M |
| (ATP6V0B) | | | |
| 118473 | 1p31.2 | E | P |
| (SG1P1) | | | |
| 132489 | 1p31.2 | E | P |
| (NM_020948) | | | |
| 117069 (S17E) | 1p31.1 | E | P |
| 137944 | 1p22.2 | E, S | M |
| (NM_019610) | | | |
| 162676 (GF11) | 1p22.1 | E, S | P |
| 182166 | 1p21.2 | S | P |
| 186371 | 1p13.3 | E, S | P |
| (NDUFA4) | | | |
| 121931 | 1p13.3 | S | P |
| (NM_018372) | | | |
| 116455 (ME50) | 1p13.2 | S | P |
| 179735 | 1q21.1 | S | P |
| (Q8NE92) | | | |
| 184458 | 1q21.3 | S | P |
| (Q86YZ3) | | | |
| 169474 | 1q21.3 | S | M |
| (SPRR1A) | | | |
| 160691 (SHC1) | 1q22 | S | M |
| 143620 | 1q22 | S | M |
| (EFNA4) | | | |
| 160856 | 1q23.1 | S | P |
| (NM_052939) | | | |
| 132704 | 1q23.1 | E | M |
| (FCRL2) | | | |
| 132703 (APCS) | 1q23.2 | S | P |
| 173110 | 1q23.3 | E, S | M |
| (HSPA6) | | | |
| 143152 | 1q24.1 | S | M |
| (Q9C074) | | | |
| 117501 | 1q24.3 | S | P |
| (NM_025063) | | | |
| 116147 (TNR) | 1q25.1 | S | P |
| 116703 (PDC) | 1q31.1 | S | P |
| 118194 | 1832.1 | S | P |
| (TNNT2) | | | |
| 152104 | 1q32.3 | E, S | M |
| (PTPN14) | | | |
| 152120 | 1q41 | S | P |
| (Q9NQ13) | | | |
| 117791 | 1q41 | S | P |
| (NM_017898) | | | |
| 185495 | 1q42.1 | 1S | M |
| (Q9H5Q3) | | | |
| 173419 | 1q42.12 | S | P |
| (Q8IVP0) | | | |
| 081692 | 1q42.13 | S | P |
| (NM_023007) | | | |
| 124860 | 1q42.13 | E, S | P |
| (OBSCN) | | | |
| 181203 | 1q42.13 | E, S | M |
| (HIST3H2BB) | | | |
| 168159 | 1q42.13 | E | M |
| (Q5TA31) | | | |
| 182887 | 1q42.13 | E | P |
| 162946 (DISC1) | 1q42.2 | S | M |
| 179397 | 1q44 | S | M |
| (NM_173807) | | | |
| 177356 | 1q44 | E, S | P |
| (Q8NGX0) | | | |
| 035115 | 2p25.3 | S | M |
| (NM_015677) | | | |
| 172554 | 2p25.3 | S | P |
| (SNTG2) | | | |
| 186170 | 2p25.3 | S | M |
| (TMSL2) | | | |
| 182551 | 2p25.2 | S | P |
| (NM_018269) | | | |
| 134321 | 2p25.2 | S | P |
| (NM_080657) | | | |
| 115738 (JD2) | 2p25.1 | E | M |
| 138061 | 2p22.2 | E, S | P |
| (CYP1B1) | | | |
| 152154 | 2p22.1 | S | P |
| (NM_152390) | | | |
| 152518 | 2p21 | E, S | M |
| (ZFP36L2) | | | |
| 143921 | 2p21 | E, S | M |
| (ABCG8) | | | |
| 138083 (SIX3) | 2p21 | S | P |
| 055813 | 2p16.1 | E, S | P |
| (Q96PX6) | | | |
| 115507 (OTX1) | 2p15 | E, S | M |
| 116035 (VAX2) | 2p13.3 | E, S | M |
| 178455 | 2p13.2 | S | P |
| 003137 (C26A) | 2p13.2 | S | P |
| 144040 | 2p13.2 | S | P |
| (SFXN5) | | | |
| 135637 | 2p13.1 | S | M |
| (MRPL53) | | | |
| 115325 (DOK1) | 2p13.1 | S | M |
| 116119 (KV2A) | 2p11.2 | S | P |
| 115085 | 2q11.2 | S | P |
| (ZAP70) | | | |
| 135951 | 2q11.2 | E | P |
| (TSGA10) | | | |
| 071082 | 2q11.2 | S | P |
| (RPL31) | | | |
| 169636 | 2812.3 | E, S | P |
| 183998 | 2q13 | E | P |
| (RPL22) | | | |
| 015568 | 2q13 | S | P |
| (RANBP2L1) | | | |
| 184764 | 2q13 | E, S | P |
| (RPL22) | | | |
| 184538 | 2q13 | S | P |
| (RANBP2L1) | | | |
| 153094 | 2q13 | S | P |
| (BCL2L11) | | | |
| 125618 (PAX8) | 2q13 | S | M |
| 183300 | 2q14.3 | S | P |
| 136720 | 2q14.3 | S | P |
| (HS6ST1) | | | |
| 169822 | 2q14.3 | S | P |
| (NM_030970) | | | |
| 136698 | 2q21.1 | S | M |
| (NM_032545) | | | |
| 179843 | 2q21.1 | S | M |
| (RAB6C) | | | |
| 183840 | 2q21.2 | E | M |
| (GPR39) | | | |
| 136539 | 2q24.2 | S | P |
| (NM_014880) | | | |
| 174470 | 2q24.2 | S | M |
| (Q96M44) | | | |
| 128714 | 2q31.1 | S | M |
| (HOXDJ3) | | | |
| 128713 | 2q31.1 | S | M |
| (HOXD11) | | | |
| 128709 | 2q31.1 | S | M |
| (HOXD9) | | | |
| 170166 | 2q31.1 | E | M |
| (HOXD4) | | | |
| 171567 (TIGD1) | 2q37.1 | E, S | P |
| 157985 | 2q37.2 | E | M |
| (CENTG2) | | | |
| 144485 (HES6) | 2q37.3 | S | M |
| 132326 (PER2) | 2q37.3 | S | M |
| 186540 | 2q37.3 | E, S | M |
| (Q9Y419) | | | |
| 178580 | 2q37.3 | S | P |
| (Q81YXC7) | | | |
| 172428 | 2q37.3 | E, S | P |
| (MYEOV2) | | | |
| 178602 | 2q37.3 | S | P |
| (NM_148961) | | | |
| 063660 (GPC1) | 2q37.3 | S | M |
| 142327 | 2q37.3 | S | M |
| (RNPEPL1) | | | |
| 115687 (PASK) | 2q37.3 | E | M |
| 132170 | 3p25.2 | S | P |
| (PPARG) | | | |
| 131374 | 3p24.3 | E | M |
| (TBC1D5) | | | |
| 060971 | 3p22.3 | S | P |
| (ACAA1) | | | |
| 010282 (KB73) | 3p22.1 | S | P |
| 178055 | 3p21.31 | S | M |
| (NM_182702) | | | |
| 068028 | 3p21.31 | S | M |
| (RASSF1) | | | |
| 145050 | 3p21.31 | S | P |
| (ARMET) | | | |
| 114841 | 3p21.1 | S | M |
| (NM_015512) | | | |
| 010322 | 3p21.1 | S | M |
| (NISCH) | | | |
| 168268 | 3p21.1 | S | M |
| (NM_022908) | | | |
| 144741 | 3p14.1 | S | P |
| (NM_173471) | | | |
| 183185 | 3q12.1 | S | P |
| (Q9UIV9) | | | |
| 184804 | 3q13.12 | E | M |
| 185565 | 3q13.31 | S | M |
| (LSAMP) | | | |
| 144908 | 3q21.3 | E, S | M |
| (FTHFD) | | | |
| 114626 | 3q21.3 | S | P |
| (ABTB1) | | | |
| 179348 | 3q21.3 | S | M |
| (GATA2) | | | |
| 004399 | 3q22.1 | S | P |
| (PLXND1) | | | |
| 174640 | 3q22.1 | S | P |
| (SLC21A2) | | | |
| 144872 | 3q22.2 | S | P |
| 181882 | 3q22.3 | E, S | P |
| 168875 | 3q22.3 | S | M |
| (SOX14) | | | |
| 114120 | 3q23 | S | M |
| (NM_018155) | | | |
| 175685 | 3q24 | S | P |
| (Q9BZ57) | | | |
| 174963 (ZIC4) | 3q24 | S | M |
| 152977 (ZIC1) | 3q24 | E, S | M |
| 175726 | 3q25.1 | S | M |
| 174948 | 3q25.2 | S | M |
| (Q86SP6) | | | |
| 151967 | 3q25.32 | S | P |
| (SCH1P1) | | | |
| 181501 | 3q26.33 | E | M |
| 163882 | 3q27.1 | S | M |
| (POLR2H) | | | |
| 114315 (HES1) | 3q29 | E, S | P |
| 169020 | 4p16.3 | S | M |
| (ATP51) | | | |
| 145214 (DGKQ) | 4p16.3 | S | M |
| 127418 | 4p16.3 | E, S | M |
| (FGFRL1) | | | |
| 176836 | 4p16.3 | S | P |
| 159674 | 4p16.3 | E, S | P |
| (SPON2) | | | |
| 163945 | 4p16.3 | E, S | M |
| (NP_065945.1) | | | |
| 174141 | 4p16.3 | S | P |
| (Q15270) | | | |
| 068078 | 4p16.3 | S | M |
| (FGFR3) | | | |
| 163956 | 4p16.3 | S | M |
| (LRPAP1) | | | |
| 183190 | 4p13 | E | M |
| 182739 | 4q13.2 | S | P |
| (GRINL1B) | | | |
| 153851 | 4q13.2 | E, S | P |
| (Q9NY19) | | | |
| 153852 | 4q13.2 | E, S | P |
| (Q9NY16) | | | |
| 180769 | 4q21.23 | S | M |
| (Q8N507) | | | |
| 138821 | 4q24 | S | P |
| (NM_022154) | | | |
| 168743 | 4q24 | E | P |
| (NP_001028219) | | | |
| 164093 (PITX2) | 4q25 | S | P |
| 177826 | 4q28.1 | S | P |
| 170153 | 4q31.21 | S | M |
| (Q9ULK6) | | | |
| 180519 | 4q31.21 | S | P |
| 151615 | 4q31.22 | S | M |
| (POU4F2) | | | |
| 172799 | 4q31.3 | S | M |
| 145431 | 4832.1 | E | P |
| (PDGFC) | | | |
| 038295 (TLL1) | 4q32.3 | S | P |
| 056050 | 4q33 | S | M |
| (NM_017867) | | | |
| 168322 | 4q34.3 | S | P |
| (NM_030970) | | | |
| 177310 | 4q35.1 | E | M |
| (NM_153008) | | | |
| 186158 | 4q35.2 | E, S | M |
| 186147 (DUX2) | 4q35.2 | E, S | P |
| 066230 | 5p15.33 | S | M |
| (SLC9A3) | | | |
| 185486 | 5p15.33 | S | M |
| 125063 | 5p15.33 | S | M |
| (NM_017808) | | | |
| 112877 | 5p15.33 | S | M |
| (NM_018140) | | | |
| 145506 (NKD2) | 5p15.33 | S | M |
| 113504 | 5p15.33 | S | M |
| (SLC12A7) | | | |
| 174358 | 5p15.33 | S | M |
| 153395 | 5p15.33 | S | M |
| (Q8NF37) | | | |
| 113430 (IRX4) | 5p15.33 | S | M |
| 170561 (IRX2) | 5p15.33 | S | P |
| 170549 (IRX1) | 5p15.33 | S | M |
| 145536 | 5p15.32 | E, S | M |
| (ADAMTS16) | | | |
| 164236 | 5p15.2 | E | P |
| (XP_293937.5) | | | |
| 133357 | 5p15.2 | E | P |
| (NM_030970) | | | |
| 145526 | 5p14.3 | E, S | P |
| (CDH18) | | | |
| 132404 | 5p14.1 | S | P |
| 113492 | 5p13.2 | E | P |
| (AGXT2) | | | |
| 168621 (GDNF) | 5p13.2 | S | P |
| 016082 (ISL1) | 5q11.2 | S | P |
| 164258 | 5q11.2 | S | P |
| (NDUFS4) | | | |
| 164283 (ESM1) | 5q11.2 | S | P |
| 152929 | 5q12.1 | S | M |
| (Q9BXE3) | | | |
| 145645 | 5q12.1 | S | P |
| (Q9P193) | | | |
| 171540 (OTP) | 5q14.1 | S | M |
| 131730 | 5q14.1 | S | M |
| (CKMT2) | | | |
| 131732 | 5q14.1 | S | M |
| (NM_032280) | | | |
| 153922 (CHD1) | 5q15 | E | M |
| 174132 | 5q21.1 | E, S | P |
| (Q8TBP5) | | | |
| 181751 | 5q21.1 | S | M |
| (NM_033211) | | | |
| 176857 | 5q21.3 | E | M |
| 080709 | 5q22.3 | S | M |
| (KCNN2) | | | |
| 113396 | 5q23.3 | S | M |
| (SLC27A6) | | | |
| 164400 (CSF2) | 5q23.3 | E, S | M |
| 069011 (PITX1) | 5q31.1 | S | P |
| 174313 | 5q31.1 | E | P |
| 081818 | 5q31.3 | S | M |
| (PCDHB4) | | | |
| 177895 | 5q31.3 | S | P |
| (PCDHB16) | | | |
| 120327 | 5q31.3 | E | P |
| (PCDHB14) | | | |
| 081853 | 5q31.3 | S | M |
| (PCDHGC5) | | | |
| 113580 | 5q31.3 | E | P |
| (NR3C1) | | | |
| 169302 | 5q32 | S | P |
| 113667 (Y555) | 5q32 | E | M |
| 145888 | 5q33.1 | E | P |
| (GLRA1) | | | |
| 182344 | 5q35.2 | S | M |
| 185548 | 5q35.3 | S | M |
| 178392 | 5q35.3 | S | M |
| 185784 | 5q35.3 | E | M |
| (Q8TAJ0) | | | |
| 168903 | 5q35.3 | S | P |
| (BTNL3) | | | |
| 137273 | 6p25.3 | S | P |
| (FOXF2) | | | |
| 184250 | 6p25.2 | S | M |
| (Q86WA7) | | | |
| 145945 | 6p25.2 | E, S | M |
| (FAM50B) | | | |
| 124785 (NRN1) | 6p25.1 | S | M |
| 137203 | 6p24.3 | S | M |
| (TFAP2A) | | | |
| 176078 | 6p24.3 | S | M |
| (Q8NAN4) | | | |
| 185694 | 6p22.1 | E | P |
| 181573 | 6p22.1 | S | P |
| (Q96MM2) | | | |
| 112498 | 6p22.1 | S | M |
| (PPPIR11) | | | |
| 161877 | 6p21.32 | S | M |
| (C60orf10) | | | |
| 168426 | 6p21.32 | E, S | M |
| (BTNL2) | | | |
| 168383 (HLA- | 6p21.32 | E | P |
| DPB1) | | | |
| 161896 (IHPK3) | 6p21.31 | S | M |
| 156582 | 6p21.2 | S | M |
| 137252 | 6p12.1 | S | P |
| (HCRTR2) | | | |
| 146151 | 6p12.1 | S | P |
| (HMGCLL1) | | | |
| 179713 | 6q14.1 | S | P |
| (Q8N481) | | | |
| 135324 | 6q14.2 | E, S | P |
| (C6orf17) | | | |
| 135315 | 6q14.2 | S | P |
| (C6orf84) | | | |
| 184486 | 6q16.1 | S | M |
| (POU3F4) | | | |
| 183075 | 6q21 | S | P |
| 153989 | 6q22.1 | S | P |
| (C6orf68) | | | |
| 146350 | 6q22.31 | E | P |
| (C6orf170) | | | |
| 184362 | 6q22.31 | S | P |
| (Q9BZ63) | | | |
| 175211 | 6q23.2 | S | P |
| (Q9BXE6) | | | |
| 135521 | 6q24.2 | S | M |
| (C6orf93) | | | |
| 118508 | 6q24.3 | S | P |
| (RAB32) | | | |
| 112499 | 6q25.3 | E, S | P |
| (SLC22A2) | | | |
| 146477 | 6q25.3 | S | P |
| (SLC22A3) | | | |
| 060762 | 6q27 | E, S | P |
| (BRP44L) | | | |
| 153471 | 6q27 | S | P |
| (TCP10) | | | |
| 186340 | 6q27 | S | P |
| (THBS2) | | | |
| 164493 | 6q27 | S | P |
| (Q96N37) | | | |
| 170767 | 6q27 | E | M |
| (C6orf208) | | | |
| 177706 | 7p22.3 | S | P |
| (FAM20C) | | | |
| 184773 | 7p22.3 | E | M |
| (Q96GH9) | | | |
| 122691 | 7p21.1 | S | M |
| (TWIST2) | | | |
| 105855 (ITGB8) | 7p21.1 | E | M |
| 105996 | 7p15.2 | E, S | M |
| (HOXA2) | | | |
| 105997 | 7p15.2 | E, S | M |
| (HOXA3) | | | |
| 164519 | 7p15.2 | S | M |
| (Q96MZ3) | | | |
| 106001 | 7p15.2 | E, S | M |
| (HOXA4) | | | |
| 106004 | 7p15.2 | E, S | M |
| (HOXA5) | | | |
| 106006 | 7p15.2 | S | M |
| (HOXA6) | | | |
| 005073 | 7p15.2 | E, S | M |
| (HOXA11) | | | |
| 106038 (EVX1) | 7p15.2 | E, S | P |
| 106483 | 7p14.1 | S | P |
| (SFRP4) | | | |
| 106571 (GLI3) | 7p14.1 | E, S | M |
| 164543 | 7p13 | E | P |
| (STKJ7A) | | | |
| 058404 | 7p13 | S | P |
| (CAMK2B) | | | |
| 164742 | 7p13 | S | M |
| (ADCY1) | | | |
| 185292 | 7p13 | S | M |
| 179869 | 7p12.3 | S | P |
| (NM_152701) | | | |
| 042813 (ZPBP) | 7p12.2 | S | P |
| 185037 | 7q11.21 | E, S | M |
| 185947 | 7q11.21 | E, S | P |
| (Q81VV5) | | | |
| 135211 | 7q11.23 | E, S | P |
| (C7orf35) | | | |
| 187391 | 7q21.11 | E, S | M |
| (MAG12) | | | |
| 185191 | 7q21.12 | S | P |
| 182348 | 7q21.13 | S | P |
| (NM_181646) | | | |
| 105810 (CDK6) | 7q21.2 | S | M |
| 006377 (DLX6) | 7q21.3 | S | P |
| 121716 | 7q22.1 | S | M |
| (P1LRB) | | | |
| 128594 | 7q32.1 | S | P |
| (NM_022143) | | | |
| 106028 | 7q34 | S | M |
| (SSBP1) | | | |
| 181551 | 7q34 | S | P |
| 184412 | 7q34 | S | P |
| 133624 | 7q36.1 | S | P |
| (NM_024910) | | | |
| 164889 | 7q36.1 | E, S | M |
| (SLC4A2) | | | |
| 164896 | 7q36.1 | E, S | M |
| (FASTK) | | | |
| 164690 (SHH) | 7q36.3 | S | P |
| 187177 | 7q36.3 | E | M |
| 146909 (C7orf3) | 7q36.3 | E | P |
| 130675 | 7q36.3 | S | M |
| (HLXB9) | | | |
| 178158 | 7q36.3 | S | M |
| (Q8N7D3) | | | |
| 155093 | 7q36.3 | S | M |
| (PTPRN2) | | | |
| 180204 | 8p23.3 | E, S | P |
| (NM_181648) | | | |
| 104284 | 8p23.3 | E, S | P |
| (DLGAP2) | | | |
| 036448 | 8p23.3 | S | M |
| (MYOM2) | | | |
| 186550 | 8p23.1 | E | M |
| 186553 | 8p23.1 | E | M |
| 186555 | 8p23.1 | E | P |
| 186558 | 8p23.1 | E | P |
| 186560 | 8p23.1 | E | M |
| 186647 | 8p23.1 | E | M |
| 185161 | 8p23.1 | E, S | P |
| (Q8N9J4) | | | |
| 158815 | 8p21.3 | S | M |
| (FGF17) | | | |
| 168487 (BMP1) | 8p21.3 | S | P |
| 120896 (VINE) | 8p21.3 | S | M |
| 179388 (EGR3) | 8p21.3 | S | M |
| 172733 (PURG) | 8p12 | E, S | P |
| 167912 | 8q12.1 | E, S | M |
| (Q96QE0) | | | |
| 183226 | 8q12.3 | E | P |
| 185942 | 8q12.3 | E, S | P |
| (FAM77D) | | | |
| 165084 | 8q13.2 | E | M |
| (NM_052958) | | | |
| 184234 | 8q21.2 | S | M |
| (NM_172239) | | | |
| 180694 | 8q21.3 | S | P |
| (Q8N3G6) | | | |
| 156486 | 8q22.2 | S | P |
| (KCNS2) | | | |
| 164796 | 8q23.3 | S | M |
| (CSMD3) | | | |
| 104406 | 8q24.22 | E | P |
| (NM_032205) | | | |
| 169427 | 8q24.3 | E, S | M |
| (KCNK9) | | | |
| 184489 | 8q24.3 | S | P |
| (PTP4A3) | | | |
| 181790 (BAI1) | 8q24.3 | S | M |
| 180838 | 8q24.3 | E | M |
| (Q8NAM3) | | | |
| 167656 (LY6D) | 8q24.3 | E, S | P |
| 179142 | 8q24.3 | S | M |
| (CYP11B2) | | | |
| 182851 | 8q24.3 | E | P |
| (NM_178172) | | | |
| 158106 | 8q24.3 | S | M |
| (RHPN1) | | | |
| 181528 | 8q24.3 | S | M |
| 179950 | 8q24.3 | S | P |
| (NM_078480) | | | |
| 185189 | 8q24.3 | S | M |
| (NM_178564) | | | |
| 186574 | 8q24.3 | S | M |
| (Q8ND02) | | | |
| 178719 | 8q24.3 | E | P |
| (GRINA) | | | |
| 167701 (GPT) | 8q24.3 | E, S | M |
| 160959 (YOJ4) | 8q24.3 | S | P |
| 177742 | 8q24.3 | E | M |
| (NM_178535) | | | |
| 120215 | 9p24.1 | S | M |
| (MLANA) | | | |
| 186758 | 9p21.1 | E, S | M |
| (Q8N710) | | | |
| 174994 | 9p12 | S | P |
| (Q96M55) | | | |
| 170152 | 9p11.2 | S | M |
| 154537 | 9p11.2 | S | M |
| (Q8NCQ8) | | | |
| 178784 | 9q12 | S | P |
| (Q96F02) | | | |
| 184879 | 9q13 | S | M |
| 182368 | 9q13 | S | M |
| (Q8NCQ8) | | | |
| 170215 | 9q13 | S | M |
| (Q8NCQ8) | | | |
| 170217 | 9q13 | S | M |
| 107282 | 9q21.11 | E, S | P |
| (APBA1) | | | |
| 155621 | 9q21.12 | E, S | P |
| (NM_182505) | | | |
| 186788 | 9q21.32 | E, S | M |
| (NP_001001670) | | | |
| 177992 | 9q22.1 | S | P |
| (NM_178828) | | | |
| 186359 | 9q22.1 | S | P |
| (Q8NDSJ) | | | |
| 130222 | 9q22.2 | S | P |
| (GADD45G) | | | |
| 169027 | 9q22.31 | S | P |
| (NM_030970) | | | |
| 131662 (PHF2) | 9q22.31 | S | P |
| 119523 | 9q22.33 | S | P |
| (NM_033087) | | | |
| 177945 | 9q33.3 | E, S | P |
| (NM_016158) | | | |
| 136944 (LMXIB) | 9q33.3 | E, S | M |
| 123454 (DBH) | 9q34.2 | S | M |
| 186459 | 9q34.3 | S | P |
| 160345 | 9q34.3 | E, S | P |
| (NM_144654) | | | |
| 148411 | 9q34.3 | S | M |
| (NM_144653) | | | |
| 160360 | 9q34.3 | S | M |
| (Q9UFS8) | | | |
| 148400 | 9q34.3 | S | M |
| (NOTCH1) | | | |
| 172889 | 9q34.3 | E, S | P |
| (EGFL7) | | | |
| 169692 | 9q34.3 | S | M |
| (AGPAT2) | | | |
| 054148 | 9q34.3 | E, S | M |
| (PHPT1) | | | |
| 184709 | 9q34.3 | S | M |
| 185863 | 9q34.3 | S | M |
| 176248 | 9q34.3 | S | M |
| (NM_013366) | | | |
| 176058 | 9q34.3 | S | M |
| (NM_173691) | | | |
| 182569 | 9q34.3 | S | M |
| (NM_053045) | | | |
| 186909 | 10p15.3 | E, S | P |
| 151632 | 10p15.1 | S | P |
| (AKR1C2) | | | |
| 178462 | 10p15.1 | S | M |
| (NM_024803) | | | |
| 178372 (CLSP) | 10p15.1 | S | P |
| 176730 | 10p15.1 | E | M |
| (Q8N218) | | | |
| 107485 | 10p14 | E, S | P |
| (GATA3) | | | |
| 182077 | 10p12.1 | E | M |
| (NP_001030014) | | | |
| 099250 (NRP1) | 10p11.22 | E | P |
| 175395 | 10p11.21 | E | M |
| (ZNF25) | | | |
| 165511 | 10q11.21 | S | M |
| (NM_145022) | | | |
| 165406 | 10q11.21 | E | P |
| (MARCH8) | | | |
| 148611 | 10q11.22 | S | M |
| (SYT15) | | | |
| 165606 | 10q11.23 | S | M |
| 107671 | 10q11.23 | S | M |
| (NM_018245) | | | |
| 165443 | 10q21.1 | S | M |
| (NM_032439) | | | |
| 148575 | 10q21.2 | S | M |
| (NM_178505) | | | |
| 182771 (GRID1) | 10q23.2 | E | M |
| 138135 | 10q23.31 | S | P |
| (CH25H) | | | |
| 180740 | 10q23.31 | E, S | P |
| (Q9H6Z8) | | | |
| 095585 (BLNK) | 10q24.1 | S | P |
| 148820 (LDB1) | 10q24.32 | E, S | M |
| 166275 | 10q24.32 | S | P |
| (NM_144591) | | | |
| 176584 | 10q26.13 | S | M |
| 119965 | 10q26.13 | E | M |
| (C10orf88) | | | |
| 108001 (EBF3) | 10q26.3 | S | M |
| 165752 | 10q26.3 | S | P |
| (NM_173575) | | | |
| 171813 | 10q26.3 | S | P |
| (Q96F43) | | | |
| 180066 | 10q26.3 | E, S | M |
| (C10orf91) | | | |
| 148826 | 10q26.3 | E, S | M |
| (NKX6-2) | | | |
| 171811 | 10q26.3 | E, S | M |
| (C10orf93) | | | |
| 151646 | 10q26.3 | S | M |
| (GPR123) | | | |
| 171798 | 10q26.3 | S | M |
| (Q8TEE5) | | | |
| 165824 | 10q26.3 | S | M |
| (NM_152643) | | | |
| 171794 (UTF1) | 10q26.3 | S | P |
| 151650 | 10q26.3 | E, S | M |
| (VENTX2) | | | |
| 178592 | 10q26.3 | E, S | M |
| (Q8N377) | | | |
| 148832 (PAOX) | 10q26.3 | E, S | M |
| 186730 (DUX4) | 10q26.3 | S | P |
| 184243 | 10q26.3 | S | P |
| 179882 (DUX2) | 10q26.3 | S | P |
| 177947 (ODF3) | 11p15.5 | S | M |
| 174885 (PYA5) | 11p15.5 | S | M |
| 185885 | 11p15.5 | E, S | M |
| (IFITM1) | | | |
| 182272 | 11p15.5 | E, S | M |
| (B4GALNT4) | | | |
| 184363 (PKP3) | 11p15.5 | E, S | M |
| 176828 | 11p15.5 | E, S | M |
| (Q8N9U2) | | | |
| 177700 | 11p15.5 | S | M |
| (POLR2L) | | | |
| 184956 (MUC6) | 11p15.5 | S | M |
| 183116 | 11p15.5 | S | M |
| 184545 | 11p15.5 | S | P |
| (DUSP8) | | | |
| 130598 | 11p15.5 | S | P |
| (TNN12) | | | |
| 184682 | 11p15.5 | E, S | M |
| 183680 | 11p15.5 | S | P |
| (Q8N2L8) | | | |
| 181963 | 11p15.4 | S | P |
| (Q8NGK3) | | | |
| 180785 | 11p15.4 | S | M |
| (NM_152430) | | | |
| 176904 | 11p15.4 | S | P |
| (Q8NH63) | | | |
| 180974 | 11p15.4 | S | P |
| (Q8NGH9) | | | |
| 051009 | 11p15.4 | S | M |
| (NM_032127) | | | |
| 166337 (TAF10) | 11p15.4 | S | P |
| 170748 | 11p15.4 | S | P |
| (NM_14469) | | | |
| 170688 | 11p15.4 | E | P |
| (OR5EJP) | | | |
| 129152 | 11p15.1 | S | M |
| (MYOD1) | | | |
| 184193 | 11p14.3 | E, S | M |
| (Q8N7V1) | | | |
| 129151 | 11p14.2 | E | P |
| (BBOX1) | | | |
| 007372 (PAX6) | 11p13 | S | M |
| 183242 (WIT1) | 11p13 | S | M |
| 182565 | 11p11.12 | S | P |
| 185927 | 11q11 | S | P |
| 186660 (ZFP91) | 11q12.1 | S | P |
| 172289 | 11q12.1 | S | P |
| (Q8NG17) | | | |
| 134824 | 11q12.2 | S | M |
| (FADS2) | | | |
| 174903 | 11q13.2 | E, S | M |
| (RAB1D) | | | |
| 174851 (YIF1) | 11q13.2 | S | M |
| 173621 | 11q13.2 | S | P |
| (NM_024036) | | | |
| 172932 | 11q13.2 | S | P |
| 162105 | 11q13.3 | S | M |
| (SHANK2) | | | |
| 175534 | 11q13.4 | S | M |
| (Q8TB74) | | | |
| 137474 | 11q13.5 | S | P |
| (MY07A) | | | |
| 168959 (GRM5) | 11q14.2 | S | P |
| 182359 | 11q22.3 | E, S | P |
| (KBTBD3) | | | |
| 150750 | 11q23.1 | E | M |
| (C11orf53) | | | |
| 184824 | 11q23.3 | S | M |
| (C1QTNF5) | | | |
| 154146 (NRGN) | 11q24.2 | S | P |
| 182657 | 11q24.3 | E, S | M |
| 120462 | 11q24.3 | S | M |
| (Q9P195) | | | |
| 182667 (NTR1) | 11q25 | E, S | P |
| 170257 | 11q25 | S | P |
| (NM_030970) | | | |
| 080854 | 11q25 | S | M |
| (Q9UPX0) | | | |
| 151503 (Y056) | 11q25 | S | M |
| 149328 | 11q25 | S | M |
| (NM_138342) | | | |
| 109956 | 11q25 | S | M |
| (B3GAT1) | | | |
| 139194 (RBP5) | 12p13.31 | E, S | P |
| 150045 | 12p13.31 | S | P |
| (KLRF1) | | | |
| 121374 | 12p13.2 | S | P |
| (KLRC3) | | | |
| 171681 | 12p13.1 | S | M |
| (ATF71P) | | | |
| 111404 | 12p12.3 | S | P |
| (NM_024730) | | | |
| 172572 | 12p12.2 | S | M |
| (PDE3A) | | | |
| 11700 | 12p12.2 | S | P |
| (SLC21A8) | | | |
| 069431 | 12p12.1 | E, S | M |
| (ABCC9) | | | |
| 013573 | 12p11.21 | S | M |
| (DDX11) | | | |
| 177627 | 12q13.11 | E | P |
| (NM_1523I9) | | | |
| 123364 | 12q13.13 | S | M |
| (HOXC13) | | | |
| 123388 | 12q13.13 | S | M |
| (HOXC11) | | | |
| 180818 | 12q13.13 | S | M |
| (HOXC10) | | | |
| 180806 | 12q13.13 | E, S | M |
| (HOXC9) | | | |
| 186426 | 12q13.13 | E, S | M |
| (HOXC4) | | | |
| 170338 | 12q13.13 | S | M |
| (HOXC6) | | | |
| 172789 | 12q13.13 | E | M |
| (HOXC5) | | | |
| 174604 | 12q13.2 | S | P |
| (Q9BXE6) | | | |
| 135502 | 12q13.3 | E, S | M |
| (SLC26A10) | | | |
| 135446 (CDK4) | 12q14.1 | E, S | M |
| 079081 | 12q14.2 | S | M |
| (SRGAP1) | | | |
| 173401 | 12q21.1 | E | M |
| (NM_152779) | | | |
| 165891 | 12q21.2 | E, S | M |
| (Q96AV8) | | | |
| 111046 (MYF6) | 12q21.31 | S | P |
| 151572 | 12q23.1 | S | P |
| (NM_178826) | | | |
| 089116 (LHX5) | 12q24.13 | S | M |
| 175727 | 12q24.31 | S | P |
| (NM_014938) | | | |
| 184967 | 12q24.33 | S | M |
| (NM_024078) | | | |
| 112787 | 12q24.33 | E, S | M |
| (Q9HCM7) | | | |
| 139495 | 13q12.12 | S | P |
| (NM_153023) | | | |
| 169840 (GSH1) | 13q12.2 | S | M |
| 102760 | 13q14.11 | S | M |
| (NM_014059) | | | |
| 152207 | 13q14.2 | S | P |
| (CYSLTR2) | | | |
| 171945 | 13q14.3 | S | P |
| (NM_030970) | | | |
| 178215 | 13q21.1 | E, S | M |
| (Q8N7V5) | | | |
| 178205 | 13q21.1 | S | M |
| (Q8N7V5) | | | |
| 178200 | 13q21.1 | S | P |
| (Q8N7V5) | | | |
| 177527 | 13q21.31 | E, S | P |
| (Q8N7F4) | | | |
| 185498 | 13q21.32 | E, S | P |
| 152192 | 13q31.1 | S | M |
| (POU4F1) | | | |
| 171650 | 13q31.1 | S | P |
| (PTA1A) | | | |
| 184052 | 13q31.1 | E | P |
| 165300 (Y918) | 13q31.2 | S | P |
| 139800 (ZIC5) | 13q32.3 | S | M |
| 102466 | 13q33.1 | E | M |
| (FGF14) | | | |
| 185950 (IRS2) | 13q34 | S | M |
| 153481 | 13q34 | S | M |
| (NM_018210) | | | |
| 126218 (F10) | 13q34 | S | M |
| 186009 | 13q34 | S | M |
| (ATP4B) | | | |
| 184497 | 13q34 | E, S | M |
| (FAM70B) | | | |
| 185989 | 13q34 | S | M |
| (RASA3) | | | |
| 176294 | 14q11.2 | E | P |
| (OR4N2) | | | |
| 136367 | 14q11.2 | S | M |
| (ZFHX2) | | | |
| 176165 | 14q12 | E, S | P |
| (FOXG1C) | | | |
| 136352 (TITF1) | 14q13.3 | S | M |
| 186215 | 14q13.3 | S | P |
| (Q86SZ3) | | | |
| 136327 | 14q13.3 | S | P |
| (NKX2-8) | | | |
| 151338 | 14q13.3 | E | M |
| (M1POL1) | | | |
| 151748 (SAV1) | 14q22.1 | E | M |
| 073712 | 14q22.1 | E, S | P |
| (PLEKHC1) | | | |
| 125378 (BMP4) | 14q22.2 | S | M |
| 184302 (SIX6) | 14q23.1 | S | P |
| 177126 | 14q24.3 | S | P |
| (C14orf141) | | | |
| 183992 | 14q31.1 | E, S | M |
| 140093 | 14q32.13 | E | P |
| (SERP1NA10) | | | |
| 036530 | 14q32.2 | S | P |
| (CYP46A1) | | | |
| 140107 | 14q32.2 | E | M |
| (Q86U14) | | | |
| 185469 (RTL1) | 14q32.31 | E, S | M |
| 066735 | 14q32.33 | E | M |
| (KIF26A) | | | |
| 184601 | 14q32.33 | S | M |
| (Q8N912) | | | |
| 130235 | 14q32.33 | S | M |
| (NM_032714) | | | |
| 1849J6 (JAG2) | 14q32.33 | S | M |
| 184552 | 14q32.33 | S | M |
| (Q8NAF8) | | | |
| J82351 | 14q32.33 | S | M |
| (CR1P1) | | | |
| 177199 (IGHA2) | 14q32.33 | S | M |
| 177154 (IGHE) | 14q32.33 | S | P |
| 177145 | 14q32.33 | S | M |
| (IGHG1) | | | |
| 126309 (HV1A) | 14q32.33 | S | P |
| 126290 (HV2A) | 14q32.33 | E, S | P |
| 151802 | 15q13.1 | E, S | P |
| (Q9P168) | | | |
| 103832 | 15q13.2 | E | P |
| (060374) | | | |
| 134146 | 15q14 | S | P |
| (NM_080650) | | | |
| 179315 | 15q.14 | S | P |
| 184263 | 15q21.2 | S | P |
| 169856 | 15q21.3 | S | M |
| (ONECUT1) | | | |
| 069667 (RORA) | 15q22.2 | S | M |
| 138622 (HCN4) | 15q24.1 | S | M |
| 186690 | 15q24.3 | S | P |
| 140557 | 15q26.1 | S | P |
| (SIAT8B) | | | |
| 183643 | 15q26.1 | E | M |
| (C15orf32) | | | |
| 184254 | 15q26.3 | S | M |
| (ALDH1A3) | | | |
| 140479 | 15q26.3 | S | M |
| (PACE4) | | | |
| 103326 (SOLH) | 16p13.1 | S | M |
| 127585 | 16p13.3 | S | M |
| (NM_153350) | | | |
| 127586 | 16p13.3 | S | M |
| (CHTF18) | | | |
| 005513 (SOX8) | 16p13.3 | E, S | P |
| 172268 | 16p13.3 | E, S | P |
| (Q96S05) | | | |
| 172257 | 16p13.3 | S | P |
| (Q96S03) | | | |
| 184471 | 16p13.3 | S | M |
| 073761 | 16p13.3 | S | M |
| (CACNA1H) | | | |
| 140650 (PMM2) | 16p13.2 | S | P |
| 182375 | 16p11.2 | S | P |
| 185836 | 16p11.2 | S | P |
| 102924 | 16q12.1 | S | M |
| (CBLN1) | | | |
| 103449 (SALL1) | 16q12.1 | E, S | M |
| 183022 | 16q12.2 | S | M |
| 103005 | 16q13 | E, S | M |
| (C16orf57) | | | |
| 102890 | 16q22.1 | S | P |
| (ELM03) | | | |
| 102977 (ACD) | 16q22.1 | E, S | M |
| 103056 | 16q22.1 | S | M |
| (SMPD3) | | | |
| 103241 | 16q24.1 | E, S | M |
| (FOXF1) | | | |
| 179588 | 16q24.2 | S | M |
| (ZFPM1) | | | |
| 051523 (CYBA) | 16q24.2 | S | M |
| 183788 | 16q24.3 | E, S | M |
| (Q8N206) | | | |
| 183518 | 17p13.3 | E, S | M |
| 183688 | 17p13.3 | S | M |
| (NM_182705) | | | |
| 167874 | 17p13.1 | E, S | M |
| (TMEM88) | | | |
| 109061 (MYH1) | 17p13.1 | S | P |
| 108448 | 17p12 | E | M |
| (TRIM16) | | | |
| 160516 | 17p11.2 | S | M |
| (RPS28) | | | |
| 181977 (PYY2) | 17q11.2 | E, S | P |
| 184142 (TIAF1) | 17q11.2 | E | M |
| 108587 | 17q11.2 | S | P |
| (GOSR1) | | | |
| 172716 | 17q12 | S | M |
| (NM_152270) | | | |
| 171532 | 17q12 | S | P |
| (NEUROD2) | | | |
| 173917 | 17q21.32 | E, S | M |
| (HOXB2) | | | |
| 120093 | 17q21.32 | E, S | M |
| (HOXB3) | | | |
| 182742 | 17q21.32 | S | M |
| (HOXB4) | | | |
| 108511 | 17q21.32 | S | M |
| (HOXB6) | | | |
| 120068 | 17q21.32 | S | M |
| (HOXB8) | | | |
| 141378 (YCE7) | 17q23.2 | E, S | M |
| 121068 (TBX2) | 17q23.2 | S | M |
| 187011 | 17q23.2 | E | M |
| (C17orf82) | | | |
| 136492 | 17q23.2 | S | P |
| (BR1P1) | | | |
| 125398 (SOX9) | 17q24.3 | S | P |
| 161547 | 17q25.1 | E | M |
| (SFRS2) | | | |
| 16728J | 17q25.3 | S | P |
| 141570 (CBX8) | 17q25.3 | S | M |
| 141582 (CBX4) | 17q25.3 | S | M |
| 175901 | 17q25.3 | S | M |
| (Q8NBT7) | | | |
| 181428 | 17q25.3 | E, S | P |
| (Q8N8L1) | | | |
| 181409 (AATK) | 17q25.3 | S | M |
| 187207 | 17q25.3 | S | M |
| 186765 | 17q25.3 | S | P |
| (FSCN2) | | | |
| 184703 (SIRT7) | 17q25.3 | S | M |
| 184715 | 17q25.3 | S | M |
| (NM_032711) | | | |
| 169750 (RAC3) | 17q25.3 | S | P |
| 169727 (GPS1) | 17q25.3 | S | M |
| 154655 | 18p11.31 | S | P |
| (NM_173464) | | | |
| 067900 | 18q11.1 | S | M |
| (ROCK1) | | | |
| 141448 | 18q11.2 | E | M |
| (GATA6) | | | |
| 141441 | 18q12.1 | E, S | P |
| (FAM59A) | | | |
| J01746 (NOL4) | 18q12.1 | S | M |
| 101489 | 18q12.2 | E, S | M |
| (BRUNOL4) | | | |
| 152217 | 18q12.3 | E | P |
| (SETBPJ) | | | |
| 183677 (ELA2) | 18q21.1 | S | M |
| 141644 (MBD1) | 18q21.1 | S | M |
| 041353 | 18q21.2 | E | P |
| (RAB27B) | | | |
| 141668 | 18q22.3 | S | P |
| (NM_182511) | | | |
| 141665 | 18q22.3 | S | P |
| (NM_152676) | | | |
| 101544 | 18q23 | S | M |
| (NM_104913) | | | |
| 178184 | 18q23 | S | P |
| (PARD6G) | | | |
| 141934 | 19p13.3 | E, S | M |
| (PPAP2C) | | | |
| 1J8050 | 19p13.3 | S | M |
| (NM_017914) | | | |
| 180866 | 19p13.2 | E, S | P |
| (Q8NB05) | | | |
| 105655 | 19p13.11 | S | M |
| (NM_016368) | | | |
| 172684 | 19p13.11 | E, S | P |
| (Q8NE65) | | | |
| 172666 | 19p13.11 | E, S | P |
| 187135 | 19q12 | S | M |
| 121297 (TSH3) | 19q12 | E, S | P |
| 130876 | 19q13.1 | 1S | M |
| (SLC7A10) | | | |
| 124302 | 19q13.11 | E, S | M |
| (CHST8) | | | |
| 105698 (USF2) | 19q13.12 | S | M |
| 126266 | 19q13.12 | S | M |
| (GPR40) | | | |
| 105663 (TRX2) | 19q13.12 | E | M |
| 180458 | 19q13.13 | E, S | P |
| (Q8N3U1) | | | |
| 105737 (GRIK5) | 19q13.2 | S | M |
| 159904 | 19q13.31 | E, S | P |
| (ZNF225) | | | |
| 167383 | 19q13.31 | E, S | M |
| (ZNF229) | | | |
| 176499 | 19q13.33 | E | M |
| (Q9Y4U5) | | | |
| 175856 | 19q13.41 | E | M |
| (Q8NB48) | | | |
| 186818 | 19q13.42 | E, S | M |
| (LILRB4) | | | |
| 105132 (ZN550) | 19q13.43 | E, S | M |
| 130724 | 19q13.43 | E, S | M |
| (CHMP2A) | | | |
| 099326 | 19q13.43 | E, S | M |
| (ZNF42) | | | |
| 175487 | 19q13.43 | S | P |
| (Q9BPX8) | | | |
| 178591 | 20p13 | S | M |
| (DEFB125) | | | |
| 088782 | 20p13 | S | P |
| (DEFB127) | | | |
| 125906 | 20p13 | E | P |
| (Q9H410) | | | |
| 125861 | 20p13 | S | P |
| (GFRA4) | | | |
| 101230 | 20p12.1 | E, S | P |
| (C20orf82) | | | |
| 172264 | 20p12.1 | S | M |
| (C20orf133) | | | |
| 125798 | 20p11.21 | S | M |
| (FOXA2) | | | |
| 125810 | 20p11.21 | S | M |
| (CIQR1) | | | |
| 125831 | 20p11.21 | S | M |
| (CSTJ1) | | | |
| 154930 | 20p11.21 | E | P |
| (ACAS2L) | | | |
| 183029 | 20q11.21 | S | P |
| (Q8NCY9) | | | |
| 026559 | 20q13.13 | S | P |
| (KCNG1) | | | |
| 124222 | 20q13.32 | E | P |
| (STX16) | | | |
| 179242 (CDH4) | 20q13.33 | S | M |
| 101180 (HRH3) | 20q13.33 | S | M |
| 130702 | 20q13.33 | S | M |
| (LAMA5) | | | |
| 174407 | 20q13.33 | S | M |
| (C20orf166) | | | |
| 101188 | 20q13.33 | S | M |
| (NTSR1) | | | |
| 101189 | 20q13.33 | E, S | M |
| (C20orf20) | | | |
| 060491 (OGFR) | 20q13.33 | S | M |
| 092758 | 20q13.33 | E, S | M |
| (COL9A3) | | | |
| 101204 | 20q13.33 | E | M |
| (CHRNA4) | | | |
| 075043 | 20q13.33 | S | M |
| (KCNQ2) | | | |
| 130589 (P285) | 20q13.33 | E | M |
| 125520 | 20q13.33 | S | M |
| (SLC2A4RG) | | | |
| 171700 | 20q13.33 | S | M |
| (RGS19) | | | |
| 171695 | 20q13.33 | S | P |
| (Q8TD35) | | | |
| 181872 | 20q13.33 | S | P |
| 175302 | 21q11.2 | S | P |
| (Q9NS19) | | | |
| 184856 | 21q21.1 | E | P |
| (C21orf74) | | | |
| 186930 | 21q22.11 | S | P |
| (KRTAP6-2) | | | |
| J85569 (OLIG2) | 21q22.11 | S | M |
| 159263 (SIM2) | 21q22.13 | E, S | P |
| 183067 | 21q22.2 | E | P |
| (Q9NS15) | | | |
| 141956 | 21q22.3 | S | M |
| (PRDM15) | | | |
| 014442 | 21q22.3 | E | M |
| (ADARB1) | | | |
| 182586 | 21q22.3 | E | P |
| (C21orf89) | | | |
| 186866 | 21q22.3 | S | P |
| (C21orf80) | | | |
| 187153 | 21q22.3 | S | M |
| 142156 | 21q22.3 | S | M |
| (COL6A1) | | | |
| 160294 | 21q22.3 | E | M |
| (MCM3AP) | | | |
| 160305 (D1P2) | 21q22.3 | S | M |
| 160307 (S100B) | 21q22.3 | E | P |
| 160310 | 21q22.3 | E | P |
| (HRMTIL1) | | | |
| 183628 | 22q11.21 | E, S | M |
| (DGCR6) | | | |
| 100075 | 22q11.21 | S | M |
| (SLC25A1) | | | |
| 183099 | 22q11.21 | E, S | M |
| 100208 (IGLC1) | 22q11.22 | S | P |
| 186746 | 22q11.22 | E | M |
| 178803 | 22q11.23 | S | P |
| (Q8NAW6) | | | |
| 100104 (SRR1) | 22q12.1 | E | M |
| 169184 (MN1) | 22q12.1 | S | P |
| 184390 | 22q12.2 | E, S | P |
| (Q61CM0) | | | |
| 166897 | 22q13.1 | S | P |
| (Q96PY3) | | | |
| 184687 | 22q13.31 | E, S | P |
| (Q8ND38) | | | |
| 075275 | 22q13.31 | E | M |
| (CELSR1) | | | |
| 182858 | 22q13.33 | S | M |
| (NM_024105) | | | |
| 128159 | 22q133.3 | S | M |
| (TUBGCP6) | | | |
| 185386 | 22q13.33 | S | M |
| (MAPK12) | | | |
| 100239 (K685) | 22q13.33 | S | P |
| 025770 | 22q13.33 | S | M |
| (NM_014551) | | | |
| 182786 | 22q13.33 | S | P |

Genes predicted to be imprinted by both the linear and REF kernel classifiers learned by Equbits are denoted by E, and those predicted by both the linear and RBF kernel classifiers learned by SMLR by S. Genes predicted to be imprinted by both programs are denoted by E,S and represent the ‘high-confidence’ set presented in Table 1 hereinabove. Genes predicted to be expressed from the maternal or paternal allele are denoted by M or P, respectively. To enhance legibility, the common prefix “ENSG00000” has been dropped from the Ensembl ID. Also listed are gene names and/or GENBANK® Accession Nos. where applicable.

TABLE 3
Chromosomal Bands with High Frequencies of Genes Proved or
Predicted with High Confidence to be Imprinted

| | Freq. | | |
|---|---|---|---|
| Band | (# known) | P | Novel Candidates |
| 11p15.5 | 10/82 (5) | <3 × 10⁻¹⁶ | PKP3, an oncogene involved in lung cancer |
| | | | (Furukawa et al., 2005), located distal to |
| | | | the IGF2/H19 cluster. |
| 1p36.32 | 5/24 (1) | <3 × 10⁻¹⁶ | PRDM16, whose ortholog was also predicted |
| | | | to be imprinted in mouse (Luedi et al., |
| | | | 2005), and is associated with leukemia (Du |
| | | | et al., 2005). |
| 7p15.2 | 6/26 (0) | <3 × 10⁻¹⁶ | Several loci are involved in development and |
| | | | are susceptible to epigenetic regulation. |
| 10q26.3 | 6/44 (0) | 9 × 10⁻¹⁶ | NKX6-2 (also predicted to be imprinted in the |
| | | | mouse; Luedi et al., 2005), is preferentially |
| | | | expressed in the brain (Lee et al., 2001). |
| | | | Along with five neighboring candidate |
| | | | genes, was predicted to show maternal |
| | | | expression. Near the marker D10S217 |
| | | | (maternally linked to male sexual |
| | | | orientation (Mustanski et al., 2005). A |
| | | | germline differentially methylated region |
| | | | was also found in this region (Strichman- |
| | | | Almashanu et al., 2002). |
| 14q32.31 | 2/5 (1) | 1 × 10⁻¹¹ | RTL1, imprinted in the mouse (Seitz et al., |
| | | | 2003) and sheep (Charlier et al., 2001). |
| 15q12 | 2/6 (2) | 7 × 10⁻¹⁰ | |
| 7q21.3 | 4/35 (4) | 2 × 10⁻⁸ | |

TABLE 4
Relevant Features for Prediction of Imprinting by Equbits Classifiers

| | | Mean (Standard deviation) | | | | |
|---|---|---|---|---|---|---|
| Feature | Weight | All Genes | | Imprinted | | P |
| downstream 10:100 SINE_Alu±² | −16.96 | 4.11 | (61.50) | 1.68 | (0.81) | 4.76 × 10⁻⁹ |
| downstream 10:100 AluS±² | 11.28 | 16.89 | (162.70) | 161.12 | (557.13) | 5.70 × 10⁻² |
| upstream 60:0 LTR_ERVL±² | 10.08 | 11.31 | (21.28) | 34.03 | (55.48) | 7.28 × 10⁻³ |
| upstream 9:8 Sp1¹ | −9.75 | 0.34 | (1.09) | 0.15 | (0.53) | 1.64 × 10⁻² |
| upstream 100:10 AluJ±² | 9.16 | 61.09 | (228.98) | 193.30 | (372.53) | 1.63 × 10⁻² |
| upstream 5:4 NFuE1¹ | 9.14 | 0.04 | (0.21) | 0.13 | (0.33) | 6.88 × 10⁻² |
| downstream 0:5 MIR3² | 8.89 | 0.33 | (0.99) | 0.45 | (1.34) | 2.91 × 10⁻¹ |
| upstream 100:downstream 100 CCACGTGG within | 8.86 | 0.13 | (0.33) | 0.30 | (0.46) | 1.31 × 10⁻² |
| THE1B/B-int elements³ | | | | | | |
| upstream 8:7 GTIIC¹ | 8.73 | 0.34 | (0.61) | 0.53 | (0.78) | 7.02 × 10⁻² |
| upstream 3:2 Sp1¹ | 8.59 | 0.36 | (1.13) | 0.93 | (1.40) | 8.41 × 10⁻³ |
| upstream 5:0 LTR_ERV1¹ | 8.58 | 0.36 | (1.07) | 0.58 | (2.11) | 2.68 × 10⁻¹ |
| downstream 5:10 L1ME±¹ | −8.57 | 0.01 | (0.99) | −0.28 | (1.18) | 6.79 × 10⁻² |
| intron LTR_ERV1±² | 8.39 | 173.80 | (743.12) | 617.58 | (1711.93) | 5.68 × 10⁻² |
| upstream 100:downstream 100 CCACGTGG within | 8.27 | 0.15 | (0.43) | 0.43 | (0.78) | 1.70 × 10⁻² |
| THE1B/B-int elements¹ | | | | | | |
| downstream 10:100 L1M4² | 8.22 | 0.70 | (1.11) | 1.45 | (1.77) | 5.85 × 10⁻³ |
| intron CpGi² | 8.17 | 44.47 | (86.92) | 102.33 | (186.25) | 2.98 × 10⁻² |
| upstream 10:9 Sp1¹ | −8.15 | 0.34 | (1.11) | 0.30 | (0.61) | 3.46 × 10⁻¹ |
| downstream 10:100 L1P² | −8.14 | 0.31 | (1.06) | 0.14 | (0.58) | 3.61 × 10⁻² |
| downstream 10:100 SINE_MIR±¹ | −8.13 | 0.11 | (2.25) | −0.41 | (2.31) | 8.33 × 10⁻² |
| upstream 50:0 L2±¹ | 7.97 | 0.29 | (2.90) | 1.05 | (4.42) | 1.47 × 10⁻¹ |
| upstream 100:10 L1ME±¹ | −7.94 | 0.29 | (4.79) | −1.72 | (3.94) | 1.42 × 10⁻³ |
| upstream 2:1 PEA2¹ | 7.91 | 0.02 | (0.15) | 0.05 | (0.22) | 2.18 × 10⁻¹ |
| upstream 5:4 AP1¹ × downstream 0:100 MLT1C phase | −7.89 | 0.77 | (2.32) | 0.03 | (0.16) | 0 |
| change² | | | | | | |
| downstream 0:5 MIR3¹ | 7.84 | 0.14 | (0.41) | 0.18 | (0.50) | 3.43 × 10⁻¹ |
| downstream 5:10 L1PB¹ | 7.79 | 0.04 | (0.29) | 0.13 | (0.56) | 1.75 × 10⁻¹ |
| upstream 100:10 DNA_Tip100¹ × upstream 6:5 GTIIC³ | −7.77 | 0.18 | (0.72) | 0.00 | (0.00) | 0 |
| downstream 0:5 MIR3±¹ | 7.65 | 0.01 | (0.41) | 0.10 | (0.44) | 1.01 × 10⁻¹ |
| upstream 4:3 PEA1¹ | 7.6 | 0.10 | (0.32) | 0.13 | (0.40) | 3.50 × 10⁻¹ |
| upstream 100:10 AluJ±¹ | 7.57 | 0.27 | (2.37) | 1.17 | (2.83) | 2.73 × 10⁻² |
| intron LTR_ERV1±¹ | −7.53 | −0.49 | (2.02) | −1.75 | (4.90) | 5.78 × 10⁻² |
| downstream 5:10 L1MC±² | 7.5 | 71.64 | (271.78) | 129.05 | (386.82) | 1.80 × 10⁻¹ |
| upstream 100:10 L1MA±¹ | 7.36 | 0.07 | (2.70) | 1.13 | (3.64) | 3.81 × 10⁻² |
| upstream 6:5 SIF³ × upstream 2:0 BPVE2³ | −7.31 | 0.13 | (0.33) | 0.00 | (0.00) | 0 |
| upstream 9:8 CEBP¹ | 7.29 | 0.04 | (0.20) | 0.10 | (0.38) | 1.64 × 10⁻¹ |
| downstream 40:100 LTR_ERV1±¹ | 7.26 | 0.59 | (2.87) | 1.95 | (6.14) | 8.60 × 10⁻² |
| exon 0.225:0.41 nucleosome potential² | −7.22 | 0.67 | (0.92) | −0.10 | (1.06) | 2.65 × 10⁻⁵ |
| upstream 3:2 Sp1³ | 7.17 | 0.21 | (0.41) | 0.43 | (0.50) | 5.46 × 10⁻³ |
| downstream 5:10 Alu² | 7.08 | 0.01 | (0.06) | 0.01 | (0.09) | 3.05 × 10⁻¹ |
| upstream 100:10 MIR3±¹ | 7.07 | 0.14 | (1.84) | 0.51 | (1.74) | 9.53 × 10⁻² |
| upstream 3:2 BPVE2¹ × upstream 1:0 Pit1³ | −7.06 | 0.14 | (0.44) | 0.00 | (0.00) | 0 |
| upstream 30:20 CpGi^{2,10} | 7.02 | 0.13 | (0.28) | 0.17 | (0.35) | 2.47 × 10⁻¹ |
| upstream 100:10 LTR_ERV1±² | −6.98 | 459.12 | (1253.59) | 183.25 | (433.61) | 1.56 × 10⁻⁴ |
| upstream 100:10 L1MC±¹ | 6.96 | 0.24 | (4.23) | 1.23 | (5.64) | 1.40 × 10⁻¹ |
| upstream 8:7 EFC¹ | 6.87 | 0.00 | (0.04) | 0.03 | (0.16) | 1.82 × 10⁻¹ |
| upstream 8:7 GT2B¹ × upstream 9:8 Sp1³ | −6.86 | 0.11 | (0.44) | 0.00 | (0.00) | 0 |
| upstream 9:8 Sp1³ × upstream 8:7 GT2B³ | −6.82 | 0.08 | (0.27) | 0.00 | (0.00) | 0 |
| downstream 5:10 LINE_L2±² | 6.8 | 106.50 | (243.27) | 116.37 | (272.10) | 4.11 × 10⁻¹ |
| upstream distance to closest recomb. hotspot | −6.79 | 315.02 | (1369.12) | 122.40 | (94.52) | 0 |
| upstream 100:10 L1M4±² × upstream 1:0 MLTF³ | −6.78 | 229.05 | (605.18) | 25.45 | (91.09) | 0 |
| upstream 8:7 SIF³ × upstream 5:0 ETFA³ | −6.76 | 0.06 | (0.24) | 0.00 | (0.00) | 0 |
| upstream 5:0 LINE_CR1±² | 6.75 | 12.41 | (69.05) | 38.48 | (121.07) | 9.33 × 10⁻² |
| intron L1M2¹ | −6.73 | 0.05 | (0.37) | 0.00 | (0.00) | 0 |
| upstream 5:4 AP1³ × downstream 0:100 MLT1C phase | −6.71 | 0.34 | (0.85) | 0.03 | (0.16) | 5.55 × 10⁻¹⁶ |
| change² | | | | | | |
| upstream 9:8 MLTF³ | 6.62 | 0.57 | (0.50) | 0.70 | (0.46) | 4.03 × 10⁻² |
| upstream 5:4 AP1¹ × downstream 0:100 MLT1C phase | −6.59 | 0.23 | (0.69) | 0.01 | (0.08) | 0 |
| change² | | | | | | |
| upstream 6:5 SIF¹ × upstream 2:0 BPVE2³ | −6.57 | 0.16 | (0.50) | 0.00 | (0.00) | 0 |
| upstream 0.83:0.61 nucleosome potential¹ | −6.55 | 182.33 | (154.05) | 102.79 | (169.84) | 2.87 × 10⁻³ |
| downstream 5:10 L1MC² | 6.54 | 0.78 | (2.85) | 1.29 | (3.87) | 2.06 × 10⁻¹ |
| downstream 5:10 DNA_MER2_type±² | 6.52 | 46.30 | (230.76) | 190.05 | (602.01) | 7.20 × 10⁻² |
| downstream 5:10 CpGi¹ × upstream 10:9 NFuE5³ | −6.51 | 0.12 | (0.44) | 0.00 | (0.00) | 0 |
| upstream 7:6 NFuE5³ | 6.47 | 0.28 | (0.45) | 0.35 | (0.48) | 1.73 × 10⁻¹ |
| upstream 8:7 SiF¹ × upstream 8:7 BPVE2³ | −6.45 | 0.10 | (0.37) | 0.00 | (0.00) | 0 |
| upstream 8:7 ICP4¹ | −6.44 | 0.05 | (0.24) | 0.03 | (0.16) | 1.58 × 10⁻¹ |
| upstream 4:3 PEA2¹ | 6.43 | 0.02 | (0.13) | 0.10 | (0.30) | 4.92 × 10⁻² |
| upstream 100:10 DNA_Tip100¹ × upstream 3:2 Pit1³ | −6.42 | 0.29 | (0.89) | 0.00 | (0.00) | 0 |
| downstream 0:100 MLT1A0 phase change¹ | 6.37 | 0.40 | (0.86) | 0.83 | (1.03) | 7.45 × 10⁻³ |
| upstream 1:0 BPVE2¹ × upstream 10:9 NFuE5³ | −6.32 | 0.12 | (0.40) | 0.00 | (0.00) | 0 |
| upstream 9:8 MLTF¹ | 6.31 | 0.91 | (1.13) | 1.23 | (1.10) | 4.28 × 10⁻² |
| upstream 7:6 NFuE4¹ | 6.29 | 0.04 | (0.21) | 0.05 | (0.22) | 3.85 × 10⁻¹ |
| upstream 100:10 DNA_Tip100±² | −6.24 | 90.37 | (251.03) | 69.05 | (309.29) | 3.35 × 10⁻¹ |
| upstream 9:8 CEBP³ | 6.22 | 0.04 | (0.19) | 0.08 | (0.27) | 1.99 × 10⁻¹ |
| upstream 2:0 Oct1³ | 6.18 | 0.61 | (0.49) | 0.73 | (0.45) | 5.29 × 10⁻² |
| upstream 10:9 GT2B¹ × upstream 3:2 Pit1³ | −6.17 | 0.17 | (0.49) | 0.00 | (0.00) | 0 |
| downstream 5:10 AluY±² | 6.12 | 80.80 | (155.01) | 145.40 | (217.27) | 3.55 × 10⁻² |
| downstream 10:100 MIR±¹ | −6.11 | 0.12 | (2.35) | −0.23 | (2.46) | 1.84 × 10⁻¹ |
| upstream 2:0 CpGi¹ × upstream 9:8 E4F1³ | −6.08 | 0.07 | (0.26) | 0.00 | (0.00) | 0 |
| upstream 8:7 GTIIC³ | 6.07 | 0.28 | (0.45) | 0.38 | (0.49) | 1.05 × 10⁻¹ |
| downstream 10:100 FLAM±² × upstream 10:9 ATF¹ | −6.06 | 34.06 | (129.03) | 0.22 | (0.62) | 0 |
| upstream 40:30 CpGi^{1,10} | 6.05 | 0.36 | (0.85) | 0.45 | (1.30) | 3.28 × 10⁻¹ |
| downstream 5:10 DNA_MER2_type² | 6.03 | 0.50 | (2.39) | 1.90 | (6.02) | 7.69 × 10⁻² |
| upstream 6:5 ATF³ | 6.01 | 0.36 | (0.48) | 0.48 | (0.51) | 7.39 × 10⁻² |
| upstream 5:0 LINE_CR1² | 6 | 0.26 | (1.42) | 0.77 | (2.42) | 9.71 × 10⁻² |
| upstream 9:8 Sp1³ × upstream 1:0 ICSBP³ | −5.95 | 0.14 | (0.35) | 0.00 | (0.00) | 0 |
| upstream 7:6 NFkB³ | 5.92 | 0.08 | (0.27) | 0.15 | (0.36) | 1.14 × 10⁻¹ |
| upstream 100:10 HAL1±² × upstream 9:8 TFIID³ | −5.86 | 141.37 | (392.44) | 13.28 | (47.90) | 0 |
| upstream 2:0 MIR² × upstream 8:7 GT2B³ | −5.85 | 0.79 | (2.90) | 0.00 | (0.00) | 0 |
| downstream 10:100 DNA¹ × upstream 10:9 PU1³ | −5.83 | 0.23 | (0.66) | 0.00 | (0.00) | 0 |
| exon LINE_L2±¹ | 5.82 | 0.00 | (0.23) | 0.05 | (0.22) | 8.77 × 10⁻² |
| upstream 100:10 DNA_Tip100¹ × upstream 8:7 ICSBP³ | −5.79 | 0.53 | (1.14) | 0.08 | (0.27) | 1.61 × 10⁻¹³ |
| upstream 100:10 DNA_Tip100² × upstream 8:7 ICSBP³ | −5.78 | 0.09 | (0.27) | 0.01 | (0.04) | 0 |
| upstream 2:0 L1M2±¹ | 5.74 | 0.00 | (0.10) | 0.03 | (0.16) | 1.82 × 10⁻¹ |
| upstream 100:10 L1P¹ | −5.71 | 0.42 | (0.96) | 0.28 | (0.55) | 6.08 × 10⁻² |
| downstream 5:10 LTR_ERVK±¹ | −5.7 | 0.00 | (0.26) | −0.15 | (0.95) | 1.64 × 10⁻¹ |
| downstream 0:1 CpGi¹ | −5.68 | 0.04 | (0.19) | 0.03 | (0.16) | 2.86 × 10⁻¹ |
| upstream 100:10 HAL1¹ × upstream 8:7 GT2B¹ | −5.66 | 0.49 | (1.88) | 0.03 | (0.16) | 0 |
| downstream 10:100 MIR3±² × upstream 4:3 GATA1¹ | −5.65 | 48.09 | (147.61) | 1.72 | (9.34) | 0 |
| downstream 5:10 LTR_ERVK¹ | 5.64 | 0.03 | (0.27) | 0.15 | (0.95) | 2.23 × 10⁻¹ |
| downstream 10:100 L1MB² | 5.61 | 1.24 | (1.54) | 1.67 | (2.02) | 9.54 × 10⁻² |
| upstream 2:0 MIR¹ × upstream 8:7 GT2B³ | −5.58 | 0.12 | (0.45) | 0.00 | (0.00) | 0 |
| upstream 100:10 DNA±² | 5.57 | 45.51 | (150.10) | 99.38 | (312.29) | 1.44 × 10⁻¹ |
| downstream 10:100 LTR_ERVK² | 5.55 | 0.38 | (1.24) | 0.83 | (1.89) | 7.35 × 10⁻² |
| upstream 9:8 Sp1³ × upstream 10:0 CpGi^{1,11} | −5.53 | 0.66 | (1.70) | 0.08 | (0.27) | 0 |
| upstream 2:1 SIF³ | 5.49 | 0.24 | (0.43) | 0.28 | (0.45) | 3.22 × 10⁻¹ |
| downstream 0:100 LTR phase change^{2,99} | 5.48 | 0.29 | (0.28) | 0.40 | (0.27) | 1.09 × 10⁻² |
| upstream 100:10 DNA_Tip100¹ × upstream 8:7 ICSBP¹ | −5.47 | 1.00 | (2.50) | 0.10 | (0.38) | 0 |
| upstream 5:0 CpGi¹ × upstream 9:8 E4F1³ | −5.46 | 0.09 | (0.32) | 0.00 | (0.00) | 0 |
| upstream 10:5 L1MC² | −5.45 | 0.77 | (2.82) | 0.57 | (2.26) | 2.83 × 10⁻¹ |
| upstream 4:3 E2F¹ | −5.43 | 0.01 | (0.11) | 0.00 | (0.00) | 0 |
| upstream 2:0 SINE_MIR² × upstream 8:7 GT2B³ | −5.42 | 0.93 | (3.19) | 0.00 | (0.00) | 0 |
| upstream 1:0 CP1³ | −5.41 | 0.07 | (0.25) | 0.03 | (0.16) | 5.73 × 10⁻² |
| downstream 0:5 DNA_Tip100² | −5.4 | 0.10 | (1.02) | 0.00 | (0.00) | 0 |
| downstream 10:100 L1MC¹ × downstream 0:100 MLT1C | −5.39 | 1.96 | (5.89) | 0.13 | (0.40) | 0 |
| phase change² | | | | | | |
| downstream 5:10 CpGi2¹ | 5.37 | 2.26 | (2.27) | 2.93 | (2.55) | 5.61 × 10⁻² |
| intron CpGi³ | 5.36 | 0.08 | (0.27) | 0.28 | (0.45) | 5.48 × 10⁻³ |
| intron CpGi¹ | 5.35 | 0.47 | (1.10) | 1.05 | (1.52) | 1.10 × 10⁻² |
| downstream 0:2 AluY¹ × upstream 10:9 MLTF³ | −5.33 | 0.07 | (0.30) | 0.00 | (0.00) | 0 |
| upstream 9:8 PEA2¹ | −5.32 | 0.02 | (0.14) | 0.03 | (0.16) | 4.06 × 10⁻¹ |
| upstream 350:350 downstream recomb¹ | 5.28 | 4.84 | (2.75) | 6.15 | (2.18) | 2.96 × 10⁻⁴ |
| downstream 5:10 DNA_MER1_type¹ × upstream 3:2 Pit1³ | −5.27 | 0.15 | (0.53) | 0.00 | (0.00) | 0 |
| upstream 10:5 LTR_MaLR±² × upstream 6:5 APF³ | −5.26 | 94.23 | (233.79) | 3.92 | (23.86) | 0 |
| upstream 9:8 ATF³ × upstream 5:4 NFuE5³ | −5.25 | 0.11 | (0.31) | 0.00 | (0.00) | 0 |
| upstream 100:10 L1PB² | 5.24 | 0.49 | (1.48) | 0.69 | (1.89) | 2.64 × 10⁻¹ |
| downstream 5:10 L1MD±¹ | 5.23 | 0.00 | (0.47) | 0.00 | (0.45) | 5.00 × 10⁻¹ |
| downstream 0:2 MIR² | 5.21 | 1.73 | (3.99) | 1.80 | (4.79) | 4.63 × 10⁻¹ |
| upstream 5:0 PEA2³ | 5.2 | 0.12 | (0.32) | 0.25 | (0.44) | 3.12 × 10⁻² |
| upstream 9:8 Sp1¹ × upstream 1:0 ICSBP³ | −5.19 | 0.24 | (0.93) | 0.00 | (0.00) | 0 |
| downstream 5:10 LINE_L2² | 5.18 | 1.52 | (3.00) | 1.65 | (3.31) | 3.99 × 10⁻¹ |
| upstream 100:10 L1MB±² × upstream 5:4 ATF³ | −5.17 | 188.41 | (642.54) | 6.87 | (40.74) | 0 |
| intron DNA±¹ | −5.16 | 0.00 | (0.50) | −0.15 | (0.58) | 5.62 × 10⁻² |
| intron MIR3² × upstream 4:3 TFIID¹ | −5.15 | 16.59 | (66.36) | 1.43 | (6.31) | 0 |
| downstream 10:100 MIR3±² × upstream 4:3 GATA1³ | −5.12 | 33.26 | (88.69) | 1.62 | (9.32) | 0 |
| upstream 2:0 LINE_L2¹ × upstream 5:4 GATA1³ | −5.11 | 0.14 | (0.48) | 0.00 | (0.00) | 0 |
| upstream 9:8 NFkB¹ | 5.08 | 0.08 | (0.29) | 0.13 | (0.40) | 2.45 × 10⁻¹ |
| upstream 10:9 NFuE5³ × upstream 1:0 BPVE2³ | −5.07 | 0.10 | (0.30) | 0.00 | (0.00) | 0 |
| downstream 10:100 L1M4±¹ | 5.06 | 0.05 | (2.87) | 0.79 | (3.97) | 1.26 × 10⁻¹ |
| I⁵ × downstream 10:100 DNA_Mariner¹ | −5.05 | 0.17 | (0.57) | 0.00 | (0.00) | 0 |
| upstream 2:0 LINE_L2² | 5.04 | 2.88 | (7.56) | 3.35 | (10.80) | 3.93 × 10⁻¹ |
| downstream 5:10 CpGi¹ × upstream 10:9 NFuE5¹ | −5.03 | 0.15 | (0.63) | 0.00 | (0.00) | 0 |
| downstream 10:100 FLAM±² × upstream 10:9 ATF³ | −5.02 | 25.49 | (84.33) | 0.22 | (0.62) | 0 |
| upstream 5:0 LINE_CR1±¹ | −5.01 | 0.01 | (0.36) | −0.10 | (0.50) | 9.50 × 10⁻² |
| upstream 7:6 NFkB¹ | 4.98 | 0.08 | (0.29) | 0.15 | (0.36) | 1.27 × 10⁻¹ |
| upstream 5:0 L1M2±¹ | 4.97 | 0.00 | (0.16) | 0.03 | (0.16) | 1.94 × 10⁻¹ |
| downstream 10:100 LTR_ERV1² | −4.96 | 2.65 | (3.61) | 2.50 | (3.07) | 3.82 × 10⁻¹ |
| downstream 10:100 DNA_Mariner¹ × upstream 3:2 | −4.95 | 0.23 | (0.64) | 0.00 | (0.00) | 0 |
| MLTF³ | | | | | | |
| downstream 50:90 LTR_ERVL±¹ | 4.94 | 0.43 | (1.55) | 1.19 | (2.30) | 2.34 × 10⁻² |
| upstream 90:20 MIR±¹ | 4.93 | 0.21 | (2.27) | 0.85 | (2.69) | 7.48 × 10⁻² |
| upstream 5:0 LINE_CR1¹ | 4.92 | 0.07 | (0.36) | 0.20 | (0.46) | 5.03 × 10⁻² |
| upstream 6:5 PEA1³ | 4.91 | 0.10 | (0.29) | 0.18 | (0.38) | 1.01 × 10⁻¹ |
| upstream 2:1 Oct1³ | 4.9 | 0.42 | (0.49) | 0.50 | (0.51) | 1.62 × 10⁻¹ |
| upstream 8:7 E4F1¹ | 4.89 | 0.20 | (0.49) | 0.45 | (1.45) | 1.49 × 10⁻¹ |
| downstream 10:100 DNA² | −4.88 | 0.06 | (0.17) | 0.02 | (0.05) | 2.98 × 10⁻⁶ |
| downstream 10:100 L1MC¹ × downstream 0:100 MLT1C | −4.86 | 0.59 | (1.67) | 0.06 | (0.20) | 0 |
| phase change² | | | | | | |
| downstream 10:100 DNA±¹ | 4.85 | 0.02 | (0.80) | 0.10 | (0.50) | 1.56 × 10⁻¹ |
| upstream 4:3 E4F1³ | 4.84 | 0.18 | (0.38) | 0.20 | (0.41) | 3.77 × 10⁻¹ |
| upstream 10:5 LTR_ERV1±¹ | 4.83 | 0.01 | (1.05) | 0.13 | (1.02) | 2.47 × 10⁻¹ |
| upstream 2:0 LINE_L2±¹ | 4.82 | 0.03 | (0.68) | 0.23 | (0.58) | 2.01 × 10⁻² |
| downstream 5:10 MIR¹ | −4.81 | 0.77 | (1.14) | 0.40 | (0.50) | 2.14 × 10⁻⁵ |
| upstream 3:2 NFuE3¹ | 4.8 | 0.08 | (0.29) | 0.15 | (0.43) | 1.49 × 10⁻¹ |
| downstream 0:2 SINE_MIR² | 4.78 | 2.06 | (4.34) | 2.02 | (4.90) | 4.80 × 10⁻¹ |
| exon DNA_MER2_type² | 4.76 | 0.18 | (2.98) | 2.21 | (10.17) | 1.09 × 10⁻¹ |
| upstream 1:0 NFkB³ | 4.75 | 0.11 | (0.31) | 0.08 | (0.27) | 2.33 × 10⁻¹ |
| I⁵ × upstream 2:1 BPVE2³ | −4.74 | 0.16 | (0.37) | 0.00 | (0.00) | 0 |
| upstream 2:0 DNA_MER1_type¹ × upstream 6:5 AP1³ | −4.73 | 0.11 | (0.40) | 0.00 | (0.00) | 0 |
| upstream 2:0 LTR_ERV1±² | 4.72 | 34.74 | (150.24) | 60.40 | (234.23) | 2.49 × 10⁻¹ |
| upstream 5:4 SIF³ × upstream 4:3 PU1³ | −4.71 | 0.12 | (0.32) | 0.00 | (0.00) | 0 |
| downstream 5:10 DNA_MER2_type±¹ | −4.7 | 0.00 | (0.56) | −0.18 | (0.78) | 8.61 × 10⁻² |
| downstream 5:10 L1PA² | −4.68 | 2.70 | (10.90) | 2.43 | (10.47) | 4.37 × 10⁻¹ |
| upstream 9:8 SIF³ × m3_m11 | −4.67 | 0.09 | (0.29) | 0.00 | (0.00) | 0 |
| upstream 2:0 CpGi² | 4.66 | 152.21 | (161.47) | 222.15 | (212.49) | 2.33 × 10⁻² |
| downstream 10:100 DNA_MER1_type² × upstream 10:5 | −4.63 | 128.89 | (371.35) | 6.60 | (32.09) | 0 |
| LTR_MaLR±² | | | | | | |
| downstream 5:10 L1PA±² | −4.62 | 231.39 | (1001.15) | 179.41 | (978.02) | 3.71 × 10⁻¹ |
| upstream 100:10 HAL1¹ × upstream 8:7 GT2B³ | −4.61 | 0.37 | (1.26) | 0.03 | (0.16) | 0 |
| downstream 0:5 LTR_ERV1¹ | −4.59 | 0.28 | (0.92) | 0.10 | (0.38) | 3.00 × 10⁻³ |
| downstream 10:100 Other² | −4.57 | 0.19 | (0.60) | 0.06 | (0.27) | 1.37 × 10⁻³ |
| upstream 6:5 NF1³ | 4.56 | 0.12 | (0.33) | 0.18 | (0.38) | 2.00 × 10⁻¹ |
| downstream 0:1 CpGi² | −4.55 | 47.19 | (117.94) | 54.10 | (122.05) | 3.63 × 10⁻¹ |
| downstream 10:100 FLAM±² × upstream 100:10 MIR3±² | −4.53 | 5099.30 | (18824.72) | 54.26 | (133.79) | 0 |
| upstream 2:1 MLTF¹ | 4.52 | 0.96 | (1.06) | 1.10 | (1.22) | 2.34 × 10⁻¹ |
| upstream 100:10 L1M1±¹ | −4.51 | 0.04 | (0.87) | −0.23 | (0.97) | 4.79 × 10⁻² |
| upstream 5:0 LTR_MaLR±² | −4.5 | 87.69 | (215.92) | 64.15 | (132.75) | 1.38 × 10⁻¹ |
| upstream 10:5 LTR_MaLR±² × upstream 100:10 | −4.49 | 315.39 | (889.70) | 25.95 | (109.14) | 0 |
| LINE_L2² | | | | | | |
| downstream 10:100 L1MB¹ | 4.48 | 3.62 | (3.92) | 4.08 | (3.86) | 2.31 × 10⁻¹ |
| downstream 10:100 L1PB±¹ | −4.47 | 0.01 | (1.37) | 0.03 | (1.05) | 4.63 × 10⁻¹ |
| upstream 80:70 CpGi^{1,10} | −4.46 | 0.34 | (0.81) | 0.23 | (0.62) | 1.21 × 10⁻¹ |
| upstream 10:0 ETFA¹ × upstream 10:9 E4F1³ | −4.45 | 0.14 | (0.48) | 0.00 | (0.00) | 0 |
| upstream 5:0 LINE_L2±¹ | 4.44 | 0.05 | (1.24) | 0.30 | (1.32) | 1.23 × 10⁻¹ |
| upstream 3:2 APF¹ × downstream 0:100 MLT1C phase | −4.43 | 1.01 | (2.96) | 0.05 | (0.22) | 0 |
| change² | | | | | | |
| upstream 10:5 LTR_MaLR² × upstream 10:9 COUP³ | −4.42 | 1.26 | (2.92) | 0.12 | (0.57) | 7.77 × 10⁻¹⁶ |
| upstream 3:2 Oct1³ × downstream 0:100 MLT1C phase | −4.41 | 0.05 | (0.18) | 0.00 | (0.00) | 0 |
| change² | | | | | | |
| downstream 0:5 FRAM±¹ | −4.4 | 0.00 | (0.23) | −0.05 | (0.22) | 8.06 × 10⁻² |
| downstream 10:100 CpGi³ × upstream 100:10 L1MB±² | −4.39 | 286.48 | (761.12) | 22.01 | (95.07) | 0 |
| downstream 10:100 DNA_Mariner¹ × upstream 10:0 | −4.38 | 0.17 | (0.55) | 0.00 | (0.00) | 0 |
| ETFA³ | | | | | | |
| downstream 35:68 L2±¹ | 4.37 | 0.52 | (2.58) | 0.98 | (2.07) | 8.56 × 10⁻² |
| upstream 7:6 E2F¹ | 4.36 | 0.01 | (0.10) | 0.05 | (0.22) | 1.35 × 10⁻¹ |
| upstream 5:4 NFuE5¹ × upstream 9:8 ATF³ | −4.35 | 0.13 | (0.43) | 0.00 | (0.00) | 0 |
| upstream 10:0 NFkB¹ | 4.34 | 0.85 | (0.98) | 1.08 | (1.02) | 9.27 × 10⁻² |
| exon L1² | 4.33 | 0.03 | (1.56) | 0.17 | (1.09) | 2.13 × 10⁻¹ |
| upstream 10:9 E4F1³ × upstream 3:2 MLTF³ | −4.32 | 0.10 | (0.30) | 0.00 | (0.00) | 0 |
| upstream 6:5 MLTF³ | 4.31 | 0.57 | (0.50) | 0.63 | (0.49) | 2.44 × 10⁻¹ |
| upstream 5:0 CpGi¹ × upstream 9:8 E4F1¹ | −4.3 | 0.11 | (0.43) | 0.00 | (0.00) | 0 |
| upstream 8:7 BPVE2³ × upstream 8:7 SIF³ | −4.29 | 0.09 | (0.28) | 0.00 | (0.00) | 0 |
| upstream 8:7 MLTF¹ | 4.28 | 0.90 | (1.08) | 1.20 | (1.86) | 1.60 × 10⁻¹ |
| downstream 10:100 HAL1±¹ | −4.27 | −0.03 | (1.92) | −0.44 | (2.23) | 1.29 × 10⁻¹ |
| downstream 5:10 MIR² | −4.26 | 1.02 | (1.55) | 0.58 | (0.76) | 4.61 × 10⁻⁴ |
| upstream 6:5 ATF¹ | 4.25 | 0.49 | (0.78) | 0.63 | (0.81) | 1.43 × 10⁻¹ |
| upstream 2:0 LINE_L2¹ × upstream 5:4 GATA1¹ | −4.24 | 0.20 | (0.75) | 0.00 | (0.00) | 0 |
| upstream 100:10 DNA_Mariner¹ | −4.23 | 0.41 | (0.83) | 0.23 | (0.58) | 2.43 × 10⁻² |
| upstream 2:0 DNA_MER1_type¹ × upstream 10:9 AP1³ | −4.22 | 0.11 | (0.40) | 0.00 | (0.00) | 0 |
| upstream 100:60 LTR_ERVL±¹ | −4.21 | 0.41 | (1.56) | −0.11 | (3.58) | 1.84 × 10⁻¹ |
| downstream 10:100 L1M1² | −4.2 | 0.12 | (0.64) | 0.18 | (0.66) | 2.84 × 10⁻¹ |
| upstream 5:0 LINE_L2±² | 4.19 | 112.34 | (241.50) | 164.27 | (305.43) | 1.48 × 10⁻¹ |
| intron DNA_Tip100¹ | −4.18 | 0.19 | (0.76) | 0.10 | (0.38) | 7.26 × 10⁻² |
| downstream 0:5 LTR_ERV1² | −4.17 | 2.52 | (10.32) | 0.41 | (1.91) | 1.65 × 10⁻⁸ |
| upstream 9:8 GATA1³ × downstream 0:100 MLT1C | −4.15 | 0.06 | (0.18) | 0.00 | (0.00) | 0 |
| phase change² | | | | | | |
| upstream 6:5 APF³ × upstream 5:4 E4F1³ | −4.14 | 0.15 | (0.35) | 0.00 | (0.00) | 0 |
| upstream 10:9 NFuE5¹ × upstream 10:0 NFuE3³ | −4.13 | 0.17 | (0.48) | 0.00 | (0.00) | 0 |
| upstream 10:9 E4F1¹ × upstream 9:8 PU1³ | −4.12 | 0.12 | (0.38) | 0.00 | (0.00) | 0 |
| downstream 5:10 L1PB² | 4.11 | 0.35 | (3.49) | 1.46 | (6.49) | 1.45 × 10⁻¹ |
| upstream 10:5 SINE_MIR±² × upstream 7:6 BPVE2³ | −4.1 | 23.93 | (74.12) | 0.02 | (0.14) | 0 |
| intron DNA_Tip100±¹ | −4.09 | 0.00 | (0.59) | 0.00 | (0.39) | 4.73 × 10⁻¹ |
| upstream 10:5 LTR_ERVL±¹ | 4.08 | 0.00 | (0.66) | 0.18 | (0.75) | 7.32 × 10⁻² |
| upstream 100:10 HAL1±² × upstream 9:8 TFIID¹ | −4.07 | 264.58 | (860.20) | 17.11 | (63.06) | 0 |
| upstream 10:9 E4F1¹ × upstream 10:0 ETFA³ | −4.06 | 0.11 | (0.38) | 0.00 | (0.00) | 0 |
| upstream 2:0 DNA_MER1_type±² | −4.05 | 19.25 | (74.01) | 2.20 | (13.91) | 1.34 × 10⁻⁹ |
| upstream 10:9 NFuE5¹ × upstream 1:0 BPVE2³ | −4.04 | 0.12 | (0.42) | 0.00 | (0.00) | 0 |
| upstream 100:10 L1MB¹ | 4.03 | 3.70 | (3.98) | 4.38 | (4.80) | 1.93 × 10⁻¹ |
| upstream 100:10 LTR_ERV1¹ × upstream 10:0 NFuE4³ | −4.02 | 2.12 | (5.53) | 0.18 | (0.50) | 0 |
| upstream 7:6 CP1¹ | 4.01 | 0.05 | (0.26) | 0.08 | (0.27) | 2.84 × 10⁻¹ |
| upstream 100:10 L1MD±¹ | −4 | 0.04 | (2.40) | −0.06 | (2.73) | 4.09 × 10⁻¹ |
| upstream 2:1 AP2¹ | 3.99 | 0.42 | (0.82) | 0.90 | (1.46) | 2.38 × 10⁻² |
| downstream 10:100 DNA_Tc2±¹ | −3.98 | −0.01 | (0.63) | −0.03 | (0.66) | 4.34 × 10⁻¹ |
| downstream 10:100 DNA_MER2_type±¹ | 3.97 | 0.11 | (2.47) | 0.58 | (1.90) | 6.53 × 10⁻² |
| upstream 9:8 ATF¹ × upstream 5:4 NFuE5¹ | −3.96 | 0.19 | (0.74) | 0.00 | (0.00) | 0 |
| upstream 6:5 GT2B¹ | 3.95 | 0.44 | (0.77) | 0.70 | (0.94) | 4.81 × 10⁻² |
| downstream 10:100 DNA_Mariner¹ × upstream 1:0 ATF¹ | −3.94 | 0.35 | (1.23) | 0.00 | (0.00) | 0 |
| upstream 100:10 DNA² | 3.93 | 0.06 | (0.17) | 0.10 | (0.31) | 2.08 × 10⁻¹ |
| upstream 10:9 E4F1³ × upstream 3:2 GATA1³ | −3.92 | 0.08 | (0.27) | 0.00 | (0.00) | 0 |
| upstream 10:5 LTR_MaLR±² × upstream 3:2 AP1³ | −3.9 | 91.79 | (229.88) | 0.15 | (0.65) | 0 |
| upstream 4:3 NFuE5³ × upstream 4:3 Pit1³ | −3.89 | 0.10 | (0.30) | 0.00 | (0.00) | 0 |
| upstream 10:9 GT2B¹ × upstream 1:0 MLTF¹ | −3.88 | 0.58 | (1.54) | 0.08 | (0.27) | 5.88 × 10⁻¹⁵ |
| downstream 10:100 DNA_Mariner¹ × upstream 1:0 TFIID³ | −3.87 | 0.21 | (0.62) | 0.00 | (0.00) | 0 |
| downstream 0:2 LTR_ERVL¹ | −3.86 | 0.06 | (0.37) | 0.00 | (0.00) | 0 |
| exon LINE_L2¹ | 3.85 | 0.04 | (0.24) | 0.05 | (0.22) | 3.73 × 10⁻¹ |
| upstream 1:0 NFuE3¹ | −3.84 | 0.05 | (0.24) | 0.03 | (0.16) | 1.31 × 10⁻¹ |
| upstream 10:9 GTIIC³ × upstream 4:3 BPVE2³ | −3.83 | 0.10 | (0.30) | 0.00 | (0.00) | 0 |
| upstream 7:6 COUP³ × upstream 2:1 E4TF1³ | −3.82 | 0.06 | (0.25) | 0.00 | (0.00) | 0 |
| upstream 10:5 DNA_MER2_type±² | −3.81 | 32.24 | (145.71) | 13.18 | (70.83) | 5.09 × 10⁻² |
| upstream 5:4 GATA1³ × downstream 0:100 MLT1C | −3.8 | 0.19 | (0.65) | 0.00 | (0.00) | 0 |
| phase change² | | | | | | |
| upstream 2:1 BPVE2³ × upstream 5:0 ETFA³ | −3.79 | 0.11 | (0.31) | 0.00 | (0.00) | 0 |
| upstream 2:0 LINE_L2¹ × upstream 7:6 Pit1³ | −3.78 | 0.13 | (0.46) | 0.00 | (0.00) | 0 |
| upstream 2:1 TFIID³ × downstream 0:100 MLT1C phase | −3.77 | 0.24 | (0.73) | 0.00 | (0.00) | 0 |
| change² | | | | | | |
| upstream 7:6 Sp1³ | 3.76 | 0.20 | (0.40) | 0.35 | (0.48) | 2.81 × 10⁻² |
| downstream 5:10 L1PB±¹ | −3.75 | 0.00 | (0.28) | −0.03 | (0.58) | 3.95 × 10⁻¹ |
| upstream 7:6 GTIIC³ × upstream 5:0 CP1³ | −3.73 | 0.06 | (0.24) | 0.00 | (0.00) | 0 |
| upstream 2:0 DNA_MER1_type¹ | −3.72 | 0.13 | (0.44) | 0.03 | (0.16) | 7.19 × 10⁻⁵ |
| upstream 2:1 SRF¹ | −3.71 | 0.02 | (0.13) | 0.00 | (0.00) | 0 |
| upstream 4:3 Pit1³ × downstream 0:100 MLT1C phase | −3.7 | 0.18 | (0.63) | 0.00 | (0.00) | 0 |
| change² | | | | | | |
| upstream 2:0 E4TF1¹ | 3.69 | 0.21 | (0.48) | 0.13 | (0.46) | 1.32 × 10⁻¹ |
| upstream 9:8 NFuE5¹ × upstream 6:5 Oct1³ | −3.68 | 0.14 | (0.42) | 0.00 | (0.00) | 0 |
| upstream 10:0 CpGi^{1,11} | 3.67 | 2.32 | (2.09) | 2.48 | (1.97) | 3.14 × 10⁻¹ |
| downstream 0:2 SINE_MIR¹ | 3.66 | 0.32 | (0.66) | 0.25 | (0.59) | 2.16 × 10⁻¹ |
| upstream 7:6 SIF³ | 3.65 | 0.22 | (0.41) | 0.30 | (0.46) | 1.31 × 10⁻¹ |
| upstream 5:4 E4F1¹ × upstream 6:5 APF³ | −3.63 | 0.17 | (0.50) | 0.00 | (0.00) | 0 |
| upstream 7:6 AP1³ × upstream 2:1 E4TF1³ | −3.62 | 0.06 | (0.25) | 0.00 | (0.00) | 0 |
| upstream 5:4 NFuE5³ × upstream 4:3 Pit1³ | −3.61 | 0.11 | (0.31) | 0.00 | (0.00) | 0 |
| downstream 10:100 L1PA¹ | −3.6 | 2.76 | (3.56) | 3.03 | (3.09) | 2.95 × 10⁻¹ |
| upstream 4:3 CEBP³ | 3.59 | 0.04 | (0.19) | 0.10 | (0.30) | 9.64 × 10⁻² |
| upstream 5:0 L1M² | 3.58 | 0.01 | (0.21) | 0.06 | (0.36) | 1.96 × 10⁻¹ |
| upstream 10:5 MIR3¹ | −3.57 | 0.14 | (0.41) | 0.08 | (0.27) | 6.80 × 10⁻² |
| downstream 0:5 FAM¹ | −3.56 | 0.01 | (0.13) | 0.00 | (0.00) | 0 |
| downstream 5:10 L1MD±² | −3.55 | 26.33 | (197.25) | 24.09 | (152.26) | 4.64 × 10⁻¹ |
| upstream 3:2 Pit1¹ × upstream 7:6 BPVE2³ | −3.54 | 0.25 | (0.77) | 0.00 | (0.00) | 0 |
| downstream 0:2 FLAM±¹ | 3.53 | 0.00 | (0.22) | 0.03 | (0.16) | 2.08 × 10⁻¹ |
| upstream 2:0 SINE_MIR±² × upstream 8:7 GT2B³ | −3.52 | 13.67 | (50.16) | 0.00 | (0.00) | 0 |
| upstream 2:0 DNA_MER1_type¹ × upstream 8:7 ICSBP³ | −3.51 | 0.10 | (0.39) | 0.00 | (0.00) | 0 |
| upstream 6:5 ETFA³ | 3.5 | 0.06 | (0.23) | 0.10 | (0.30) | 1.85 × 10⁻¹ |
| downstream 0:1 L1M±¹ | −3.49 | 0.00 | (0.02) | −0.03 | (0.16) | 1.66 × 10⁻¹ |
| downstream 0:1 CpGi¹ | −3.48 | 0.22 | (0.47) | 0.25 | (0.49) | 3.67 × 10⁻¹ |
| downstream 0:100 MLT1C phase change² | −3.47 | 0.12 | (0.25) | 0.06 | (0.20) | 3.07 × 10⁻² |
| upstream 10:0 GTIIC³ × upstream 10:0 Oct1³ | 3.46 | 0.92 | (0.26) | 1.00 | (0.00) | 0 |
| upstream 9:8 Sp1³ | −3.45 | 0.20 | (0.40) | 0.10 | (0.30) | 2.67 × 10⁻² |
| upstream 3:2 AP1¹ × downstream 0:100 MLT1C phase | −3.44 | 0.78 | (2.91) | 0.03 | (0.16) | 0 |
| change² | | | | | | |
| upstream 10:5 LINE_L1² | −3.43 | 5.05 | (8.09) | 5.06 | (8.30) | 4.97 × 10⁻¹ |
| upstream 10:5 AluJ±² × upstream 10:0 CP1³ | −3.42 | 44.66 | (129.92) | 2.10 | (13.28) | 0 |
| upstream 100:10 L1P±² | −3.41 | 230.89 | (832.85) | 61.10 | (160.04) | 3.76 × 10⁻⁸ |
| upstream 90:80 CpGi^{1,10} | −3.4 | 0.35 | (0.83) | 0.28 | (0.64) | 2.33 × 10⁻¹ |
| upstream 6:5 AP2³ × upstream 3:2 Pit1³ | −3.39 | 0.09 | (0.28) | 0.00 | (0.00) | 0 |
| upstream 100:10 L1MB±¹ | −3.38 | 0.14 | (3.76) | −0.51 | (3.60) | 1.35 × 10⁻¹ |
| downstream 10:100 LTR_ERV1¹ | −3.37 | 6.24 | (7.74) | 6.30 | (7.51) | 4.80 × 10⁻¹ |
| downstream 10:100 FRAM±² × upstream 8:7 SIF³ | −3.36 | 18.57 | (67.07) | 0.07 | (0.36) | 0 |
| upstream 4:3 ETFA¹ | 3.35 | 0.06 | (0.24) | 0.10 | (0.30) | 1.96 × 10⁻¹ |
| upstream 6:5 PU1¹ | 3.34 | 0.89 | (1.05) | 1.10 | (1.19) | 1.34 × 10⁻¹ |
| downstream 0:5 L1MC±² | −3.33 | 53.61 | (230.70) | 98.35 | (454.18) | 2.71 × 10⁻¹ |
| upstream 1:0 L1MA¹ | −3.32 | 0.01 | (0.12) | 0.00 | (0.00) | 0 |
| upstream 4:3 NFuE5³ × upstream 2:1 Pit1³ | −3.31 | 0.10 | (0.30) | 0.00 | (0.00) | 0 |
| upstream 2:1 NFIII¹ × upstream 10:9 E4F1³ | −3.3 | 0.36 | (1.09) | 0.00 | (0.00) | 0 |
| ditan10kup | −3.29 | 23.10 | (54.47) | 15.50 | (23.28) | 2.46 × 10⁻² |
| upstream 2:0 NFkB³ | 3.28 | 0.18 | (0.38) | 0.13 | (0.33) | 1.64 × 10⁻¹ |
| upstream 10:0 IgPE2¹ | 3.27 | 0.20 | (0.46) | 0.25 | (0.49) | 2.73 × 10⁻¹ |
| upstream 9:8 GATA1³ × upstream 2:0 NFkB³ | −3.26 | 0.08 | (0.27) | 0.00 | (0.00) | 0 |
| upstream 9:8 NFuE5¹ × upstream 6:5 Oct1¹ | −3.25 | 0.18 | (0.64) | 0.00 | (0.00) | 0 |
| downstream 5:10 AluY¹ | 3.24 | 0.37 | (0.69) | 0.50 | (0.75) | 1.44 × 10⁻¹ |
| upstream 4:3 PU1¹ | 3.23 | 0.88 | (1.04) | 1.13 | (1.20) | 1.09 × 10⁻¹ |
| upstream 10:5 LTR_MaLR±² × upstream 1:0 MLTF³ | −3.22 | 68.32 | (203.70) | 0.08 | (0.51) | 0 |
| upstream 2:1 E4TF1³ × upstream 10:0 NFuE5³ | −3.21 | 0.07 | (0.25) | 0.00 | (0.00) | 0 |
| upstream 10:5 L1M3¹ | 3.2 | 0.01 | (0.14) | 0.08 | (0.47) | 1.96 × 10⁻¹ |
| upstream 10:9 E4F1³ × upstream 3:2 TFIID³ | −3.19 | 0.11 | (0.31) | 0.00 | (0.00) | 0 |
| upstream 100:10 MIR±¹ | 3.18 | 0.10 | (2.33) | 0.31 | (3.55) | 3.63 × 10⁻¹ |
| upstream 9:8 GATA1¹ | 3.17 | 0.68 | (0.89) | 0.78 | (1.07) | 2.88 × 10⁻¹ |
| upstream 10:9 SIF¹ | −3.16 | 0.27 | (0.65) | 0.28 | (0.55) | 4.59 × 10⁻¹ |
| upstream 100:10 HAL1±² | −3.15 | 221.71 | (473.89) | 119.00 | (233.60) | 4.63 × 10⁻³ |
| upstream 100:10 LTR_ERVL² | 3.14 | 1.06 | (1.45) | 1.89 | (2.96) | 4.46 × 10⁻² |
| upstream 2:0 LINE_L2¹ × upstream 6:5 Oct1³ | −3.13 | 0.13 | (0.47) | 0.00 | (0.00) | 0 |
| upstream 9:8 APF¹ | −3.12 | 2.42 | (1.99) | 1.65 | (1.48) | 1.18 × 10⁻³ |
| downstream 0:2 AluY±¹ | −3.11 | 0.01 | (0.37) | −0.08 | (0.27) | 3.23 × 10⁻² |
| upstream 10:5 DNA_AcHobo² | 3.1 | 0.06 | (0.51) | 0.22 | (1.24) | 2.09 × 10⁻¹ |
| upstream 3:2 SIF³ × upstream 5:0 NFkB³ | −3.09 | 0.08 | (0.27) | 0.00 | (0.00) | 0 |
| upstream 100:10 MIR3±² | −3.08 | 71.24 | (118.59) | 35.53 | (72.54) | 1.95 × 10⁻³ |
| downstream 5:10 L1MB±² | −3.07 | 60.96 | (281.86) | 84.80 | (423.47) | 3.64 × 10⁻¹ |
| upstream 10:0 IgPE2³ | 3.06 | 0.18 | (0.38) | 0.23 | (0.42) | 2.59 × 10⁻¹ |
| downstream 10:100 LINE_CR1¹ × upstream 3:2 Pit1³ | −3.05 | 0.58 | (1.31) | 0.10 | (0.30) | 1.47 × 10⁻¹² |
| upstream 2:0 NFkB¹ × upstream 5:4 TFIID³ | −3.04 | 0.12 | (0.37) | 0.00 | (0.00) | 0 |
| upstream 1:0 SRF¹ | −3.03 | 0.01 | (0.12) | 0.00 | (0.00) | 0 |
| upstream 10:5 LINE_L1¹ × downstream 0:100 MLT1C | −3.02 | 0.61 | (2.34) | 0.00 | (0.00) | 0 |
| phase change² | | | | | | |
| upstream 5:0 CpGi² | 3.01 | 91.87 | (92.76) | 126.55 | (121.01) | 4.07 × 10⁻² |
| upstream 4:3 SIF¹ | −3 | 0.27 | (0.68) | 0.13 | (0.33) | 5.44 × 10⁻³ |
| upstream 100:10 LINE_CR1±¹ | −2.99 | 0.07 | (1.61) | −0.11 | (1.11) | 1.52 × 10⁻¹ |
| downstream 0:2 L1MB±¹ | −2.98 | 0.00 | (0.26) | −0.05 | (0.22) | 8.95 × 10⁻² |
| downstream 5:10 DNA_AcHobo±¹ | 2.97 | 0.00 | (0.22) | 0.03 | (0.16) | 1.73 × 10⁻¹ |
| downstream 0:2 LINE_L2¹ × upstream 8:7 ICSBP¹ | −2.96 | 0.32 | (1.07) | 0.00 | (0.00) | 0 |
| upstream 9:8 SIF¹ × upstream 1:0 ATF³ | −2.95 | 0.15 | (0.49) | 0.00 | (0.00) | 0 |
| upstream 8:7 NFkB³ | −2.94 | 0.07 | (0.26) | 0.05 | (0.22) | 2.54 × 10⁻¹ |
| upstream 4:3 IgPE2¹ | 2.93 | 0.02 | (0.14) | 0.03 | (0.16) | 4.22 × 10⁻¹ |
| Motif₇⁹ | −2.92 | 0.00 | (0.17) | −0.05 | (0.22) | 7.52 × 10⁻² |
| upstream 2:0 ETFA³ × downstream 0:100 MIR phase | −2.91 | 0.06 | (0.16) | 0.00 | (0.00) | 0 |
| change² | | | | | | |
| upstream 9:8 NFuE5¹ | −2.9 | 0.34 | (0.63) | 0.25 | (0.54) | 1.49 × 10⁻¹ |
| downstream 10:100 LTR_ERV¹ | −2.89 | 0.03 | (0.24) | 0.03 | (0.16) | 3.57 × 10⁻¹ |
| upstream 6:5 AP2¹ | 2.88 | 0.38 | (0.79) | 0.65 | (1.00) | 5.07 × 10⁻² |
| upstream 5:4 TFIID¹ × upstream 2:0 NFkB³ | −2.87 | 0.20 | (0.72) | 0.00 | (0.00) | 0 |
| downstream 10:100 FRAM±² × upstream 5:0 GATA1¹ | −2.86 | 235.74 | (440.52) | 35.12 | (90.45) | 0 |
| downstream 10:100 LINE_CR1² × upstream 100:10 | −2.85 | 0.77 | (1.91) | 0.06 | (0.19) | 0 |
| MIR3¹ | | | | | | |
| upstream 4:3 BPVE2³ × downstream 0:100 MLT1C phase | −2.84 | 0.04 | (0.16) | 0.00 | (0.00) | 0 |
| change² | | | | | | |
| upstream 60:50 CpGi^{1,10} | 2.83 | 0.35 | (0.80) | 0.53 | (1.13) | 1.71 × 10⁻¹ |
| downstream 5:10 MIR3¹ | 2.82 | 0.14 | (0.41) | 0.20 | (0.41) | 1.88 × 10⁻¹ |
| upstream 5:0 L1PA±¹ | 2.81 | 0.01 | (0.35) | 0.05 | (0.22) | 1.12 × 10⁻¹ |
| upstream 100:10 LTR_ERV1² | 2.8 | 2.85 | (3.93) | 3.68 | (6.61) | 2.19 × 10⁻¹ |
| upstream 10:9 NFuE5¹ × upstream 10:9 Oct1¹ | −2.79 | 0.17 | (2.41) | 0.00 | (0.00) | 0 |
| upstream 10:5 DNA_MER1_type² × upstream 5:4 Pit1³ | −2.78 | 0.28 | (1.09) | 0.00 | (0.00) | 0 |
| downstream 10:100 AluJ±¹ | 2.77 | −0.08 | (2.42) | 0.05 | (2.65) | 3.78 × 10⁻¹ |
| downstream 10:100 LINE_CR1¹ × upstream 9:8 TFIID³ | −2.76 | 0.84 | (1.50) | 0.10 | (0.30) | 0 |
| upstream 2:0 SINE_MIR² × upstream 10:9 BPVE2³ | −2.75 | 1.03 | (3.37) | 0.00 | (0.00) | 0 |
| upstream 9:8 TFIID³ × upstream 2:0 ICP4³ | −2.74 | 0.08 | (0.27) | 0.00 | (0.00) | 0 |
| upstream 2:1 BPVE2¹ × upstream 6:5 Pit1³ | −2.73 | 0.21 | (0.52) | 0.00 | (0.00) | 0 |
| exon CpGi² | 2.72 | 161.59 | (186.01) | 272.77 | (250.19) | 4.22 × 10⁻³ |
| upstream 100:10 FAM² × upstream 6:5 Pit1³ | −2.71 | 0.02 | (0.06) | 0.00 | (0.00) | 0 |
| downstream 5:10 AluS² × upstream 1:0 MLTF³ | −2.7 | 3.47 | (5.11) | 0.34 | (0.92) | 0 |
| upstream 2:0 PEA2¹ | 2.69 | 0.07 | (0.28) | 0.08 | (0.27) | 4.77 × 10⁻¹ |
| upstream 3:2 ICP4³ | 2.68 | 0.05 | (0.22) | 0.03 | (0.16) | 1.59 × 10⁻¹ |
| upstream 100:10 SINE_MIR±¹ | 2.67 | 0.10 | (2.22) | 0.33 | (3.43) | 3.36 × 10⁻¹ |
| upstream 10:5 FAM±¹ | −2.66 | 0.00 | (0.13) | −0.03 | (0.16) | 1.61 × 10⁻¹ |
| upstream 10:5 L1PB² | −2.65 | 0.15 | (1.51) | 0.00 | (0.00) | 0 |
| upstream 5:0 LTR_ERV1±² × upstream 4:3 TFIID³ | −2.64 | 58.25 | (241.18) | 1.42 | (8.07) | 0 |
| upstream 100:10 CpGi¹ × upstream 100:10 AluS² | −2.63 | 37.02 | (53.50) | 12.01 | (11.29) | 0 |
| downstream 0:5 SINE_MIR¹ | 2.62 | 0.86 | (1.21) | 0.65 | (0.83) | 6.03 × 10⁻² |
| upstream 2:0 Sp1³ | 2.61 | 0.62 | (0.49) | 0.73 | (0.45) | 7.79 × 10⁻² |
| upstream 8:7 AP1³ × upstream 7:6 NF1³ | −2.6 | 0.10 | (0.29) | 0.00 | (0.00) | 0 |
| upstream 8:7 GT2B¹ × upstream 5:4 NFuE5³ | −2.59 | 0.13 | (0.45) | 0.00 | (0.00) | 0 |
| downstream 0:1 LINE_CR1² | 2.58 | 0.19 | (2.17) | 0.47 | (2.97) | 2.78 × 10⁻¹ |
| downstream 0:5 AluS¹ × upstream 3:2 TFIID¹ | −2.57 | 1.83 | (3.74) | 0.28 | (0.82) | 4.77 × 10⁻¹⁵ |
| upstream 100:10 AluJ² × upstream 10:9 NFuE5³ | −2.56 | 1.11 | (2.27) | 0.12 | (0.37) | 0 |
| upstream 2:0 NFuE3³ | −2.55 | 0.11 | (0.31) | 0.10 | (0.30) | 4.15 × 10⁻¹ |
| upstream 10:5 DNA_MER1_type¹ × upstream 5:4 Pit1¹ | −2.54 | 0.26 | (1.08) | 0.00 | (0.00) | 0 |
| upstream 1:0 PU1¹ | −2.53 | 1.00 | (1.09) | 0.73 | (0.85) | 2.39 × 10⁻² |
| downstream 0:5 FLAM±¹ | −2.52 | 0.01 | (0.38) | −0.08 | (0.35) | 7.23 × 10⁻² |
| upstream 100:10 CpGi¹ × upstream 100:10 SINE_Alu² | −2.51 | 60.28 | (85.57) | 22.82 | (21.91) | 1.48 × 10⁻¹³ |
| upstream 10:5 Alu¹ | −2.5 | 0.02 | (0.16) | 0.00 | (0.00) | 0 |
| downstream 10:100 CpGi³ × upstream 2:0 PEA1¹ | −2.49 | 0.08 | (0.32) | 0.00 | (0.00) | 0 |
| upstream 2:0 NFuE3¹ | −2.48 | 0.12 | (0.38) | 0.10 | (0.30) | 3.18 × 10⁻¹ |
| upstream 8:7 SRF¹ | 2.47 | 0.01 | (0.12) | 0.03 | (0.16) | 3.27 × 10⁻¹ |
| upstream 7:6 APF³ | 2.46 | 0.83 | (0.37) | 0.88 | (0.33) | 2.22 × 10⁻¹ |
| upstream 1:0 MLTF¹ | −2.45 | 1.23 | (1.32) | 0.63 | (0.90) | 6.82 × 10⁻⁵ |
| upstream 10:0 CpGi^{1,10} | 2.44 | 0.38 | (0.86) | 0.63 | (0.95) | 5.63 × 10⁻² |
| downstream 10:100 DNA_Mariner¹ × upstream 9:8 Pit1¹ | −2.43 | 0.33 | (1.34) | 0.00 | (0.00) | 0 |
| downstream 0:5 LTR_MaLR¹ × upstream 2:0 BPVE2¹ | −2.42 | 0.37 | (1.23) | 0.00 | (0.00) | 0 |
| upstream 100:10 DNA_Tc2² | 2.41 | 0.04 | (0.13) | 0.11 | (0.43) | 1.47 × 10⁻¹ |
| upstream 100:10 AluJ² × upstream 10:9 E4F1¹ | −2.4 | 0.72 | (2.14) | 0.04 | (0.14) | 0 |
| upstream 7:6 NF1¹ × upstream 7:6 AP1³ | −2.39 | 0.11 | (0.36) | 0.00 | (0.00) | 0 |
| upstream 100:10 CpGi² × upstream 100:10 AluS² | −2.38 | 601.34 | (769.54) | 185.70 | (151.99) | 0 |
| upstream 5:4 E4F1³ × upstream 1:0 MLTF³ | −2.37 | 0.12 | (0.32) | 0.00 | (0.00) | 0 |
| upstream 4:3 TFIID³ × downstream 0:100 MLT1C phase | −2.36 | 0.25 | (0.72) | 0.00 | (0.00) | 0 |
| change² | | | | | | |
| downstream 10:100 LINE_CR1² × upstream 100:10 | −2.35 | 1.70 | (3.44) | 0.31 | (0.63) | 0 |
| DNA_MER1_type¹ | | | | | | |
| upstream 10:5 LTR_MaLR±² × upstream 1:0 MLTF¹ | −2.34 | 129.34 | (473.41) | 0.08 | (0.51) | 0 |
| upstream 4:3 AP1¹ × upstream 5:4 E4F1³ | −2.33 | 0.33 | (0.91) | 0.00 | (0.00) | 0 |
| upstream 9:8 ICSBP¹ | 2.32 | 1.50 | (1.21) | 1.35 | (1.05) | 1.89 × 10⁻¹ |
| upstream 2:0 ETFA¹ × upstream 10:9 MLTF³ | −2.31 | 0.10 | (0.34) | 0.00 | (0.00) | 0 |
| upstream 10:5 L1PA¹ | −2.3 | 0.12 | (0.45) | 0.13 | (0.46) | 4.87 × 10⁻¹ |
| upstream 10:5 FRAM±² | 2.29 | 8.63 | (36.14) | 7.60 | (33.59) | 4.25 × 10⁻¹ |
| upstream 100:10 DNA_Tc2±¹ | −2.28 | 0.00 | (0.63) | 0.07 | (0.77) | 2.86 × 10⁻¹ |
| upstream 1:0 NFuE5³ | 2.27 | 0.20 | (0.40) | 0.15 | (0.36) | 1.96 × 10⁻¹ |
| upstream 2:0 MIR² × upstream 10:9 BPVE2³ | −2.26 | 0.88 | (3.06) | 0.00 | (0.00) | 0 |
| upstream 100:10 MIR3±² × upstream 1:0 MLTF¹ | −2.25 | 87.83 | (235.51) | 11.73 | (37.57) | 3.33 × 10⁻¹⁶ |
| upstream 100:10 DNA_MER1_type² | 2.24 | 1.12 | (0.84) | 1.23 | (0.97) | 2.45 × 10⁻¹ |
| downstream 10:100 FAM¹ × upstream 4:3 TFIID¹ | −2.23 | 0.30 | (1.03) | 0.00 | (0.00) | 0 |
| upstream 10:5 LTR_MaLR±² × upstream 2:0 BPVE2³ | −2.22 | 62.93 | (194.19) | 0.00 | (0.00) | 0 |
| downstream 10:100 LINE_CR1² × upstream 100:10 | −2.21 | 0.60 | (1.19) | 0.07 | (0.17) | 0 |
| SINE_MIR² | | | | | | |
| upstream 2:0 DNA_MER1_type¹ × upstream 5:0 Pit1¹ | −2.2 | 0.45 | (2.01) | 0.00 | (0.00) | 0 |
| downstream 0:2 LINE_CR1² | 2.19 | 0.20 | (1.82) | 0.83 | (5.23) | 2.31 × 10⁻¹ |
| downstream 10:100 LINE_CR1² × upstream 9:8 APF³ | −2.18 | 0.20 | (0.34) | 0.04 | (0.10) | 2.76 × 10⁻¹³ |
| downstream 10:100 LINE_CR1² × downstream 5:10 | −2.17 | 0.26 | (0.72) | 0.03 | (0.07) | 0 |
| SINE_MIR¹ | | | | | | |
| intron L1P¹ | −2.16 | 0.13 | (0.61) | 0.03 | (0.16) | 1.61 × 10⁻⁴ |
| upstream 8:7 GT2B³ × upstream 2:0 NFkB³ | −2.15 | 0.06 | (0.24) | 0.00 | (0.00) | 0 |
| upstream 5:0 L1MA±² | −2.14 | 30.55 | (188.23) | 0.00 | (0.00) | 0 |
| downstream 0:5 DNA_MER2_type±² | −2.13 | 40.07 | (209.05) | 7.73 | (48.86) | 1.02 × 10⁻⁴ |
| exon LTR_ERV1¹ | −2.12 | 0.01 | (0.16) | 0.00 | (0.00) | 0 |
| upstream 5:0 AluY² × upstream 9:8 Pit1³ | −2.11 | 0.75 | (2.43) | 0.00 | (0.00) | 0 |
| downstream 10:100 FRAM±² × upstream 5:0 TFIID¹ | −2.1 | 411.51 | (770.95) | 93.55 | (208.60) | 4.38 × 10⁻¹² |
| intron LINE_L1±¹ | −2.09 | −1.28 | (3.60) | −1.64 | (3.66) | 2.68 × 10⁻¹ |
| downstream 0:5 AluJ¹ × upstream 4:3 GTIIC³ | −2.08 | 0.17 | (0.59) | 0.00 | (0.00) | 0 |
| downstream 10:100 LTR_MaLR¹ | −2.07 | 8.71 | (7.00) | 7.75 | (5.15) | 1.26 × 10⁻¹ |
| downstream 0:2 MIR¹ × upstream 1:0 ATF³ | −2.06 | 0.14 | (0.45) | 0.00 | (0.00) | 0 |
| upstream 5:0 DNA_AcHobo¹ | 2.05 | 0.04 | (0.24) | 0.03 | (0.16) | 3.17 × 10⁻¹ |
| upstream 100:10 Other¹ × upstream 100:0 MIR phase | −2.04 | 0.07 | (0.23) | 0.00 | (0.00) | 0 |
| change² | | | | | | |
| intron MIR±² × upstream 6:5 APF³ | −2.03 | 36.15 | (112.42) | 0.72 | (1.16) | 0 |
| upstream 100:10 Other¹ × upstream 5:4 ICSBP³ | −2.02 | 0.14 | (0.48) | 0.00 | (0.00) | 0 |
| downstream 0:2 DNA² | −2.01 | 0.06 | (1.08) | 0.00 | (0.00) | 0 |
| upstream 2:1 BPVE2¹ × upstream 4:3 ATF³ | −2 | 0.18 | (0.49) | 0.00 | (0.00) | 0 |
| upstream 7:6 NF1¹ × upstream 2:0 ATF³ | −1.99 | 0.09 | (0.33) | 0.00 | (0.00) | 0 |
| upstream 5:0 DNA_MER2_type² | −1.98 | 0.60 | (2.74) | 0.36 | (2.27) | 2.56 × 10⁻¹ |
| upstream 4:3 Pit1¹ | −1.97 | 0.70 | (1.20) | 0.50 | (0.88) | 7.67 × 10⁻² |
| downstream 10:100 L1M3±¹ | 1.96 | 0.00 | (0.71) | 0.13 | (1.22) | 2.66 × 10⁻¹ |
| upstream 2:0 NFuE4¹ × upstream 5:0 GATA1³ | −1.95 | 0.07 | (0.28) | 0.00 | (0.00) | 0 |
| upstream 5:0 E2F¹ | −1.94 | 0.09 | (0.31) | 0.10 | (0.30) | 4.17 × 10⁻¹ |
| downstream 0:2 SINE_MIR¹ × upstream 1:0 ATF³ | −1.93 | 0.17 | (0.51) | 0.00 | (0.00) | 0 |
| upstream 8:7 SIF³ | −1.92 | 0.21 | (0.41) | 0.13 | (0.33) | 5.63 × 10⁻² |
| upstream 5:4 PEA1¹ × upstream 3:2 APF³ | −1.91 | 0.09 | (0.31) | 0.00 | (0.00) | 0 |
| downstream 10:100 AluY² × downstream 0:2 LINE_L2¹ | −1.9 | 0.39 | (1.33) | 0.01 | (0.05) | 0 |
| downstream 10:100 Alu±¹ | 1.89 | 0.02 | (0.58) | 0.00 | (0.55) | 4.10 × 10⁻¹ |
| upstream 5:0 AluY² × upstream 6:5 PU1³ | −1.88 | 1.03 | (2.86) | 0.00 | (0.00) | 0 |
| upstream 100:10 FRAM¹ | 1.87 | 1.09 | (1.29) | 0.88 | (1.28) | 1.56 × 10⁻¹ |
| downstream 0:2 SINE_MIR² × upstream 1:0 ATF³ | −1.86 | 1.08 | (3.30) | 0.00 | (0.00) | 0 |
| upstream 3:2 TFIID¹ | −1.85 | 1.14 | (1.29) | 0.85 | (1.12) | 5.99 × 10⁻² |
| upstream 10:9 ICP4³ | 1.84 | 0.05 | (0.21) | 0.08 | (0.27) | 2.70 × 10⁻¹ |
| upstream 5:0 L1PA¹ | 1.83 | 0.09 | (0.37) | 0.10 | (0.44) | 4.59 × 10⁻¹ |
| downstream 10:100 L1MC² | −1.82 | 1.41 | (1.55) | 1.43 | (1.64) | 4.69 × 10⁻¹ |
| upstream 9:8 TFIID³ × upstream 8:7 Sp1³ | −1.81 | 0.10 | (0.30) | 0.00 | (0.00) | 0 |
| intron L1PB¹ | 1.8 | 0.26 | (1.11) | 0.45 | (2.25) | 2.98 × 10⁻¹ |
| upstream 10:5 SINE_MIR² × upstream 5:0 AluS¹ | −1.79 | 2.08 | (4.66) | 0.23 | (0.63) | 0 |
| upstream 5:0 AluS² × upstream 2:0 AP2³ | −1.78 | 5.68 | (9.31) | 1.28 | (2.82) | 2.27 × 10⁻¹² |
| exon LIME² | 1.77 | 0.23 | (3.77) | 3.17 | (20.06) | 1.83 × 10⁻¹ |
| upstream 10:5 FAM² | 1.76 | 0.02 | (0.20) | 0.03 | (0.22) | 3.89 × 10⁻¹ |
| upstream 1:0 AP1¹ × upstream 10:9 NFuE5³ | −1.75 | 0.40 | (0.94) | 0.03 | (0.16) | 0 |
| upstream 10:5 SINE_MIR±² | 1.74 | 63.22 | (108.32) | 53.71 | (117.12) | 3.08 × 10⁻¹ |
| upstream 4:3 AP2³ | 1.73 | 0.26 | (0.44) | 0.38 | (0.49) | 8.00 × 10⁻² |
| upstream 100:10 AluJ² × upstream 70:60 CpGi^{1,11} | −1.72 | 7.82 | (11.89) | 2.40 | (3.04) | 3.97 × 10⁻¹⁴ |
| upstream 100:10 AluY±¹ | −1.71 | 0.29 | (2.38) | 0.09 | (1.98) | 2.70 × 10⁻¹ |
| downstream 10:100 LINE_CR1¹ × upstream 10:5 AluS±² | −1.7 | 249.42 | (708.04) | 23.28 | (81.55) | 0 |
| upstream 2:0 BPVE2¹ × upstream 5:0 NFuE5¹ | −1.69 | 1.57 | (2.87) | 0.28 | (0.55) | 0 |
| upstream 5:0 DNA_Tc2¹ | 1.68 | 0.01 | (0.13) | 0.05 | (0.32) | 2.13 × 10⁻¹ |
| upstream 2:0 ETFA³ × downstream 0:100 MIR phase | −1.67 | 1.16 | (3.68) | 0.00 | (0.00) | 0 |
| change¹ | | | | | | |
| upstream 3:2 BPVE2¹ | 1.66 | 0.48 | (0.71) | 0.58 | (0.84) | 2.54 × 10⁻¹ |
| downstream 0:5 DNA_Mariner±¹ | 1.65 | 0.00 | (0.18) | 0.00 | (0.23) | 4.81 × 10⁻¹ |
| upstream 100:10 DNA_T2_type±² | −1.64 | 12.81 | (88.09) | 0.00 | (0.00) | 0 |
| downstream 0:1 L1ME¹ | 1.63 | 0.03 | (0.21) | 0.08 | (0.35) | 2.27 × 10⁻¹ |
| upstream 100:10 DNA_Tip100² | −1.62 | 0.12 | (0.30) | 0.10 | (0.35) | 3.51 × 10⁻¹ |
| upstream 5:0 Pit1¹ × downstream 0:100 MLT1C phase | −1.61 | 0.43 | (1.24) | 0.04 | (0.17) | 0 |
| change² | | | | | | |
| upstream 10:5 AluJ² × upstream 2:0 BPVE2³ | −1.6 | 1.27 | (2.41) | 0.02 | (0.13) | 0 |
| exon SINE_Alu±¹ | −1.59 | 0.00 | (0.43) | −0.05 | (0.22) | 7.73 × 10⁻² |
| upstream 2:0 L1M2¹ | −1.58 | 0.01 | (0.11) | 0.03 | (0.16) | 2.52 × 10⁻¹ |
| intron DNA_MER2_type² | −1.57 | 40.83 | (121.55) | 36.63 | (89.65) | 3.86 × 10⁻¹ |
| upstream 5:0 NFuE1³ | 1.56 | 0.20 | (0.40) | 0.25 | (0.44) | 2.55 × 10⁻¹ |
| downstream 10:100 FLAM² | 1.55 | 0.34 | (0.32) | 0.22 | (0.23) | 1.57 × 10⁻³ |
| upstream 5:0 L1ME¹ × upstream 10:0 NF1¹ | −1.54 | 0.38 | (1.64) | 0.00 | (0.00) | 0 |
| upstream 5:0 AluY¹ × upstream 100:0 MIR phase | −1.53 | 0.14 | (0.29) | 0.01 | (0.07) | 7.22 × 10⁻¹⁵ |
| change² | | | | | | |
| upstream 5:4 APF³ | 1.52 | 0.84 | (0.37) | 0.80 | (0.41) | 2.95 × 10⁻¹ |
| upstream 10:5 LTR_MaLR¹ × upstream 2:0 BPVE2³ | −1.51 | 0.31 | (0.84) | 0.00 | (0.00) | 0 |
| intron MIR±² × upstream 5:0 AP2³ | −1.5 | 33.71 | (108.04) | 0.77 | (1.32) | 0 |
| upstream 2:1 COUP³ × upstream 2:0 NFuE4³ | −1.49 | 0.07 | (0.25) | 0.00 | (0.00) | 0 |
| intron MIR±² × upstream 7:6 NFIII³ | −1.48 | 34.33 | (109.61) | 0.78 | (1.20) | 0 |
| downstream 0:5 AluJ² × upstream 5:4 SIF³ | −1.47 | 0.88 | (3.00) | 0.00 | (0.00) | 0 |
| upstream 2:0 AluS² × upstream 1:0 Pit1³ | −1.46 | 2.32 | (7.10) | 0.00 | (0.00) | 0 |
| upstream 6:5 NFuE3¹ | 1.45 | 0.08 | (0.29) | 0.13 | (0.33) | 1.87 × 10⁻¹ |
| downstream 0:5 SINE_Alu¹ × upstream 5:4 SIF³ | −1.44 | 0.78 | (2.14) | 0.05 | (0.22) | 0 |
| upstream 5:0 AluY¹ × upstream 2:0 PU1³ | −1.43 | 0.28 | (0.61) | 0.03 | (0.16) | 8.00 × 10⁻¹³ |
| downstream 0:2 SINE_MIR±¹ | −1.42 | 0.00 | (0.64) | 0.00 | (0.51) | 4.88 × 10⁻¹ |
| upstream 10:5 L1MD¹ | −1.41 | 0.08 | (0.48) | 0.00 | (0.00) | 0 |
| intron MIR±² × upstream 9.04:8.99 nucleosome | −1.4 | 17.79 | (52.21) | 1.61 | (8.15) | 7.77 × 10⁻¹⁶ |
| potential^{sd} | | | | | | |
| upstream 100:10 AluS² × upstream 10:9 NFuE5³ | −1.39 | 3.02 | (6.15) | 0.41 | (1.13) | 0 |
| upstream 100:10 AluS¹ × upstream 100:10 MIR3±² | −1.38 | 2450.40 | (5083.01) | 449.61 | (1068.36) | 7.55 × 10⁻¹⁵ |
| downstream 0:1 AluS² × upstream 4:3 PU1³ | −1.37 | 3.83 | (11.21) | 0.00 | (0.00) | 0 |
| upstream 5:0 CpGi¹ × upstream 2:0 AluJ¹ | −1.36 | 0.12 | (0.48) | 0.00 | (0.00) | 0 |
| upstream 2:0 AluJ² × upstream 10:9 PU1³ | −1.35 | 1.57 | (4.94) | 0.00 | (0.00) | 0 |
| exon MIR3² | −1.34 | 0.20 | (1.75) | 0.00 | (0.00) | 0 |
| downstream 0:5 AluJ±² × upstream 10:0 NFuE4³ | −1.33 | 36.43 | (116.04) | 0.00 | (0.00) | 0 |
| downstream 10:100 CpGi¹ | 1.32 | 21.32 | (16.93) | 22.00 | (14.76) | 3.87 × 10⁻¹ |
| downstream 10:100 DNA_Mariner¹ × upstream 100:10 | −1.31 | 2.71 | (8.90) | 0.00 | (0.00) | 0 |
| LTR_ERV1¹ | | | | | | |
| downstream 0:5 AluJ² × upstream 7:6 GTIIC¹ | −1.3 | 1.01 | (3.61) | 0.00 | (0.00) | 0 |
| upstream 9:8 COUP¹ | 1.29 | 1.97 | (1.44) | 1.90 | (1.68) | 3.94 × 10⁻¹ |
| upstream 10:5 L1MD² | −1.28 | 0.28 | (1.95) | 0.00 | (0.00) | 0 |
| upstream 10:9 NFuE5¹ × upstream 1:0 Oct1³ | −1.27 | 0.11 | (0.39) | 0.00 | (0.00) | 0 |
| upstream 2:1 NFkB¹ | −1.26 | 0.09 | (0.30) | 0.05 | (0.22) | 1.54 × 10⁻¹ |
| upstream 100:10 MIR¹ × upstream 100:10 SINE_Alu² | −1.25 | 220.09 | (225.99) | 78.31 | (77.03) | 1.77 × 10⁻¹⁴ |
| upstream 10:5 AluS² × upstream 2:1 BPVE2³ | −1.24 | 2.29 | (4.66) | 0.15 | (0.93) | 0 |
| upstream 5:0 CEBP³ | 1.23 | 0.16 | (0.37) | 0.20 | (0.41) | 2.63 × 10⁻¹ |
| downstream 10:100 LINE_CR1² × upstream 10:0 AP1¹ | −1.22 | 4.29 | (6.65) | 0.96 | (1.61) | 3.33 × 10⁻¹⁶ |
| upstream 100:10 AluS¹ × upstream 3:2 COUP³ | −1.21 | 30.04 | (26.21) | 12.90 | (11.02) | 2.74 × 10⁻¹² |
| upstream 10:9 NFuE5¹ | −1.2 | 0.34 | (0.64) | 0.20 | (0.56) | 6.81 × 10⁻² |
| exon LTR_ERV1² | −1.19 | 0.44 | (5.96) | 0.00 | (0.00) | 0 |
| upstream 10:5 AluS¹ × upstream 2:1 BPVE2³ | −1.18 | 0.83 | (1.71) | 0.05 | (0.32) | 0 |
| downstream 5:10 L1M3² | −1.17 | 0.03 | (0.68) | 0.00 | (0.00) | 2.10 × 10⁻¹² |
| downstream 0:2 AluS¹ × upstream 1:0 BPVE2¹ | −1.16 | 0.27 | (0.84) | 0.00 | (0.00) | 0 |
| upstream 2:0 L1MC¹ | 1.15 | 0.07 | (0.34) | 0.08 | (0.35) | 4.45 × 10⁻¹ |
| downstream 0:1 SINE_MIR¹ × upstream 3:2 AP1¹ | −1.14 | 0.29 | (1.00) | 0.00 | (0.00) | 0 |
| upstream 10:5 SINE_MIR² × upstream 2:0 AluS² | −1.13 | 9.05 | (25.90) | 0.00 | (0.00) | 0 |
| downstream 0:1 SINE_Alu² × upstream 7:6 GTIIC¹ | −1.12 | 3.58 | (13.55) | 0.00 | (0.00) | 0 |
| upstream 1:0 SINE_Alu¹ × upstream 7:6 Oct1³ | −1.11 | 0.16 | (0.47) | 0.00 | (0.00) | 0 |
| upstream 5:0 SINE_Alu² × upstream 8:7 GT2B¹ | −1.1 | 7.19 | (17.39) | 1.40 | (3.55) | 5.11 × 10⁻¹³ |
| downstream 0:5 L1MB² | −1.09 | 0.94 | (4.69) | 0.45 | (1.79) | 4.94 × 10⁻² |
| upstream 100:0 CpGi^{2,10} | −1.08 | 0.18 | (0.15) | 0.17 | (0.12) | 3.12 × 10⁻¹ |
| upstream 100:10 SINE_MIR¹ × upstream 100:0 Alu | −1.07 | 447.93 | (440.90) | 157.03 | (154.06) | 8.10 × 10⁻¹⁵ |
| phase change¹ | | | | | | |
| downstream 10:100 LINE_CR1² × upstream 5:0 AluS¹ | −1.06 | 0.39 | (0.90) | 0.04 | (0.13) | 0 |
| upstream 2:0 AluS² × upstream 5:0 ETFA³ | −1.05 | 2.19 | (7.07) | 0.00 | (0.00) | 0 |
| downstream 10:100 AluY¹ × upstream 100:10 AluS¹ | −1.04 | 291.62 | (389.69) | 97.25 | (119.91) | 7.77 × 10⁻¹³ |
| downstream 10:100 LINE_CR1² × upstream 3:2 TFIID¹ | −1.03 | 0.27 | (0.68) | 0.01 | (0.05) | 0 |
| upstream 5:0 AluS² × m3upIndicator | −1.02 | 6.98 | (10.28) | 1.40 | (3.52) | 1.48 × 10⁻¹² |
| upstream 6:5 GATA1¹ | −1.01 | 0.68 | (0.88) | 0.58 | (0.78) | 2.13 × 10⁻¹ |
| upstream 100:10 L1M3±² | −1 | 50.39 | (302.85) | 96.83 | (305.95) | 1.75 × 10⁻¹ |
| upstream 7:6 Sp1¹ | −0.99 | 0.35 | (1.14) | 0.50 | (0.96) | 1.66 × 10⁻¹ |
| upstream 5:0 SINE_Alu² × upstream 5:0 BPVE2¹ | 0.98 | 43.86 | (59.14) | 12.93 | (19.79) | 2.28 × 10⁻¹² |
| upstream 3:2 Pit1¹ × upstream 10:9 NFuE5³ | −0.97 | 0.17 | (0.63) | 0.00 | (0.00) | 0 |
| upstream 4:3 AR¹ | −0.96 | 12.17 | (4.69) | 10.98 | (4.59) | 5.66 × 10⁻² |
| upstream 2:0 AluS² × upstream 10:9 COUP¹ | −0.95 | 17.48 | (33.29) | 0.30 | (1.91) | 0 |
| downstream 10:100 LINE_CR1² × upstream 5:0 | −0.94 | 3.49 | (7.39) | 0.29 | (0.78) | 0 |
| SINE_Alu² | | | | | | |
| upstream 5:0 SINE_Alu¹ × upstream 5:0 AP1¹ | 0.93 | 33.65 | (40.19) | 10.73 | (15.01) | 4.43 × 10⁻¹² |
| upstream 2:0 AluS±² × m11intronIndicator | −0.92 | 76.80 | (162.84) | 4.68 | (29.57) | 0 |
| downstream 10:100 AluJ² | 0.91 | 3.43 | (2.48) | 2.20 | (1.31) | 3.56 × 10⁻⁷ |
| downstream 5:10 L1MA² | −0.9 | 0.85 | (4.96) | 0.58 | (2.57) | 2.61 × 10⁻¹ |
| upstream 8:7 BPVE2¹ × upstream 2:0 E4TF1³ | −0.89 | 0.09 | (0.36) | 0.00 | (0.00) | 0 |
| upstream 1:0 NFIII³ | 0.88 | 0.70 | (0.46) | 0.65 | (0.48) | 2.43 × 10⁻¹ |
| upstream 10:5 GC² × upstream 100:10 AluS¹ | −0.87 | 1585.80 | (1238.67) | 795.33 | (490.41) | 9.69 × 10⁻¹³ |
| upstream 100:10 AluS² × upstream 3:2 AP1³ | −0.86 | 8.16 | (7.16) | 2.97 | (3.15) | 5.57 × 10⁻¹³ |
| upstream 5:0 LINE_L1² | −0.85 | 7.68 | (13.27) | 5.61 | (10.69) | 1.17 × 10⁻¹ |
| upstream 9:8 CP1¹ | −0.84 | 0.05 | (0.25) | 0.00 | (0.00) | 0 |
| upstream 10:5 L1MA±¹ | −0.83 | 0.00 | (0.49) | −0.05 | (0.45) | 2.34 × 10⁻¹ |
| downstream 10:100 AluS±¹ | −0.82 | −0.08 | (2.12) | 0.46 | (2.72) | 1.10 × 10⁻¹ |
| downstream 10:100 SINE_Alu² × upstream 10:0 AP1¹ | 0.81 | 290.85 | (240.38) | 144.13 | (86.25) | 1.96 × 10⁻¹³ |
| upstream 10:5 FLAM¹ | 0.8 | 0.17 | (0.44) | 0.10 | (0.30) | 8.06 × 10⁻² |
| upstream 5:0 AluS¹ × upstream 9:8 COUP³ | 0.79 | 1.64 | (1.98) | 0.50 | (0.75) | 4.92 × 10⁻¹² |
| downstream 10:100 AluJ² × downstream 5:10 SINE_Alu² | 0.78 | 38.62 | (53.95) | 12.50 | (15.75) | 3.94 × 10⁻¹³ |
| upstream 5:0 SINE_Alu² × upstream 2:0 Sp1³ | 0.77 | 11.27 | (15.08) | 3.61 | (5.46) | 4.35 × 10⁻¹¹ |
| downstream 0:5 AluS¹ × upstream 2:1 BPVE2³ | −0.76 | 0.67 | (1.46) | 0.05 | (0.22) | 0 |
| upstream 100:0 MIR phase change² | −0.75 | 0.41 | (0.16) | 0.37 | (0.26) | 1.74 × 10⁻¹ |
| upstream 2:0 SINE_Alu² × upstream 2:1 AP2¹ | −0.74 | 4.12 | (13.16) | 0.00 | (0.00) | 0 |
| downstream 0:1 MIR¹ × upstream 1:0 AP1³ | −0.73 | 0.09 | (0.33) | 0.00 | (0.00) | 0 |
| upstream 5:0 AluS¹ × upstream 8:7 SIF³ | −0.72 | 0.53 | (1.46) | 0.03 | (0.16) | 0 |
| upstream 10:5 SINE_MIR¹ × upstream 2:0 SINE_Alu² | −0.71 | 11.65 | (29.39) | 0.66 | (2.91) | 0 |
| upstream 5:0 NFuE3¹ | 0.7 | 0.36 | (0.64) | 0.43 | (0.59) | 2.40 × 10⁻¹ |
| upstream 5:0 SINE_Alu² × upstream 9:8 COUP³ | 0.69 | 14.50 | (15.32) | 4.48 | (5.56) | 3.46 × 10⁻¹⁴ |
| intron MIR±² × upstream 90:80 CpGi^{1,11} | −0.68 | 81.39 | (319.80) | 1.38 | (3.50) | 0 |
| downstream 0:5 AluJ¹ × upstream 50:40 CpGi^{1,11} | −0.67 | 1.58 | (3.89) | 0.20 | (0.65) | 1.11 × 10⁻¹⁶ |
| upstream 5:0 DNA_MER1_type±² | −0.66 | 48.94 | (128.50) | 31.56 | (80.74) | 9.38 × 10⁻² |
| upstream 100:10 SINE_Alu¹ × upstream 2:1 BPVE2¹ | −0.65 | 31.40 | (59.03) | 2.78 | (8.41) | 0 |
| downstream 5:10 AluS¹ × upstream 100:10 SINE_Alu¹ | −0.64 | 151.46 | (229.53) | 24.95 | (33.88) | 0 |
| upstream 9:8 AP1³ | 0.63 | 0.84 | (0.37) | 0.80 | (0.41) | 2.92 × 10⁻¹ |
| upstream 5:0 SINE_Alu² × upstream 10:0 COUP¹ | 0.62 | 353.25 | (372.57) | 115.04 | (152.24) | 2.27 × 10⁻¹² |
| upstream 1:0 AluS±² × upstream 2:1 NFIII¹ | −0.61 | 102.61 | (315.31) | 0.00 | (0.00) | 0 |
| downstream 5:10 AluS¹ × downstream 0:1 AluS±² | −0.6 | 180.97 | (537.94) | 0.00 | (0.00) | 0 |
| downstream 10:100 AluS² × upstream 5:0 AluY² | −0.59 | 24.05 | (57.00) | 2.15 | (7.83) | 0 |
| upstream 10:5 AluJ² × upstream 10:5 MIR±² | −0.58 | 118.71 | (369.97) | 8.33 | (51.58) | 0 |
| downstream 0:1 MIR¹ × upstream 2:0 AP1¹ | −0.57 | 0.41 | (1.49) | 0.00 | (0.00) | 0 |
| downstream 10:100 MIR3±² × upstream 2:0 SINE_Alu² | −0.56 | 939.36 | (2652.77) | 66.28 | (285.18) | 0 |
| upstream 100:10 AluS¹ × upstream 6:5 ICSBP³ | −0.55 | 27.43 | (26.55) | 11.55 | (10.88) | 1.55 × 10⁻¹¹ |
| upstream 10:5 CpGi¹ × upstream 5:0 AluS² | 0.54 | 36.71 | (59.08) | 7.97 | (12.59) | 0 |
| upstream 5:0 SINE_Alu² × upstream 100:0 L2 phase | 0.53 | 82.44 | (101.53) | 25.81 | (35.89) | 1.75 × 10⁻¹² |
| change¹ | | | | | | |
| downstream 10:100 FRAM¹ × upstream 2:0 AluJ±² | −0.52 | 73.35 | (256.02) | 0.00 | (0.00) | 0 |
| upstream 1:0 IgPE2¹ | −0.51 | 0.02 | (0.15) | 0.00 | (0.00) | 0 |
| downstream 5:10 AluS² × downstream 0:1 AluS² | −0.5 | 55.81 | (160.17) | 0.00 | (0.00) | 0 |
| downstream 0:5 FLAM±² × upstream 2:0 ICSBP¹ | −0.49 | 43.41 | (149.18) | 0.14 | (0.89) | 0 |
| upstream 8:7 CP1³ | 0.48 | 0.04 | (0.20) | 0.03 | (0.16) | 2.51 × 10⁻¹ |
| upstream 1:0 AluS±² × upstream 6:5 COUP³ | −0.47 | 46.39 | (113.64) | 0.00 | (0.00) | 0 |
| intron SINE_MIR±² × upstream 90:80 CpGi^{1,11} | −0.46 | 77.89 | (320.76) | 1.47 | (3.81) | 0 |
| downstream 0:5 AluS² × upstream 2:1 BPVE2¹ | −0.45 | 4.77 | (11.64) | 0.31 | (1.35) | 0 |
| downstream 0:1 SINE_Alu±¹ | 0.44 | 0.04 | (0.82) | 0.00 | (0.55) | 3.43 × 10⁻¹ |
| upstream 5:0 CpGi² × upstream 1:0 SINE_Alu±² | −0.43 | 7072.20 | (19530.74) | 465.95 | (2091.10) | 0 |
| downstream 5:10 AluS² × upstream 2:1 AR¹ | −0.42 | 57.25 | (66.33) | 18.73 | (26.75) | 2.23 × 10⁻¹¹ |
| upstream 100:10 SINE_Alu² × upstream 2:1 BPVE2³ | −0.41 | 6.19 | (10.16) | 0.75 | (2.32) | 0 |
| downstream 5:10 AluS² × upstream 2:0 AluJ² | −0.4 | 20.65 | (63.27) | 0.00 | (0.00) | 0 |
| upstream 2:0 SINE_Alu² × upstream 10:9 GT2B¹ | −0.39 | 5.98 | (17.47) | 0.26 | (1.62) | 0 |
| downstream 10:100 FLAM¹ × downstream 5:10 AluS² | −0.38 | 19.73 | (33.22) | 3.74 | (7.76) | 3.33 × 10⁻¹⁶ |
| upstream 5:0 AluY² × upstream 2:0 BPVE2¹ | −0.37 | 2.02 | (5.93) | 0.14 | (0.91) | 2.22 × 10⁻¹⁶ |
| upstream 2:0 FAM¹ | −0.36 | 0.00 | (0.06) | 0.00 | (0.00) | 0 |
| downstream 5:10 SINE_Alu² × upstream 10:5 SINE_Alu¹ | 0.35 | 41.12 | (65.94) | 10.41 | (15.68) | 2.00 × 10⁻¹⁵ |
| upstream 100:10 AluJ² × upstream 7:6 AP1¹ | −0.34 | 7.16 | (8.55) | 2.49 | (3.04) | 3.46 × 10⁻¹² |
| upstream 2:0 SINE_Alu¹ × upstream 2:0 BPVE2¹ | 0.33 | 1.16 | (2.44) | 0.18 | (0.50) | 1.55 × 10⁻¹⁵ |
| upstream 10:5 L1MB² | 0.32 | 0.68 | (2.83) | 0.60 | (1.84) | 3.90 × 10⁻¹ |
| downstream 10:100 FLAM² × downstream 5:10 AluS² | −0.31 | 2.38 | (4.01) | 0.47 | (0.97) | 1.55 × 10⁻¹⁵ |
| upstream 100:10 AluS¹ × upstream 5:0 BPVE2¹ | −0.3 | 87.38 | (100.91) | 30.50 | (29.91) | 6.00 × 10⁻¹⁵ |
| downstream 10:100 FAM¹ | −0.29 | 0.26 | (0.56) | 0.13 | (0.33) | 8.12 × 10⁻³ |
| downstream 5:10 SINE_Alu² × upstream 2:0 SINE_Alu² | 0.28 | 152.03 | (285.85) | 21.61 | (74.28) | 6.67 × 10⁻¹⁴ |
| upstream 100:10 AluS² × upstream 2:0 AluJ¹ | −0.27 | 3.39 | (9.25) | 0.19 | (0.73) | 0 |
| upstream 2:0 ATF¹ × upstream 2:0 E4TF1³ | −0.26 | 0.30 | (0.91) | 0.03 | (0.16) | 5.10 × 10⁻¹⁴ |
| upstream 1:0 AluS¹ × upstream 2:0 Sp1³ | −0.25 | 0.14 | (0.41) | 0.00 | (0.00) | 0 |
| upstream 100:10 AluS¹ × upstream 3:2 COUP¹ | −0.24 | 72.30 | (84.53) | 30.48 | (26.66) | 1.99 × 10⁻¹² |
| downstream 0:2 DNA_MER2_type¹ | −0.23 | 0.05 | (0.29) | 0.03 | (0.16) | 1.75 × 10⁻¹ |
| upstream 5:0 SINE_Alu¹ × upstream 4:3 TFIID³ | −0.22 | 2.12 | (2.97) | 0.60 | (1.08) | 4.59 × 10⁻¹¹ |
| upstream 2:0 AluS² × upstream 8:7 SIF³ | −0.21 | 2.32 | (7.64) | 0.00 | (0.00) | 0 |
| downstream 5:10 AluS² × upstream 10:5 SINE_Alu¹ | −0.2 | 25.68 | (43.78) | 3.78 | (6.36) | 0 |
| upstream 5:0 AluS¹ × upstream 10:9 NFuE5³ | −0.19 | 0.61 | (1.49) | 0.05 | (0.22) | 0 |
| downstream 5:10 LTR_ERVL¹ | −0.18 | 0.21 | (0.69) | 0.18 | (0.50) | 3.42 × 10⁻¹ |
| downstream 5:10 FLAM¹ | 0.17 | 0.15 | (0.41) | 0.08 | (0.27) | 3.67 × 10⁻² |
| upstream 2:0 DNA_AcHobo² | −0.16 | 0.11 | (1.32) | 0.00 | (0.00) | 0 |
| downstream 10:100 AluJ² × upstream 5:0 AluS² | 0.15 | 45.82 | (68.17) | 8.98 | (17.67) | 2.22 × 10⁻¹⁶ |
| downstream 0:1 L1MB² | 0.14 | 0.48 | (5.11) | 0.22 | (1.36) | 1.16 × 10⁻¹ |
| downstream 10:100 AluY¹ × upstream 5:0 SINE_Alu¹ | 0.13 | 28.15 | (43.61) | 5.80 | (9.77) | 0 |
| downstream 0:2 SINE_Alu¹ × upstream 2:1 BPVE2¹ | −0.12 | 0.56 | (1.50) | 0.03 | (0.16) | 0 |
| downstream 0:2 L1MB¹ | −0.11 | 0.04 | (0.26) | 0.05 | (0.22) | 3.94 × 10⁻¹ |
| downstream 10:100 LINE_CR1² × upstream 100:10 AluS¹ | 0.1 | 7.26 | (13.49) | 1.45 | (3.33) | 7.67 × 10⁻¹⁴ |
| downstream 5:10 AluS¹ × upstream 1:0 AluS² | −0.09 | 15.56 | (49.12) | 0.00 | (0.00) | 0 |
| upstream 100:10 L1MB¹ × upstream 5:0 AluY¹ | −0.08 | 1.46 | (4.19) | 0.15 | (0.58) | 0 |
| upstream 1:0 AluS² × upstream 10:0 NFuE5¹ | −0.07 | 24.38 | (66.70) | 1.18 | (7.46) | 0 |
| downstream 5:10 AluS² × upstream 1:0 AluS¹ | −0.06 | 1.66 | (5.16) | 0.00 | (0.00) | 0 |
| downstream 5:10 SINE_Alu¹ × upstream 1:0 SINE_Alu² | 0.05 | 42.54 | (106.75) | 3.85 | (16.09) | 0 |
| downstream 10:100 LTR_ERVL±² | 0.04 | 292.21 | (693.57) | 450.10 | (1064.61) | 1.80 × 10⁻¹ |
| intron DNA_Tc2±² | 0.03 | 14.45 | (81.71) | 6.91 | (30.34) | 6.53 × 10⁻² |
| downstream 0:2 Other² | −0.02 | 0.62 | (6.75) | 0.00 | (0.00) | 0 |
| upstream 2:0 SINE_Alu² × upstream 5:0 Sp1³ | −0.01 | 10.60 | (15.55) | 2.05 | (5.41) | 1.71 × 10⁻¹² |
| upstream 5:0 SINE_Alu² × upstream 2:1 COUP¹ | 0.01 | 35.57 | (48.80) | 11.25 | (14.45) | 2.53 × 10⁻¹³ |

Unit is kilobases and it refers to the beginning of the first or the end of the last exon, respectively. Corresponding table for SMLR available upon request. For example, “downstream 10:100” refers to the 90 kb window from 10 kb to 100 kb downstream of the last exon.
¹Number of this feature within the sequence window;
±¹denotes the ratio of repeated elements in the “+” versus the “−” orientation with respect to the gene. It is the negative inverse if there are more elements in the “−” orientation than in the “+” orientation;
² Percentage;
±²Ratio of the percentage of the sequence window covered by repeated elements in ± orientation;
³Indicator for presence of this feature within the sequence window;
⁴Indicator for presence of upstream CTCF consensus-binding site.
⁵Indicator for presence of TGTTTGCAG consensus site;
⁶The phase change happened at one of the following LTR elements: MLT1A0, MLT1B, MSTA, MSTB1, MLT1D, MLT2B4, or MLT1G1;
⁷Indicator for presence of CpG island overlapping the last exon;
⁸Indicator for presence of CpG island overlapping the first exon;
⁹Orientation of motif relative to gene;
¹⁰Methylation prone;
¹¹Methylation resistant;
× indicates pairwise interaction between two variables.

TABLE 5
Relevant Features for Prediction of Parental Preference
by Equbits Classifier

| | | Mean | | |
|---|---|---|---|---|
| | | (standard deviation) | | |
| | | Maternally | Paternally | |
| Feature | Weight | Expressed | Expressed | P |
| downstream 10:100 | 19.57 | −0.71 (1.15) | 1.06 (1.46) | 8.49 × |
| AluJ±¹ × | | | | 10⁻⁵ |
| upstream 1:0 | | | | |
| ATF³ | | | | |
| downstream 10:100 | −18.86 | 0.47 (0.51) | 0 (0) | 3.97 × |
| CpGi³ × up- | | | | 10⁻⁴ |
| stream 3:2 Oct1³ | | | | |
| downstream 10:100 | −17.94 | 4.68 (3.37) | 0.85 (0.99) | 5.19 × |
| CpGi¹ × | | | | 10⁻⁵ |
| upstream | | | | |
| 10:0 GATA1³ | | | | |
| upstream 7:6 | −17.75 | 0.47 (0.51) | 0 (0) | 3.97 × |
| APF³ × | | | | 10⁻⁴ |
| upstream 5:4 | | | | |
| BPVE2³ | | | | |
| upstream | −16.86 | 0.37 (0.50) | 0.05 (0.22) | 8.41 × |
| 4:3 E4F1³ | | | | 10⁻³ |
| downstream 50:90 | 16.49 | 0.21 (0.54) | 2.08 (2.97) | 5.93 × |
| LTR_ERVL±¹ | | | | 10⁻³ |
| upstream 10:9 | −16.42 | 0.68 (0.48) | 0.10 (0.31) | 4.42 × |
| MLTF³ × upstream | | | | 10⁻⁵ |
| 7:6 AP1³ | | | | |
| upstream | −15.92 | 0.42 (0.61) | 0.05 (0.22) | 9.90 × |
| 4:3 E4F1¹ | | | | 10⁻³ |
| downstream | −15.45 | 337.17 (781.15) | 1.84 (1.10) | 3.88 × |
| 10:100 AluS±² | | | | 10⁻² |
| upstream 10:5 | −14.9 | 0.47 (0.61) | 0.10 (0.31) | 1.21 × |
| AluY¹ | | | | 10⁻² |
| upstream 100:90 | −14.88 | 0.18 (0.35) | 0.03 (0.11) | 3.47 × |
| CpGi^{2,10} | | | | 10⁻² |
| upstream 10:5 | −14.69 | 1.43 (1.86) | 0.31 (0.95) | 1.31 × |
| AluY² | | | | 10⁻² |
| downstream 10:100 | −14.57 | 4.68 (3.37) | 1.30 (1.45) | 2.36 × |
| CpGi¹ | | | | 10⁻⁴ |
| downstream 10:100 | 14 | −1.31 (1.80) | 0.38 (2.51) | 1.02 × |
| SINE_MIR±¹ | | | | 10⁻² |
| downstream 10:100 | 13.99 | −0.22 (2.05) | 1.65 (2.90) | 1.29 × |
| LTR_ERVL±¹ | | | | 10⁻² |
| downstream 10:100 | 13.86 | 2.28 (3.47) | 70.32 (153.20) | 3.08 × |
| MIR±² | | | | 10⁻² |
| upstream 6:5 | 13.79 | 0.21 (0.42) | 0.60 (0.68) | 1.90 × |
| GT11C¹ | | | | 10⁻² |
| downstream 10:100 | −13.7 | 67.91 (99.59) | 12.00 (29.01) | 1.42 × |
| MIR3±² | | | | 10⁻² |
| upstream 7:6 | −13.32 | 0.16 (0.37) | 0 (0) | 4.14 × |
| CPI¹ | | | | 10⁻² |
| upstream 100:10 | 13.27 | 0.07 (0.21) | 0.73 (1.28) | 1.73 × |
| L1² | | | | 10⁻² |
| upstream 2:0 | −13.02 | 0.11 (0.32) | −0.25 (0.55) | 9.23 × |
| AluS±¹ | | | | 10⁻³ |
| exon 0.225:0.41 | 12.99 | −0.42 (1.24) | 0.26 (0.81) | 2.62 × |
| nucleosome | | | | |
| potential² | | | | 10⁻² |
| upstream 100:10 | 12.83 | 0 (0) | 0.03 (0.08) | 6.64 × |
| LIM² | | | | 10⁻² |
| upstream 100:10 | 12.73 | 0 (0) | 0.15 (0.37) | 4.14 × |
| LIM¹ | | | | 10⁻² |
| downstream 10:100 | 12.66 | 17.90 (22.58) | 61.57 (37.34) | 5.10 × |
| LINE-L1² × | | | | 10⁻⁵ |
| upstream 5:4 APF¹ | | | | |
| upstream 9:8 | 12.63 | 0 (0) | 0.10 (0.31) | 8.13 × |
| NFuE4¹ | | | | 10⁻² |
| upstream 100:10 | −12.58 | 0.49 (1.74) | −1.07 (2.53) | 1.54 × |
| LTR_ERV1±¹ | | | | 10⁻² |
| upstream 100:10 | 12.3 | 0.32 (0.66) | 1.52 (1.76) | 4.67 × |
| LIM4² | | | | 10⁻³ |
| upstream 6:5 | 12.29 | 0.21 (0.42) | 0.50 (0.51) | 3.05 × |
| GTIIC³ | | | | 10⁻² |
| downstream 10:100 | 12.21 | 0.03 (0.05) | 0.11 (0.17) | 3.29 × |
| LINE_CR1² | | | | 10⁻² |

Unit is kilobases and it refers to the beginning of the first or the end of the last exon, respectively. For example, “downstream 10:100” refers to the 90 kb window from 10 kb to 100 kb downstream of the last exon.
¹Number of this feature within the sequence window; ±¹Denotes the ratio of repeated elements in “+” versus “−” orientation with respect to the gene. It is the negative inverse if there are more elements in the “−” orientation than in the “+” orientation;
²Percentage of the sequence window covered by this feature; ±²Ratio of the percentage of the sequence window covered by repeated elements in ± orientation;
³Indicator for presence of this feature within the sequence window; ¹⁰Methylation prone; × indicates pairwise interaction between two variables.

TABLE 6
High-confidence Imprinted Gene Candidates Predicted in
Human and Mouse

| | | Expression | | |
|---|---|---|---|---|
| Gene | Band | Human | Mouse | Description |
| GFI1 | 1p22 | P | M | Oncogenic growth factor |
| | | | | (Gilks et al., 1993), also |
| | | | | involved in develop- |
| | | | | ment (Moroy, 2005). |
| PRDM16, | 1p36 | P | P | Myeloid leukemia gene |
| MEL1 | | | | (Du et al., 2005). |
| Q96PX6 | 2p16 | P | P | |
| MAG12, | 7q21 | M | M | Specifically expressed |
| ACVRIP, | | | | in human brain and |
| | | | | interacts specifically |
| AIP1 | | | | with atrophin-1 (Wood et |
| | | | | al.,1998). A mutation in |
| | | | | atrophin-1 |
| | | | | causes dentatorubral- |
| | | | | pallidoluysian atrophy |
| | | | | (DRPLA), aprogressive |
| | | | | neurodegenerative |
| | | | | disorder (Li et |
| | | | | al., 1993; Koide et |
| | | | | al., 1994; Nagafuchi |
| | | | | et al., 1994). MAG12 |
| | | | | is also involved in |
| | | | | zebrafish development |
| | | | | (Wright etal., 2004). |
| LY6D | 8q24 | P | M | Expressed in head and |
| | | | | neck squamous |
| | | | | carcinoma |
| | | | | (Kato et al.,1998; |
| | | | | (Brakenhoff et al., 1999). |
| KCNK9 | 8q24 | M | M | K + channel protein |
| | | | | involved in neu- |
| | | | | ron apoptosis and cell |
| | | | | tumorigenesis (Patel |
| | | | | & Lazdunski, 2004). |
| NM_173572 | 10q26 | M | M | |
| NKX6-2 | 10q26 | M | M | Shows tissue-specific |
| | | | | regulation with highest |
| | | | | expression in the |
| | | | | brain [4] and is located |
| | | | | near marker D10S217 |
| | | | | that (Mustanski |
| | | | | et al., 2005) found |
| | | | | was maternally linked to |
| | | | | male sexual orientation. |
| ENSG000 | 11p15 | M | M | Contained within an |
| | | | | intron of LSP1, both |
| 00184682 | | | | in human and in mouse. |
| FOXG1C | 14q12 | P | P | Shows haploin- |
| | | | | sufficiency in a |
| | | | | patient with severe |
| | | | | mental retardation, |
| | | | | brain malformations |
| | | | | and microcephaly |
| | | | | (Shoichet et al., 2005). |
| | | | | Mouse ortholog is |
| | | | | essential for normal |
| | | | | development |
| | | | | of the telencephalon |
| | | | | (Xuan et al.,1995). |
| NM_024598 | 16q13 | M | M | |

Genes predicted to be expressed from the maternal or paternal allele are denoted by M or P, respectively. For brevity, genes previously known to be imprinted are not included.

TABLE 7
Genes Proved or Predicted with High Confidence to be Imprinted Map
to Loci Linked to Various Human Conditions

| Condition | Band | Locus | Coord. | Allele | Linkage/Reference |
|---|---|---|---|---|---|
| Alcoholism | 2p14 | TSC0053926 | 66.5 | p | Linkage to alcoholism (LOD = 1.52) (Liu et al., 2005) |
| | | OTX1 | 63.2 | M | Involved in brain development (Boyl et al., 2001) |
| | 12q24 | D12S1045 | 128.8 | m | Linkage to alcoholism (LOD = 3.17; MOD = 3.68) (Liu et al., |
| | | | | | 2005; Strauch et al., 2005) |
| | | Q9HCM7 | 131.6 | M | |
| | 21q22 | D21S1440 | 38.1 | m | Linkage to alcoholism (MOD = 3.86) (Strauch et al., 2005) |
| | | SIM2 | 37.0 | P | Involved in brain development (Goshu et al., 2004). |
| Alzheimer's | 10q2 | D10S583 | 94.4 | | Linkage to Alzheimer's disease (LOD = 3.3; Bertram et al., |
| | | | | | 2000; Ait-Ghezala et al., 2002) |
| | | Q9H6Z8 | 92.4 | P | |
| | 10q24 | D10S1710 | 102.8 | | Linkage to Alzheimer's disease (LOD = 0.9; Bertram et al., |
| | | | | | 2000) |
| | | LDB1 | 103.8 | M | |
| | 12p13 | D12S1623 | 6.8 | | Linkage to Alzheimer's disease (LOD = 3.15; Mayeux et al., |
| | | | | | 2002) |
| | | RBP5 | 7.2 | P | |
| | 12p11 | D12S1042 | 27.5 | | Linkage to Alzheimer's disease (LOD = 1.43; Mayeux et al., |
| | | | | | 2002) |
| | | ABCC9 | 21.9 | M | |
| | 12q13 | D12S398-D12S1632 | 51.5-54.7 | | Linkage to Alzheimer's disease (LOD = 1.40; Scott et al., 2000) |
| | | HOXC9 | 52.7 | M | |
| | | HOXC4 | 52.7 | M | |
| Asthma/ | 3q21 | D3S3606 | 128.7 | m | Linkage to allergic sensitization (Zall = 4.31; Lee et al., 2000a). |
| Atopy | | FTHFD | 128.3 | M | Mice deficient in this gene show decreased hepatic folate |
| | | | | | levels (Champion et al., 1994) |
| | 14q24 | D14S74 | 77.7 | p | Suggestive linkage to atopy and indications for imprinting |
| | | | | | (MOD = 2.88; Strauch et al., 2001) |
| | | ENSG00000183992 | 80.7 | M | |
| Autism | 1q25 | D1S1677-D1S1589 | 156-172.5 | | Linkage to autism (Bartlett et al., 2005) |
| | | HSPA6 | 159.8 | M | |
| | 7q32 | D7S530-D7S640 | 128.9-132.3 | m | Linkage to autism (MLS = 2.31; Lamb et al., 2005) |
| | | CPA4 | 129.7 | M | Known human imprinted gene (Kayashima et al., 2003) |
| | | MEST | 129.9 | P | Known human imprinted gene (Kobayashi et al., 1997) |
| | | D9S158-D9S905 | 136.3-137.2 | | Linkage to autism (MLS = 1.67; Lamb et al., 2005) |
| | | PHPT1 | 137.0 | M | |
| | | EGFL7 | 136.8 | P | |
| | 10p14 | D10S189 | 6.8 | | Linkage to autism (MLS = 1.15) and schizophrenia (LOD = 3.60; |
| | | | | | Lamb et al., 2005; DeLisi et al., 2002) |
| | | GATA3 | 8.1 | P | Regulates the development of serotoninergic neurons (van |
| | | | | | Doorninck et al., 1999). In 30% of autistic people the most |
| | | | | | frequent dysfunction is the increase of serotonin (Baghdadli |
| | | | | | et al., 2002). Haploinsufficiency for GATA3 was observed in a |
| | | | | | patient with autism and severe mental retardation (Verri et al., |
| | | | | | 2004) |
| | 17q11 | D17S1800 | 26.7 | | Linkage to autism-spectrum disorders (MLS = 2.83) (Yonan et |
| | | | | | al., 2003) |
| | 17q11 | D17S1871, | 21.9, 23.7 | p | Suggestive linkage to ASD and indications for imprinting |
| | | D17S1824 | | | (Bartlett et al., 2005) |
| | | PYY2 | 23.6 | P | |
| Bipolar | 1q41 | D1S549 | 217.7 | m | Linkage to bipolar disorder (MLS = 1.43; Mclnnis et al., 2003) |
| disorder | | PTPN14 | 212.6 | M | |
| | 2q36 | D2S396-D2S206 | 230.4-233.4 | m | Linkage to bipolar disorder (HLOD = 2.20; chon et al., 2001) |
| | | TIGD1 | 233.1 | P | |
| | 8q24 | D8S256 | 134.5 | | Linkage to bipolar disorder (NPL = 3.13; Mclnnis et al., 2003) |
| | | KCNK9 | 140.7 | M† | K+ channel protein involved in neuron apoptosis and cell |
| | | | | | tumorigenesis (Patel and Lazdunski, 2004) |
| | 14q32 | D14S65-D14S78 | 96.7-99.5 | p | Linkage to bipolar disorder (HLOD = 2.47; Cichon et al., 2001) |
| | | DLK1 | 100.3 | P | Known human imprinted gene (Wylie et al., 2000; Kobayashi |
| | | | | | et al., 2000) |
| | | MEG3 | 100.4 | M | Known human imprinted gene (Miyoshi et al., 2000) |
| | | RTL1 | 100.4 | M | Imprinted in mouse (Seitz et al., 2003) and sheep (Charlier et |
| | | | | | al., 2001) |
| Fetal | 14q12 | D14S608 | 27.9 | | Paternal UPD results in fetal malformations (Kurosawa et al., |
| malformation | | | | | 2002) |
| | | FOXG1C | 28.3 | P† | Shows haploinsufficiency in a patient with severe mental |
| | | | | | retardation, brain malformations and microcephaly (Shoichet |
| | | | | | et al., 2005) Mouse ortholog is essential for normal |
| | | | | | development of the telencephalon (Xuan et al., 1995) |
| Male | 10q26 | D10S217 | 129.4 | m | Linkage to male sexual orientation (MLOD = 1.81; Mustanski et |
| homosexuality | | | | | al., 2005) |
| | | C10orf91 | 134.1 | M | |
| | | NKX6-2 | 134.4 | M† | Predominantly expressed in the brain (Lee et al., 2001) |
| | | C10orf93 | 134.6 | M | |
| | | VENTX2 | 134.9 | M | |
| | | Q8N377 | 135.0 | M | |
| | | PAOX | 135.1 | M | |
| Obesity/ | 2q37 | D2S2987 | 242.5 | | Linkage to type 2 diabetes (LOD = 3.19; Einarsdottir et al., |
| Diabetes | | | | | 2006) |
| | 2q37 | GATA23A02- | 235.7-237.7 | m | Linkage to body mass index (BMI) and percentage of fat |
| | | GATA178G09M | | | mass (LOD = 2.23/3.34; Guo et al., 2006) |
| | | Q9Y419 | 239.4 | M | |
| | | MYEOV2 | 240.7 | P | |
| | 3q24 | AAT071 | 151.5 | m | Linkage to BMI (LOD = 1.97; Guo et al., 2006) |
| | | ZIC1 | 148.6 | M | |
| | 19q13 | Mfd232 | 55.6 | m | Linkage to BMI (LOD = 1.81; Guo et al., 2006) |
| | | LILRB4 | 59.9 | M | |
| Schizophrenia | 1q42 | D1S2709 | 228.3 | | Linkage to schizophrenia (LOD = 2.71; Ekelund et al., 2001) |
| | | OBSCN | 224.7 | P | |
| | | HIST3H2BB | 225 | M | |
| | 8p11 | D8S532 | 40.9 | | Linkage to schizophrenia (LOD = 3.06; Stefansson et al., |
| | | | | | 2002) |
| | | PURG | 31 | P | |
| | 9q21 | D9S922 | 80 | | Linkage to schizophrenia (LOD = 1.95; Hovatta et al., 1999) |
| | | NP_001001670 | 81.8 | M | |
| | 22q11 | PRODH2- | 17.3 | | Linkage to schizophrenia (Liu et al., 2002) |
| | | DGCR6 | | | |
| | | DGCR6 | 17.3 | M | Expressed in the developing and adult mouse brain (Maynard |
| | | | | | et al., 2003) |

The table lists loci that have previously been linked to various human conditions, and high-confidence imprinted gene candidates that map into or within 10 Mb (or less) of that locus. If a locus has been observed to have a parent-of-origin effect, this is denoted by a lowercase m or p, for maternal or paternal effects, respectively. Genes predicted to be expressed from the maternal or paternal allele are denoted by M or P, respectively. Genes also predicted to be imprinted in the mouse are marked by †. Alleles that have been proved to be exclusively expressed are underlined.

TABLE 8
IndependentNegativeTest Genes

| | | | |
|---|---|---|---|
| Gene | Band | Expression | Reference |

Stochastic monoallelic expression

| | | | |
|---|---|---|---|
| 1L2 | 4q27 | X | Monoallelically expressed |
| | | | (Hollander et al., 1998). |
| 1L4 | 5q23 | X | Monoallelically expressed (Bix |
| | | | & Locksley, 1998). |
| 1L5 | 5q23 | X | Monoallelically expressed (Kelly |
| | | | & Locksley, 2000). |
| 1L13 | 5q23 | X | Monoallelically expressed (Kelly |
| | | | & Locksley, 2000). |
| OR2AI | 7q35 | X | Monoallelically expressed |
| | | | (Singh et al., 2003). |
| TLR4 | 9q33 | X | Monoallelically expressed |
| | | | (Pereira et al., 2003). |
| SFTPD | 10q22 | X | Heterogeneous allele-specific |
| | | | expression in extrapulmonary |
| | | | tissues (Lin & Floros, 2002). |
| KLRCI | 12p13 | X | Monoallelically expressed |
| | | | (Vance et al., 2002). |
| KLRAI | 12p13 | X | Monoallelically expressed |
| | | | (Tanamachi et al., 2001; |
| | | | Byun et al., 2003). |
| NUBP2 | 16p13 | X | Monoallelically expressed |
| | | | (Sano et al., 2001). |
| 1GFALS | 16p13 | X | Monoallelically expressed |
| | | | (Sano et al., 2001). |
| JSAPI | 16p13 | X | Monoallelically expressed |
| | | | (Sano et al., 2001). |
| OR7A17 | 19p13 | X | Monoallelically expressed |
| | | | (Singh et al., 2003). |

Presumed biallelic expression

| | | | |
|---|---|---|---|
| CD2 | 1p13 | X | Synchronously replicated |
| | | | (Mostoslaysky et al., 2001). |
| APOB | 2p24 | X | Synchronously replicated |
| | | | (Kitsberg et al., 1993). |
| GPD2 | 2q24 | X | No evidence of imprinting (Piras |
| | | | et al., 2000). |
| 1GFBP5 | 2q35 | X | No evidence of imprinting (Piras |
| | | | et al., 2000). |
| RPL23 | 2q36 | X | Biallelically expressed (Greally |
| | | | et al., 1998). |
| Gt(ROSA)26asSor | 3p25 | X | Biallelically expressed (Singh et |
| | | | al., 2003). |
| MSX1 | 4p16 | X | No evidence of imprinting (Blin- |
| | | | Wakkach et al., 2001). |
| GHR | 5p12 | X | Biallelically expressed (Buettner |
| | | | et al., 2004). |
| OSMR | 5p13 | X | Biallelically expressed (Buettner |
| | | | et al., 2004). |
| PRLR | 5p13 | X | Biallelically expressed (Buettner |
| | | | et al., 2004). |
| 1L7R | 5p13 | X | Biallelically expressed (Buettner |
| | | | et al., 2004). |
| NPR3 | 5p13 | X | Biallelically expressed |
| | | | (Buettner et al., 2004). |
| SEMA5A | 5p15 | X | Biallelically expressed |
| | | | (Buettner et al., 2004). |
| CDH10 | 5p14 | X | Biallelically expressed (Buettner |
| | | | et al., 2004). |
| GABRA6 | 5q34 | X | Biallelically expressed |
| | | | (Takahashi & Ko, 1993). |
| SLC22A1 | 6q25 | X | Biallelically expressed |
| | | | (Schweifer et al., 1997). |
| SOD2 | 6q25 | X | Biallelically expressed in mouse |
| | | | (Barlow et al., 1991). |
| TC1I | 6q25 | X | Biallelically expressed in mouse |
| | | | (Barlow et al., 1991). |
| MAS1 | 6q25 | X | Biallelically expressed in mouse |
| | | | (Schweifer et al., 1997; Lyle |
| | | | et al., 2000). |
| PLG | 6q26 | X | Biallelically expressed in mouse |
| | | | (Barlow et al., 1991). |
| COL1A2 | 7q21 | X | Biallelically expressed (Mizuno |
| | | | et al., 2002). |
| ACTB | 7p22 | X | Biallelically expressed (Zhang |
| | | | et al., 1994). |
| ACHE | 7q22 | X | Synchronously replicated |
| | | | (Kitsberg et al., 1993). |
| UBE2H | 7q32 | X | Biallelically expressed (Yamada |
| | | | et al., 2003). |
| MKRN1 | 7q34 | X | No evidence of imprinting |
| | | | (Walter & Paulsen, 2003). |
| SDC2 | 8q22 | X | Biallelically expressed (Buettner |
| | | | et al., 2004). |
| FZD6 | 8q22 | X | Biallelically expressed (Buettner |
| | | | et al., 2004). |
| NOV | 8q24 | X | Biallelically expressed (Buettner |
| | | | et al., 2004). |
| MYC | 8q24 | X | Synchronously replicated |
| | | | (Chess et al., 1994). |
| DOCK8 | 9p24 | X | Biallelically expressed (Lerer et |
| | | | al., 2005). |
| TYRL | 11p11 | X | Synchronously replicated |
| | | | (Singh et al., 2003). |
| PAX6 | 11p13 | X | Biallelically expressed (van |
| | | | Raamsdonk & Tilghman, |
| | | | 2000). |
| GAS2 | 11p14 | X | No evidence of imprinting (Piras |
| | | | et al., 2000). |
| STIM1 | 11p15 | X | Biallelically expressed (Overall |
| | | | et al., 1998). |
| TPH1 | 11p15 | X | Biallelically expressed (Buettner |
| | | | et al., 2004). |
| CARS | 11p15 | X | Biallelically expressed (Clark et |
| | | | al., 2002). |
| RNH | 11p15 | X | Biallelically expressed |
| | | | (Rachmilewitz et al., 1993). |
| TH | 11p15 | X | Biallelically expressed (Reik & |
| | | | Walter, 2001). |
| ASCL2 | 11p15 | X | Imprinted in mouse but not in |
| | | | human (Monk et al., 2006). |
| CTSD | 11p15 | X | Biallelically expressed in human |
| | | | hydatidiform mole, mature |
| | | | teratoma, and normal |
| | | | placenta (Rachmilewitz et al., |
| | | | 1993). |
| RRM1 | 11p15 | X | No evidence of imprinting |
| | | | (Byrne & Smith, 1993). |
| DUSP8 | 11p15 | X | Biallelically expressed |
| | | | (Goldberg et al., 2003). |
| NAP1L4 | 11p15 | X | Biallelic expression in multiple |
| | | | murine fetal and adult tissues |
| | | | (Hu et al., 1996; Paulsen et |
| | | | al., 1998; Umlauf et al., |
| | | | 2004), not imprinted in the |
| | | | human (Monk et al., 2006). |
| PYGM | 11q13 | X | Synchronously replicated |
| | | | (Kitsberg et al., 1993). |
| CD3D | 11q23 | X | Synchronously replicated |
| | | | (Kitsberg et al., 1993). |
| GAPD | 12p13 | X | Biallelically expressed (Paulsen |
| | | | et al., 1998). |
| CD4 | 12p13 | X | Biallelically expressed |
| | | | (Williamson et al., 1995). |
| DCN | 12q21 | X | Imprinted in mouse but not in |
| | | | human (Monk et al., 2006). |
| RB1 | 13q14 | X | Synchronously replicated |
| | | | (Amiel et al., 1999). |
| YY1 | 14q32 | X | Biallelically expressed |
| | | | (Yevtodiyenko et al., 2002). |
| WARS | 14q32 | X | Biallelically expressed |
| | | | (Yevtodiyenko et al., 2002). |
| PPP2R5C | 14q32 | X | Biallelically expressed (Tierling |
| | | | et al., 2006). |
| DNCHC1 | 14q32 | X | Biallelically expressed (Tierling |
| | | | et al., 2006). |
| HERC2 | 15q11 | X | Biallelically expressed (Chai et |
| | | | al., 2003). |
| NDNL2 | 15q13 | X | Biallelically expressed (Chibuk |
| | | | et al., 2001). |
| DUOX1 | 15q21 | X | No evidence of imprinting |
| | | | (Sandell et al., 2003). |
| SLC28A2 | 15q21 | X | No evidence of imprinting |
| | | | (Sandell et al., 2003). |
| DUOX2 | 15q21 | X | No evidence of imprinting |
| | | | (Sandell et al., 2003). |
| SLC30A4 | 15q21 | X | No evidence of imprinting |
| | | | (Sandell et al., 2003). |
| GATM | 15q21 | X | Imprinted in mouse, but not in |
| | | | human (Monk et al., 2006). |
| TP53 | 17p13 | X | Synchronously replicated |
| | | | (Kitsberg et al., 1993). |
| RPL19 | 17q12 | X | Biallelically expressed (Piras et |
| | | | al., 2000). |
| ERBB2 | 17q12 | X | Synchronously replicated |
| | | | (Amiel et al., 1999). |
| APLP1 | 19q13 | X | Biallelically expressed (Buettner |
| | | | et al., 2004). |
| MAG | 19q13 | X | Biallelically expressed (Buettner |
| | | | et al., 2004). |
| SCN1B | 19q13 | X | Biallelically expressed (Buettner |
| | | | et al., 2004). |
| GRIK5 | 19q13 | X | Biallelically expressed (Buettner |
| | | | et al., 2004). |
| APOE | 19q13 | X | Biallelically expressed (Buettner |
| | | | et al., 2004). |
| KCNA7 | 19q13 | X | Biallelically expressed (Buettner |
| | | | et al., 2004). |
| SYT3 | 19q13 | X | Biallelically expressed (Buettner |
| | | | et al., 2004). |
| GRIN2D | 19q13 | X | Biallelically expressed (Buettner |
| | | | et al., 2004). |
| HRC | 19q13 | X | Biallelically expressed (Buettner |
| | | | et al., 2004). |
| KCNC3 | 19q13 | X | Biallelically expressed (Buettner |
| | | | et al., 2004). |
| RRAS | 19q13 | X | Synchronously replicated |
| | | | (Kitsberg et al., 1993). |
| E2F1 | 20q11 | X | Biallelically expressed |
| | | | (Williamson et al., 1995). |
| BC10, BLCAP | 20q11 | X | Biallelically expressed (Evans et |
| | | | al., 2001; John et al., 2001). |
| PLCG1 | 20q12 | X | Biallelically expressed |
| | | | (Williamson et al., 1995). |
| PPGB | 20q13 | X | Biallelically expressed |
| | | | (Williamson et al., 1994). |
| TNFRSF5 | 20q13 | X | Biallelically expressed |
| | | | (Williamson et al., 1995). |
| KCNB1 | 20q13 | X | Biallelically expressed |
| | | | (Williamson et al., 1995). |
| CYP24A1 | 20q13 | X | Biallelically expressed |
| | | | (Williamson et al., 1995). |
| EDN3 | 20q13 | X | Biallelically expressed |
| | | | (Williamson et al., 1995). |
| PCK1 | 20q13 | X | Biallelically expressed |
| | | | (Williamson et al., 1995). |
| NTSR1 | 20q13 | X | Biallelically expressed |
| | | | (Williamson et al., 1995). |
| BMP7 | 20q13 | X | No evidence of imprinting |
| | | | (Marker et al., 1995). |
| CDH4 | 20q13 | X | Synchronously replicated |
| | | | (Williamson et al., 1995). |
| RUNX1 | 21q22 | X | Synchronously replicated |
| | | | (Dotan et al., 2000). |
| PFKL | 21q22 | X | Synchronously replicated |
| | | | (Kitsberg et al., 1993). |

Expression can be one of the following: P (imprinted and paternally expressed), M (imprinted and maternally expressed), or X (not imprinted). All 101 genes were correctly predicted not to be imprinted by the combined classifier.

TABLE 9
Training Genes of Known Imprint Status

| Gene | Band | Expr. | Reference |
|---|---|---|---|
| Imprinted | | | |
| ARHI | 1p31 | P | Yu et al., 1999; Luo et al., 2001 |
| TP73 | 1p36 | M | Kaghad et al., 1997; Mai et al., 1998; Cai |
| | | | et al., 2000 |
| HYMAI | 6q24 | P | Arima et al., 2000 |
| PLAGLI | 6q24 | P | Kamiya et al., 2000 |
| GRBIO | 7p12 | I | Blagitko et al., 2000; Yoshihashi et al., |
| | | | 2000 |
| DLY5 | 7q21 | M | Okita et al., 2003 |
| PPPIR9A | 7q21 | M | Nakabayashi et al., 2004 |
| SGCE | 7q21 | P | Zimprich et al., 2001 |
| PEG10 | 7q21 | P | Ono et al., 2001 |
| CPA4 | 7q32 | M | Kayashima et al., 2003 |
| MEST | 7q32 | p | Kobayashi et al., 1997 |
| WTI | 11p13 | P | Dallosso et al., 2004 |
| 1GF2 | 11p15 | p | Ogawa et al., 1993a; Ohlsson et al., 1993; |
| | | | Rainier et al., 1993 |
| OSBPL5 | 11p15 | M | Higashimoto et al., 2002 |
| SLC22A1L | 11p15 | M | Dao et al., 1998; Cooper et al., 1998 |
| CDKNIC | 11p15 | M | Matsuoka et al., 1996; Chung et al., 1996; |
| | | | Taniguchi et al., 1997 |
| ZNF215 | 11p15 | M | Alders et al., 2000 |
| KCNQIDN | 11p15 | M | Xin et al., 2000 |
| PHLDA2 | 11p15 | M | Qian et al., 1997 |
| SLC22AILS | 11p15 | M | Cooper et al., 1998; Bajaj et al., 2004 |
| KCNQ1 | 11p15 | M | Lee et al., 1997 |
| H19 | 11p15 | M | Rainier et al., 1993 |
| 1GF2AS | 11p15 | p | Okutsu et al., 2000 |
| INS | 11p15 | p | Moore et al., 2001 |
| SMPD1 | 11p15 | M | Simonaro et al., 2006 |
| MEG3 | 14q32 | M | Miyoshi et al., 2000 |
| DLKI | 14q32 | p | Wylie et al., 2000; Kobayashi et al., 2000 |
| SNURF | 15q11 | p | Gray et al., 1999 |
| MKRN3 | 15q11 | p | Driscoll et al., 1992; Glenn et al., 1993; |
| | | | Glenn et al., 1997; Jong et al., 1999 |
| NDN | 15q11 | p | MacDonald & Weyrick, 1997; Jay et al., |
| | | | 1997 |
| MAGEL2 | 15q11 | p | Boccaccio et al., 1999; Lee et al., 2000b |
| 1PIf7 | 15q11 | P | Weyrick et al., 1994 |
| UBE3A | 15q12 | M | Rougeulle et al., 1997; Vu & Hoffman, |
| | | | 1997 |
| ATPI0A | 15q12 | M | Herzing et al., 2001; Meguro et al., 2001 |
| TCEB3C | 18q21 | M | Strichman-Almashanu et al., 2002 |
| Z1M2 | 19q13 | p | Murphy et al., 2001; Van den Veyver et |
| | | | al., 2001 |
| NNAT | 20q11 | p | Evans et al., 2001 |
| NESP55 | 20q13 | M | Hayward et al., 1998 |
| L3MBTL | 20q13 | P | Li et al., 2004 |
| GNASI | 20q13 | p | Liu et al., 2000 |
| Non-imprinted | | | |
| NGFB | 1p13 | X | |
| CDKN2C | 1p32 | X | Cost et al., 1997 |
| COMMD1 | 2pl5 | X | Nabetani et al., 1997 |
| CDKN1A | 6p21 | X | Cost et al., 1997 |
| SERP1NB6 | 6p25 | X | |
| IGF2R | 6q25 | X | Kalscheuer et al., 1993; Ogawa et al., |
| | | | 1993 b; Killian et al., 2001 |
| EGFR | 7p11 | X | Wakeling et al., 1998 |
| COBL | 7p12 | X | Hitchims et al., 2002 |
| DDC | 7p12 | X | Hitchims et al., 2002 |
| IGFBP3 | 7p13 | X | Eggelmann et al., 1999; Wakeling et al., |
| | | | 2000 |
| 1GFBP1 | 7p13 | X | Eggelmann et al., 1999; Wakeling et al., |
| | | | 2000 |
| CPA1 | 7q32 | X | Bentley et al., 2003 |
| HSPC216 | 7q32 | X | Yamada et al., 2003 |
| NRFI | 7q32 | X | Yamada et al., 2003 |
| TSGA14 | 7q32 | X | Yamada et at., 2002 |
| KJAA0265 | 7q32 | X | Yamada et al., 2003 |
| Flj14803 | 7q32 | X | Yamada et al., 2003 |
| CPA2 | 7q32 | X | Bentley et al., 2003 |
| UBE2H | 7q32 | X | Yamada et al., 2003 |
| CNTNAP2 | 7q36 | X | |
| EN2 | 7q36 | X | |
| C90RF39 | 9p22 | X | |
| NOR1/NR4A3 | 9q22 | X | |
| SAMD6 | 9q22 | X | |
| C10orf9 | 10p11 | X | |
| SFMBT2 | 10p14 | X | |
| C100RF28 | 10q24 | X | |
| PHEMX | 11p15 | X | Paulsen et al., 2000; Monk et al., 2006 |
| DRD4 | 11p15 | X | Cichon et al., 1996 |
| MUCDHL | 11p15 | X | Goldberg et al., 2003 |
| MRPL23 | 11p15 | X | Ishihara et al., 1998; Paulsen et al., 2000 |
| CD81 | 11p15 | X | Gabriel et al., 1998; Maecker et al., 1998; |
| | | | Monk et al., 2006 |
| TNNT3 | 11p15 | X | Yuan et al., 1996 |
| HRAS | 11p15 | X | Goldberg et al., 2003 |
| TSSC4 | 11p15 | X | Paulsen et al., 2000 |
| C11ORF2 | 11q13 | X | Zhu et al., 2000 |
| NEU3 | 11q13 | X | |
| CDKN1B | 12p13 | X | Cost et al., 1997 |
| NOVA1 | 14q12 | X | |
| CYFIP1 | 15q11 | X | Chai et al., 2003 |
| NIPA2 | 15q11 | X | Chai et al., 2003 |
| TUBGCP5 | 15q11 | X | Chai et al., 2003 |
| NIPA1 | 15g11 | X | Chai et al., 2003 |
| CI50RF2 | 15q11 | X | Farber et al., 2000 |
| OCA2 | 15q12 | X | Chai et al., 2003 |
| GABRB3 | 15q12 | X | Chai et al., 2003 |
| GABRG3 | 15q12 | X | Chai et al., 2003 |
| CABLES | 18q11 | X | |
| IMPACT | 18q11 | X | Morison et al., 2001 |
| JAG1 | 20p12 | X | |
| TH1L | 20q13 | X | Bonthron et al., 2000 |
| CTSZ | 20q13 | X | Bonthron et al., 2000 |

Expression can be one of the following: P (imprinted and paternally expressed), M (imprinted and maternally expressed), or X (Not imprinted). The GRB10 locus encodes oppositely imprinted transcripts and was excluded from the maternal/paternal model (denoted by I).

TABLE 10
Randomly Chosen Control Genes

| Ensembl ID | Band | |
|---|---|---|
| 065183 (WDR3) | 1p12 | i |
| 092621 (PHGDH) | 1p12 | f |
| 134245 (WNT2B) | 1p13 | t |
| 134253 (TRIM45) | 1p13 | t |
| 007341 (ST7L) | 1p13 | t |
| 116793 (PHTF1) | 1p13 | f |
| 122481 (RWDD3) | 1p21 | t |
| 173146 (Q96CI4) | 1p21 | t |
| 117600 | 1p21 | t |
| (NM_014839) | | |
| 162688 (AGL) | 1p21 | f, i |
| 142951 | 1p21 | f |
| 069702 (TGFBR3) | 1p22 | i |
| 122417 (Q9ULJ1) | 1p22 | t |
| 097096 (Q8NDB8) | 1p22 | i |
| 171488 | 1p22 | t |
| (NM_032270) | | |
| 117174 | 1p22 | f |
| (NM_017953) | | |
| 153898 (MCOLN2) | 1p22 | f |
| 162624 (Q9BYB7) | 1p31 | t |
| 178965 (Q8ND41) | 1p31 | f |
| 079739 (PGM1) | 1p31 | i |
| 117114 (LPHN2) | 1p31 | t |
| 116641 (DOC7) | 1p31 | t |
| 162433 (AK3) | 1p31 | f |
| 185483 (ROR1) | 1p31 | t |
| 162402 (USP24) | 1p32 | t |
| 134744 (Q12764) | 1p32 | i |
| 162398 | 1p32 | i |
| (NM_152607) | | |
| 121310 | 1p32 | i |
| (NM_018281) | | |
| 058804 | 1p32 | i |
| (NM_018087) | | |
| 162384 | 1p32 | i |
| (NM_017887) | | |
| 181150 | 1p32 | i |
| 186857 (Q9HBS7) | 1p32 | f |
| 142973 (CYP4B1) | 1p33 | i |
| 186160 | 1p33 | i |
| (NM_178134) | | |
| 054116 (TRAPPC3) | 1p34 | f |
| 132773 (TOE1) | 1p34 | f |
| 126091 (SIAT6) | 1p34 | i |
| 131238 (PPT1) | 1p34 | t |
| 179178 | 1p34 | t |
| (NM_144626) | | |
| 117400 (MPL) | 1p34 | i |
| 127129 (EDN2) | 1p34 | t |
| 171812 (COL8A2) | 1p34 | f, t |
| 117407 (ARTN) | 1p34 | i |
| 185421 | 1p34 | f |
| 186444 (TMSL1) | 1p34 | f |
| 186973 | 1p34 | f |
| 084628 (TCBA1) | 1p35 | f |
| 175089 (Q9BXE6) | 1p35 | f |
| 168528 | 1p35 | f |
| (NM_178865) | | |
| 134668 | 1p35 | i |
| (NM_144569) | | |
| 121774 | 1p35 | t |
| (KHDRBS1) | | |
| 183615 (YSEC) | 1p35 | f, t |
| 125945 (ZNF436) | 1p36 | f |
| 133226 (SRRM1) | 1p36 | i |
| 173413 (Q9BXE6) | 1p36 | t |
| 179589 (Q8NA34) | 1p36 | t |
| 008130 (PPNK) | 1p36 | f |
| 177799 (O4F3) | 1p36 | t |
| 175262 | 1p36 | t |
| (NM_173507) | | |
| 175087 | 1p36 | t |
| (NM_152835) | | |
| 160072 | 1p36 | f |
| (NM_031921) | | |
| 117701 | 1p36 | t |
| (NM_022078) | | |
| 127054 | 1p36 | f |
| (NM_017871) | | |
| 177000 (MTHFR) | 1p36 | t |
| 008125 (MMP23A) | 1p36 | t |
| 158748 (HTR6) | 1p36 | i |
| 162426 (DNB5) | 1p36 | f |
| 117682 (DHDDS) | 1p36 | f |
| 162438 (CTRC) | 1p36 | i |
| 169504 (CLIC4) | 1p36 | f |
| 171735 (CAMTA1) | 1p36 | f |
| 053371 (AKR7A2) | 1p36 | t |
| 158803 | 1p36 | f |
| 186410 | 1p36 | i |
| 160670 (S100A6) | 1q21 | f |
| 177954 (RPS27) | 1q21 | i |
| 143545 (RAB13) | 1q21 | i |
| 163155 (Q96S90) | 1q21 | t |
| 178527 (Q8N9C2) | 1q21 | t |
| 143615 (O14634) | 1q21 | i |
| 160741 | 1q21 | t |
| (NM_181715) | | |
| 143415 | 1q21 | t |
| (NM_020239) | | |
| 143621 (ILF2) | 1q21 | t |
| 131781 (FMO5) | 1q21 | t |
| 143369 (ECM1) | 1q21 | t |
| 132043 | 1q21 | i |
| 163122 | 1q21 | i |
| 183558 | 1q21 | t |
| (HIST1H2AH) | | |
| 183598 | 1q21 | i |
| (NM_021059) | | |
| 187170 (SPRL4A) | 1q21 | t |
| 187173 | 1q21 | i |
| (NM_178428) | | |
| 187223 (SPRL1A) | 1q21 | f |
| 187428 | 1q21 | i |
| (NM_178353) | | |
| 160753 (RUSC1) | 1q22 | i |
| 163239 | 1q22 | i |
| (NM_182499) | | |
| 160752 (FDPS) | 1q22 | i |
| 160716 (CHRNB2) | 1q22 | f |
| 143595 (AQP10) | 1q22 | i |
| 132704 (SPAP1) | 1q23 | f |
| 162729 (IGSF8) | 1q23 | i |
| 158481 (CD1C) | 1q23 | f |
| 158485 (CD1B) | 1q23 | f |
| 186950 (Q96M18) | 1q23 | f, i, t |
| 120370 | 1q24 | t |
| (NM_152281) | | |
| 000457 | 1q24 | f |
| (NM_020423) | | |
| 178454 | 1q24 | t |
| (NM_018578) | | |
| 143167 (GPA33) | 1q24 | t |
| 094975 (C1orf9) | 1q24 | t |
| 162666 (Y040) | 1q25 | i |
| 116147 (TNR) | 1q25 | i |
| 117586 (TNFSF4) | 1q25 | f |
| 172762 (Q9P1E1) | 1q25 | t |
| 135870 (Q8IVE6) | 1q25 | f |
| 162779 | 1q25 | f, t |
| (NM_182766) | | |
| 162787 | 1q25 | f |
| (NM_181572) | | |
| 135862 (LAMC1) | 1q25 | i |
| 183831 | 1q25 | i |
| 143355 (LHX9) | 1q31 | f |
| 122185 (RPS27) | 1q32 | t |
| 174307 (PHLDA3) | 1q32 | t |
| 174514 | 1q32 | i |
| (NM_181644) | | |
| 162757 | 1q32 | t |
| (NM_152485) | | |
| 158715 | 1q32 | t |
| (NM_033102) | | |
| 117153 | 1q32 | i |
| (NM_021633) | | |
| 077152 | 1q32 | f |
| (NM_014176) | | |
| 117691 | 1q32 | t |
| (NM_013349) | | |
| 117650 (NEK2) | 1q32 | i |
| 118193 (KIF14) | 1q32 | t |
| 162891 (IL20) | 1q32 | f |
| 162809 (Q9NQI1) | 1q41 | t |
| 162814 | 1q41 | f |
| (NM_138796) | | |
| 154309 | 1q41 | i |
| (NM_032890) | | |
| 186063 | 1q41 | i |
| (NM_022831) | | |
| 135763 (Y133) | 1q42 | t |
| 116918 (TSNAX) | 1q42 | t |
| 116957 (TBCE) | 1q42 | f |
| 116991 (Q8NA38) | 1q42 | f |
| 162913 (Q8N372) | 1q42 | t |
| 162885 | 1q42 | i |
| (NM_152490) | | |
| 081692 | 1q42 | i |
| (NM_023007) | | |
| 168148 (HIST3H3) | 1q42 | t |
| 152904 (GGPS1) | 1q42 | f |
| 143669 (CHS1) | 1q42 | t |
| 173576 | 1q42 | f |
| 185495 (Q9H5Q3) | 1q42 | f |
| 116984 (MTR) | 1q43 | i |
| 117009 (KMO) | 1q43 | i |
| 143700 | 1q43 | f, i |
| 182097 (Q96CB2) | 1q43 | t |
| 185346 (Q96B84) | 1q43 | t |
| 179510 (Q9H5F0) | 1q44 | t |
| 162727 (Q96R29) | 1q44 | f |
| 177564 (Q8TC70) | 1q44 | t |
| 177535 (Q8NGW7) | 1q44 | f |
| 171163 | 1q44 | f, i |
| (NM_017865) | | |
| 162711 (CIAS1) | 1q44 | t |
| 185420 (SMYD3) | 1q44 | f |
| 187117 (Q8NG85) | 1q44 | i |
| 178486 (Q8N1I5) | 2p11 | i |
| 068654 (POLR1A) | 2p11 | i |
| 173758 (KV2F) | 2p11 | f |
| 153586 (IGKV4-1) | 2p11 | t |
| 172116 (CD8B1) | 2p11 | i |
| 183281 (PLGL) | 2p11 | t |
| 184943 | 2p11 | i |
| (NM_052871) | | |
| 186854 (Q86V40) | 2p11 | i |
| 115353 (TACR1) | 2p12 | i |
| 163219 (Y053) | 2p13 | i |
| 173027 (WBP1) | 2p13 | i |
| 143977 (SNRPG) | 2p13 | f |
| 075292 | 2p13 | f, t |
| (NM_014497) | | |
| 135638 (EMX1) | 2p13 | i |
| 144048 (DUSP11) | 2p13 | f |
| 114956 (DGUOK) | 2p13 | i |
| 115980 (ANXA4) | 2p13 | i |
| 138072 (Q9NTE1) | 2p14 | t |
| 011523 | 2p14 | i |
| (NM_015147) | | |
| 143995 (MEIS1) | 2p14 | t |
| 014641 (MDH1) | 2p15 | f |
| 152518 (ZFP36L2) | 2p21 | i |
| 152527 (Q8IVE3) | 2p21 | f |
| 138095 (LRPPRC) | 2p21 | t |
| 172877 (Q9BXE6) | 2p22 | t |
| 055332 (PRKR) | 2p22 | t |
| 138068 | 2p22 | i |
| 084733 (RAB10) | 2p23 | t |
| 163795 | 2p23 | f |
| (NM_144631) | | |
| 119777 | 2p23 | t |
| (NM_017727) | | |
| 138028 (CGREF1) | 2p23 | f |
| 186453 (Q96NH8) | 2p23 | i |
| 068697 (LAPTM4A) | 2p24 | f |
| 171863 (RPS7) | 2p25 | f |
| 130508 (Q92626) | 2p25 | i |
| 174685 | 2p25 | i |
| (NM_153011) | | |
| 175652 | 2p25 | f |
| 182717 | 2p25 | f |
| 084090 (STARD7) | 2q11 | t |
| 163126 | 2q11 | f |
| (NM_144994) | | |
| 163699 | 2q11 | i |
| (NM_025024) | | |
| 158435 | 2q11 | i |
| (NM_017546) | | |
| 115446 | 2q11 | i |
| (NM_014044) | | |
| 071073 (MGAT4A) | 2q11 | t |
| 168677 (HMGN1) | 2q11 | t |
| 144191 (CNGA3) | 2q11 | f |
| 115669 (SULT1C1) | 2q12 | t |
| 170417 | 2q12 | f, t |
| (NM_144632) | | |
| 176120 | 2q12 | i |
| (NM_032658) | | |
| 015568 | 2q13 | i |
| (RANBP2L1) | | |
| 179757 (Q9P1E1) | 2q13 | i |
| 175772 | 2q13 | i |
| (NM_152518) | | |
| 153214 | 2q13 | t |
| (NM_032824) | | |
| 136688 (IL1F9) | 2q13 | f |
| 136696 (IL1F8) | 2q13 | i |
| 184764 (RPL22) | 2q13 | i |
| 074054 (CLASP1) | 2q14 | t |
| 176119 (Q96N27) | 2q21 | i |
| 173302 (Q8TDV2) | 2q21 | f |
| 136698 | 2q21 | i |
| (NM_032545) | | |
| 178206 | 2q21 | f |
| (NM_032248) | | |
| 076003 (MCM6) | 2q21 | i |
| 182316 | 2q21 | i |
| 115919 (KYNU) | 2q22 | t |
| 169432 (SCN9A) | 2q24 | i |
| 144285 (SCN1A) | 2q24 | i |
| 174470 (Q96M44) | 2q24 | i |
| 169507 | 2q24 | f |
| (NM_173512) | | |
| 172292 | 2q24 | f |
| 155657 (TTN) | 2q31 | i |
| 172845 (SP3) | 2q31 | i |
| 163364 (Q96D13) | 2q31 | f |
| 175892 (Q8NAT4) | 2q31 | i |
| 128655 (PDE11A) | 2q31 | f |
| 163492 | 2q31 | t |
| (NM_173648) | | |
| 163093 | 2q31 | f |
| (NM_152384) | | |
| 144306 | 2q31 | i |
| (NM_024583) | | |
| 115806 | 2q31 | f |
| (GORASP2) | | |
| 079150 (FKBP7) | 2q31 | f |
| 018510 (AGPS) | 2q31 | t |
| 115942 (ORC2L) | 2q33 | t |
| 155754 | 2q33 | i |
| (NM_152525) | | |
| 155729 | 2q33 | i |
| (NM_152387) | | |
| 178420 | 2q33 | f |
| (NM_030804) | | |
| 115520 | 2q33 | i |
| (NM_025147) | | |
| 155760 (FZD7) | 2q33 | f |
| 155755 (ALS2CR4) | 2q33 | t |
| 186680 | 2q33 | i |
| 168582 (CRYGA) | 2q34 | i |
| 135912 (Y173) | 2q35 | f |
| 127831 (VIL1) | 2q35 | t |
| 135913 (USP37) | 2q35 | f |
| 163526 (TUBA4) | 2q35 | i |
| 115592 (PRKAG3) | 2q35 | t |
| 115655 | 2q35 | i |
| (NM_024085) | | |
| 163497 | 2q35 | t |
| (NM_017521) | | |
| 066216 (TNRC15) | 2q37 | i |
| 176946 (THA4) | 2q37 | i, t |
| 173100 (Q9P0V4) | 2q37 | i |
| 144488 (Q8IVU2) | 2q37 | i |
| 066248 (NGEF) | 2q37 | i |
| 115488 (NEU2) | 2q37 | i |
| 135916 (ITM2C) | 2q37 | i |
| 135930 (EIF4EL3) | 2q37 | i |
| 163286 (ALPPL2) | 2q37 | i |
| 182177 | 2q37 | f |
| 182202 | 2q37 | f |
| 184945 (Q8IXF9) | 2q37 | t |
| 186235 | 2q37 | t |
| 064835 (POU1F1) | 3p11 | t |
| 179021 | 3p11 | f |
| (NM_173824) | | |
| 179799 (Q8NHB5) | 3p12 | f |
| 170837 (GPR27) | 3p13 | t |
| 157467 (O15083) | 3p14 | f |
| 177664 (DNAH12) | 3p14 | i |
| 168374 (ARF4) | 3p14 | f |
| 163638 | 3p14 | f |
| (ADAMTS9) | | |
| 163825 (TMEM7) | 3p21 | t |
| 162244 (RPL29) | 3p21 | t |
| 168237 | 3p21 | t |
| (NM_145262) | | |
| 163817 | 3p21 | i |
| (NM_020208) | | |
| 145029 (NICN1) | 3p21 | t |
| 160808 (MYL3) | 3p21 | i |
| 180929 (GPR62) | 3p21 | i |
| 088538 (DOCK3) | 3p21 | i |
| 121797 (CCRL2) | 3p21 | f |
| 121807 (CCR2) | 3p21 | f |
| 164062 (APEH) | 3p21 | i |
| 184345 (Q8IXL9) | 3p21 | i |
| 186748 | 3p21 | f |
| (NM_018651) | | |
| 163673 (Q9C098) | 3p22 | t |
| 168026 | 3p22 | i |
| (NM_145755) | | |
| 114853 | 3p22 | f |
| (NM_145166) | | |
| 172936 (MYD88) | 3p22 | t |
| 157036 | 3p22 | t |
| (ENDOGL1) | | |
| 185313 (SCN10A) | 3p22 | i |
| 187091 (PLCD1) | 3p22 | i |
| 170266 (GLB1) | 3p23 | t |
| 163513 (TGFBR2) | 3p24 | t |
| 131374 (TBC1D5) | 3p24 | i |
| 185690 (Q9NYD7) | 3p24 | f |
| 186032 | 3p24 | i |
| 171148 (TADA3L) | 3p25 | i |
| 157103 (SLC6A1) | 3p25 | f, i |
| 171135 | 3p25 | t |
| (NM_032492) | | |
| 154743 | 3p25 | t |
| (NM_025265) | | |
| 088726 | 3p25 | f |
| (NM_018306) | | |
| 163703 (CRELD1) | 3p25 | i |
| 131375 (CAPN7) | 3p25 | f |
| 178700 | 3q11 | f |
| (NM_176815) | | |
| 178694 | 3q11 | t |
| (NM_022072) | | |
| 178660 | 3q11 | i |
| 181065 (Q9P161) | 3q13 | f |
| 163428 (Q96CX6) | 3q13 | i |
| 144848 | 3q13 | f |
| (NM_022488) | | |
| 121570 | 3q13 | t |
| (NM_018189) | | |
| 091972 (MOX2) | 3q13 | f |
| 082701 (GSK3B) | 3q13 | f |
| 121594 (CD80) | 3q13 | i |
| 144811 | 3q13 | f |
| 182491 | 3q13 | f |
| 058262 (S612) | 3q21 | i |
| 114544 | 3q21 | i |
| (NM_017836) | | |
| 065534 (MYLK) | 3q21 | i |
| 173702 (MUC13) | 3q21 | t |
| 184621 (Q9HDC0) | 3q21 | i |
| 163785 (RYK) | 3q22 | t |
| 170883 (Q9BXE5) | 3q22 | t |
| 163770 (Q86XG3) | 3q22 | f |
| 163864 (NMNAT3) | 3q23 | i |
| 175110 (MRPS22) | 3q23 | f |
| 114124 (GPRK7) | 3q23 | i |
| 152977 (ZIC1) | 3q24 | i |
| 181467 (RAP2B) | 3q25 | t |
| 174928 | 3q25 | t |
| (NM_173657) | | |
| 144974 | 3q25 | f, i |
| (NM_024621) | | |
| 118855 | 3q25 | f |
| (NM_022736) | | |
| 163659 | 3q25 | i |
| (NM_015508) | | |
| 114771 (AADAC) | 3q25 | f |
| 169760 (NLGN1) | 3q26 | t |
| 136521 (NDUFB5) | 3q26 | i |
| 171109 (MFN1) | 3q26 | f, t |
| 176494 | 3q26 | t |
| 177694 | 3q26 | f |
| 073849 (SIAT1) | 3q27 | t |
| 145012 (LPP) | 3q27 | t |
| 090539 (CHRD) | 3q27 | f |
| 161204 (ABCF3) | 3q27 | t |
| 058705 (IL1RAP) | 3q28 | t |
| 119231 (SEN5) | 3q29 | f |
| 178413 (Q8N266) | 3q29 | i |
| 173950 | 3q29 | i, t |
| (NM_152531) | | |
| 163975 (MFI2) | 3q29 | f |
| 075711 (DLG1) | 3q29 | i |
| 170455 (CNGA1) | 4p12 | i |
| 145246 (ATP10D) | 4p12 | f |
| 170462 | 4p12 | t |
| 183380 | 4p12 | f |
| 163281 | 4p13 | f |
| (NM_138335) | | |
| 174123 (TLR10) | 4p14 | f |
| 154279 (Q8WZ27) | 4p14 | f |
| 121895 | 4p14 | f |
| (NM_024943) | | |
| 174343 (CHRNA9) | 4p14 | t |
| 175524 (Q9UN81) | 4p15 | i |
| 163142 (Q8TE30) | 4p15 | f |
| 053900 | 4p15 | t |
| (NM_013367) | | |
| 159788 (RGS12) | 4p16 | t |
| 177631 | 4p16 | t |
| (NM_182524) | | |
| 130997 | 4p16 | f |
| (NM_181808) | | |
| 178988 | 4p16 | t |
| (NM_152301) | | |
| 179010 | 4p16 | i |
| (NM_033296) | | |
| 159692 (CTBP1) | 4p16 | f, t |
| 087269 (C4orf9) | 4p16 | t |
| 170891 (C17) | 4p16 | f |
| 184855 | 4p16 | t |
| 109184 | 4q11 | i |
| (NM_015115) | | |
| 179378 | 4q13 | i |
| (NM_006692) | | |
| 124882 (EREG) | 4q13 | f |
| 087128 (DES1) | 4q13 | i |
| 124875 (CXCL6) | 4q13 | t |
| 135222 (CSN2) | 4q13 | f |
| 081051 (AFP) | 4q13 | i |
| 079557 (AFM) | 4q13 | i |
| 186942 (Q9BQR7) | 4q13 | i |
| 173130 (Q9P1E1) | 4q21 | t |
| 138670 | 4q21 | t |
| (NM_152545) | | |
| 138759 (FRAS1) | 4q21 | i |
| 138769 (CDKL2) | 4q21 | t |
| 118785 (SPP1) | 4q22 | t |
| 174421 (Q9P1E1) | 4q22 | t |
| 163644 | 4q22 | i |
| (NM_152542) | | |
| 138641 (HERC3) | 4q22 | t |
| 052592 (DMP1) | 4q22 | i |
| 164035 | 4q24 | f |
| (NM_016242) | | |
| 179078 | 4q24 | i |
| 109534 (NOLA1) | 4q25 | t |
| 174720 | 4q25 | f |
| (NM_016648) | | |
| 145384 (FABP2) | 4q26 | t |
| 170917 (NUDT6) | 4q27 | i |
| 138686 (BBS7) | 4q27 | i |
| 164057 | 4q28 | t |
| 109424 (UCP1) | 4q31 | i |
| 153130 (SCOC) | 4q31 | f |
| 137460 (Q9C0D6) | 4q31 | f |
| 151623 (NR3C2) | 4q31 | t |
| 137463 | 4q31 | t |
| (NM_032623) | | |
| 180484 | 4q31 | f |
| (NM_024914) | | |
| 109452 (INPP4B) | 4q31 | i |
| 164142 | 4q31 | f |
| 151005 | 4q32 | f |
| (NM_032136) | | |
| 171557 (FGG) | 4q32 | f |
| 164117 (FBXO8) | 4q34 | i |
| 185075 (Q9H399) | 4q34 | t |
| 173320 (Q9P2F5) | 4q35 | t |
| 180712 | 4q35 | i |
| (NM_173796) | | |
| 164303 | 4q35 | i |
| (NM_153343) | | |
| 109771 | 4q35 | t |
| (NM_018409) | | |
| 168556 (ING1L) | 4q35 | f |
| 151726 (FACL6) | 4q35 | f, t |
| 075705 (DUX2) | 4q35 | t |
| 182552 | 4q35 | t |
| (NM_152682) | | |
| 186158 | 4q35 | i |
| 172239 | 5p12 | t |
| (NM_182789) | | |
| 178846 | 5p13 | f |
| (NM_175921) | | |
| 113361 (CDH6) | 5p13 | f |
| 113460 (BRIX) | 5p13 | i |
| 182564 | 5p13 | t |
| 182977 (Q9P1I1) | 5p13 | t |
| 153416 (ZDHHC11) | 5p15 | t |
| 142319 (SLC6A3) | 5p15 | i |
| 133398 (Q9BTT4) | 5p15 | i |
| 164363 | 5p15 | i |
| (NM_182632) | | |
| 173545 | 5p15 | i |
| (NM_033414) | | |
| 125063 | 5p15 | f |
| (NM_017808) | | |
| 176788 (BASP1) | 5p15 | i |
| 184204 | 5p15 | f |
| 164512 | 5q11 | f |
| (NM_024669) | | |
| 153914 (SFRS12) | 5q12 | f |
| 113598 | 5q12 | i |
| (NM_019072) | | |
| 164253 | 5q13 | i |
| (NM_018268) | | |
| 132835 | 5q13 | t |
| (NM_013303) | | |
| 113161 (HMGCR) | 5q13 | f |
| 181104 (F2R) | 5q13 | f |
| 164300 | 5q14 | f |
| (NM_178276) | | |
| 164299 | 5q14 | i |
| (NM_032567) | | |
| 174715 | 5q14 | f |
| 176819 | 5q14 | t |
| 183772 (CMYA5) | 5q14 | f |
| 133302 | 5q15 | f |
| (NM_032290) | | |
| 185261 | 5q15 | i |
| (NM_173665) | | |
| 169736 (Q9NS32) | 5q21 | f |
| 184213 | 5q21 | f |
| (NM_173488) | | |
| 134982 (APC) | 5q22 | i |
| 125341 (SLC22A5) | 5q23 | f |
| 180831 (Q8N933) | 5q23 | t |
| 164406 (LEA2) | 5q23 | f |
| 138829 (FBN2) | 5q23 | t |
| 182549 | 5q23 | f, i, t |
| 073905 (VDAC1) | 5q31 | t |
| 152700 (SARA2) | 5q31 | t |
| 053108 (Q8TBU0) | 5q31 | t |
| 078795 (PKD2L2) | 5q31 | f |
| 113212 (PCDHB7) | 5q31 | f |
| 113070 (DTR) | 5q31 | i |
| 173250 (Q8TDV0) | 5q32 | f |
| 185777 | 5q32 | f |
| 155846 | 5q33 | t |
| (NM_133263) | | |
| 086570 (FAT2) | 5q33 | i |
| 055163 (CYFIP2) | 5q33 | t |
| 181884 | 5q33 | i |
| 183111 | 5q33 | i |
| 113327 (GABRG2) | 5q34 | i |
| 145864 (GABRB2) | 5q34 | t |
| 113328 (CCNG1) | 5q34 | t |
| 118322 (ATP10B) | 5q34 | i |
| 178187 (ZNF454) | 5q35 | t |
| 170089 (THOC3) | 5q35 | i |
| 131183 (SLC34A1) | 5q35 | t |
| 181538 (Q8N0T8) | 5q35 | f |
| 145912 (NOLA2) | 5q35 | t |
| 170074 | 5q35 | t |
| (NM_173663) | | |
| 168246 | 5q35 | f |
| (NM_152277) | | |
| 146067 | 5q35 | t |
| (NM_019057) | | |
| 040275 | 5q35 | i |
| (NM_017785) | | |
| 064747 | 5q35 | t |
| (NM_015043) | | |
| 169045 (HNRPH1) | 5q35 | i |
| 131459 (GFPT2) | 5q35 | f |
| 160867 (FGFR4) | 5q35 | t |
| 113732 (ATP6V0E) | 5q35 | i |
| 183718 (TRIM52) | 5q35 | t |
| 184550 (Q9H7L9) | 5q35 | f |
| 184714 | 5q35 | i |
| 185005 | 5q35 | t |
| (NM_022471) | | |
| 112210 (RAB23) | 6p11 | f |
| 112200 (ZNF451) | 6p12 | t |
| 065308 (TRAM2) | 6p12 | i |
| 112077 (RHAG) | 6p12 | t |
| 174201 (Q9P1E1) | 6p12 | t |
| 168116 (Q9HCI6) | 6p12 | f |
| 124743 (Q9H511) | 6p12 | i |
| 096087 (GSTA2) | 6p12 | f |
| 146233 (CYP39A1) | 6p12 | i |
| 112715 (VEGF) | 6p21 | i |
| 137394 (TRIM10) | 6p21 | f |
| 175802 (Q9UGE0) | 6p21 | f |
| 168471 (Q9H3W0) | 6p21 | t |
| 178214 (Q96QB7) | 6p21 | t |
| 168379 (Q8WM95) | 6p21 | f |
| 172764 (Q8TDV1) | 6p21 | t |
| 180911 (Q8N925) | 6p21 | i |
| 176415 (Q8N1I6) | 6p21 | f |
| 172738 | 6p21 | t |
| (NM_145316) | | |
| 096080 (MRPS18A) | 6p21 | i |
| 111971 (LY6G5C) | 6p21 | i |
| 096158 (LTB) | 6p21 | f |
| 112095 (HLA-DOA) | 6p21 | t |
| 137333 (DHX16) | 6p21 | t |
| 112195 (C6orf76) | 6p21 | t |
| 007816 (C6orf11) | 6p21 | i |
| 168426 (BTNL2) | 6p21 | f |
| 064999 (ANKS1) | 6p21 | i |
| 124655 (AIF1) | 6p21 | f |
| 137406 | 6p21 | i |
| 161912 | 6p21 | f, t |
| 173580 | 6p21 | t |
| 184729 | 6p21 | t |
| (NM_018540) | | |
| 137403 (HLA-F) | 6p22 | t |
| 178458 | 6p22 | t |
| (HIST1H3A) | | |
| 146047 | 6p22 | f |
| (HIST1H2BA) | | |
| 161777 (HCG9) | 6p22 | i |
| 112293 (GPLD1) | 6p22 | t |
| 137414 (FAM8A1) | 6p22 | i |
| 112242 (E2F3) | 6p22 | i, t |
| 168405 (CMAH) | 6p22 | i |
| 124508 (BTN2A2) | 6p22 | t |
| 183679 (HIST1H4J) | 6p22 | f |
| 185193 (Q9BXE2) | 6p22 | f |
| 185694 | 6p22 | i |
| 112149 (CD83) | 6p23 | f |
| 181590 (Q8NC12) | 6p24 | t |
| 124827 (GCM2) | 6p24 | i |
| 184431 | 6p24 | i |
| 185689 | 6p25 | f |
| 112280 (COL9A1) | 6q13 | t |
| 175596 (Q9P1E1) | 6q14 | t |
| 085382 (HACE1) | 6q16 | f |
| 132429 (POPDC3) | 6q21 | i |
| 177214 | 6q21 | t |
| (NM_173559) | | |
| 123510 (BXDC1) | 6q21 | t |
| 146374 (THSD2) | 6q22 | t |
| 111817 (SART2) | 6q22 | i |
| 152894 (PTPRK) | 6q22 | i |
| 111912 (NCOA7) | 6q22 | f |
| 146376 | 6q22 | f |
| (ARHGAP18) | | |
| 146411 (SLC2A12) | 6q23 | f |
| 154269 (ENPP3) | 6q23 | i |
| 146386 (Q9P0A1) | 6q24 | t |
| 135577 (NMBR) | 6q24 | f |
| 111962 (UST) | 6q25 | t |
| 130338 (TULP4) | 6q25 | f |
| 122335 (SERAC1) | 6q25 | f |
| 180821 (RBM16) | 6q25 | t |
| 120253 (NUP43) | 6q25 | i |
| 049618 | 6q25 | f |
| (NM_175863) | | |
| 120276 | 6q25 | t |
| 174218 | 6q25 | f |
| 185068 (Q9BST5) | 6q25 | i |
| 185345 (PARK2) | 6q26 | t |
| 153471 (TCP10) | 6q27 | i |
| 120436 (GPR31) | 6q27 | t |
| 146731 (CCT6A) | 7p11 | t |
| 154997 | 7p11 | t |
| 180594 (Q96C79) | 7p13 | f |
| 106628 (POLD2) | 7p13 | t |
| 015676 | 7p13 | i |
| (NM_015332) | | |
| 106624 (AEBP1) | 7p13 | f |
| 010270 | 7p14 | t |
| (STARD3NL) | | |
| 164542 (Q8NCT3) | 7p14 | i |
| 181211 (Q8NA17) | 7p14 | t |
| 173862 | 7p14 | i |
| (NM_017937) | | |
| 122641 (INHBA) | 7p14 | t |
| 106105 (GARS) | 7p14 | f |
| 187258 (Q86SP4) | 7p14 | f |
| 176514 (Q9UDC8) | 7p15 | f |
| 174487 (Q9BXE6) | 7p15 | f, t |
| 105928 (DFNA5) | 7p15 | f |
| 153790 (C7orf31) | 7p15 | f |
| 105889 | 7p15 | t |
| 186179 | 7p15 | i |
| 186797 | 7p15 | t |
| 106443 (PHF14) | 7p21 | t |
| 146530 | 7p21 | t |
| (NM_182545) | | |
| 173467 | 7p21 | i |
| (NM_176813) | | |
| 106541 (AGR2) | 7p21 | f |
| 146587 | 7p22 | i |
| (NM_021163) | | |
| 164818 | 7p22 | t |
| (NM_017802) | | |
| 106263 (EIF3S9) | 7p22 | f |
| 169549 | 7p22 | f |
| 174959 | 7p22 | i |
| 175987 | 7p22 | i |
| 179800 | 7p22 | f |
| 187127 (POL1) | 7p22 | t |
| 009950 | 7q11 | i |
| (WBSCR14) | | |
| 135174 (Q9Y4L9) | 7q11 | t |
| 133380 | 7q11 | f |
| (NM_153363) | | |
| 177585 | 7q11 | i |
| 184569 | 7q11 | i |
| 146745 | 7q21 | t |
| (NM_032763) | | |
| 105781 (GRM3) | 7q21 | t |
| 157240 (FZD1) | 7q21 | i |
| 127962 | 7q21 | i |
| 166526 (ZNF3) | 7q22 | t |
| 169899 (Q96MA9) | 7q22 | f |
| 167011 (Q8N8M0) | 7q22 | t |
| 078319 (PMS2L1) | 7q22 | t |
| 146834 | 7q22 | f |
| (NM_019606) | | |
| 021461 (CYP3A43) | 7q22 | t |
| 173685 | 7q22 | t |
| 184414 (IRS3L) | 7q22 | f |
| 185055 | 7q22 | t |
| 128519 (TAS2R16) | 7q31 | f, i |
| 135272 (Q9P1T7) | 7q31 | f |
| 106034 | 7q31 | t |
| (NM_024913) | | |
| 106041 (FAM3C) | 7q31 | i |
| 180324 (CAPZA2) | 7q31 | f |
| 146809 (ASB15) | 7q31 | t |
| 128578 (Q9ULQ0) | 7q32 | t |
| 064419 | 7q32 | i |
| (NM_012470) | | |
| 105875 (Q96AE5) | 7q33 | t |
| 122786 (CALD1) | 7q33 | f |
| 127364 (TAS2R4) | 7q34 | t |
| 171082 (Q8N3Z8) | 7q34 | f |
| 184412 | 7q34 | f |
| 122063 (XRCC2) | 7q36 | t |
| 106615 (RHEB) | 7q36 | f |
| 181652 | 7q36 | f |
| (NM_173681) | | |
| 133574 | 7q36 | i |
| (NM_018326) | | |
| 126870 | 7q36 | t |
| (NM_018051) | | |
| 106560 | 7q36 | t |
| (NM_015660) | | |
| 127360 (IAN4L1) | 7q36 | i |
| 106648 (GALNT15) | 7q36 | t |
| 105993 (DNAJB6) | 7q36 | i |
| 164885 (CDK5) | 7q36 | i |
| 170279 (C7orf33) | 7q36 | t |
| 168172 | 8p11 | i |
| (NM_032410) | | |
| 104371 (DKK4) | 8p11 | f |
| 185900 | 8p11 | i |
| (NM_032237) | | |
| 181329 (O95724) | 8p12 | i |
| 133872 | 8p12 | i |
| (NM_016127) | | |
| 184844 | 8p12 | f |
| 147454 | 8p21 | f |
| (NM_016612) | | |
| 147443 (DOK2) | 8p21 | i |
| 069206 (ADAM7) | 8p21 | t |
| 182406 | 8p21 | f |
| 184661 (CDCA2) | 8p21 | i |
| 181897 | 8p22 | t |
| (NM_018422) | | |
| 156011 | 8p22 | i |
| (NM_015310) | | |
| 154316 (TDH) | 8p23 | f |
| 177405 (Q8NAJ9) | 8p23 | f |
| 154359 | 8p23 | f |
| (NM_152271) | | |
| 147364 (FBXO25) | 8p23 | i |
| 177023 (DEFB104) | 8p23 | f |
| 171060 | 8p23 | t |
| 184608 (C8orf12) | 8p23 | f |
| 186600 (Q9UDD8) | 8p23 | f |
| 082556 (OPRK1) | 8q11 | f |
| 047249 (ATP6V1H) | 8q11 | t |
| 157556 (Q8NHT1) | 8q13 | f |
| 165093 (BTF3L2) | 8q13 | i, t |
| 121039 (RDH10) | 8q21 | i |
| 176731 (Q8N0T1) | 8q21 | f |
| 176206 | 8q21 | i |
| (NM_030970) | | |
| 155099 | 8q21 | f |
| (NM_018710) | | |
| 176623 | 8q21 | i |
| (NM_016033) | | |
| 156170 | 8q22 | i |
| (NM_152416) | | |
| 104324 | 8q22 | t |
| (NM_016134) | | |
| 164949 (GEM) | 8q22 | f |
| 155097 | 8q22 | f |
| (ATP6V1C1) | | |
| 147666 | 8q22 | t |
| 147667 | 8q22 | t |
| 183299 | 8q22 | f |
| 147654 (EBAG9) | 8q23 | i |
| 104415 (WISP1) | 8q24 | t |
| 147804 (SLC39A4) | 8q24 | i |
| 161016 (RPL8) | 8q24 | t |
| 170616 (Q9BRH9) | 8q24 | i |
| 180838 (Q8NAM3) | 8q24 | i |
| 132297 (OC90) | 8q24 | f |
| 167702 | 8q24 | f |
| (NM_145754) | | |
| 153310 | 8q24 | t |
| (NM_016623) | | |
| 147724 | 8q24 | f |
| (NM_015912) | | |
| 147684 (NDUFB9) | 8q24 | i |
| 104419 (NDRG1) | 8q24 | f |
| 172172 (MRPL13) | 8q24 | f |
| 179527 (C8orf17) | 8q24 | t |
| 177205 | 8q24 | f |
| 185582 | 8q24 | f |
| 035445 (UNC13) | 9p13 | t |
| 165006 (UBAP1) | 9p13 | f |
| 107371 (RR40) | 9p13 | i |
| 165012 (Q96GJ8) | 9p13 | t |
| 175768 (Q8N4H5) | 9p13 | f |
| 180810 | 9p13 | i, t |
| (NM_014113) | | |
| 137104 (GALT) | 9p13 | i |
| 122735 (DNAI1) | 9p13 | f |
| 122705 (CLTA) | 9p13 | t |
| 164972 (C9orf24) | 9p13 | f |
| 165269 (AQP7) | 9p13 | f |
| 159712 | 9p13 | f |
| 182355 | 9p13 | i |
| (NM_015667) | | |
| 120247 (IFNA13) | 9p21 | i |
| 147885 (IFNA13) | 9p21 | f |
| 147889 (CDKN2A) | 9p21 | i |
| 186758 (Q8N7I0) | 9p21 | f |
| 186802 (IFNA16) | 9p21 | f, i |
| 147893 | 9p22 | i |
| 155156 | 9p22 | f |
| 147852 (VLDLR) | 9p24 | f |
| 080503 | 9p24 | t |
| (SMARCA2) | | |
| 120158 (RCL1) | 9p24 | t |
| 170777 | 9p24 | i |
| (NM_033516) | | |
| 107020 | 9p24 | t |
| (NM_018465) | | |
| 120210 (INSL6) | 9p24 | t |
| 064218 (DMRT3) | 9p24 | f |
| 183276 | 9p24 | i |
| 183354 (Q8IVE5) | 9p24 | f |
| 178798 (Q8NGA9) | 9q12 | f |
| 170215 (Q8NCQ8) | 9q13 | f |
| 184879 | 9q13 | i |
| 165059 (PRKACG) | 9q21 | f |
| 135045 | 9q21 | f |
| (NM_017998) | | |
| 106782 (CHAC) | 9q21 | f |
| 135049 (AGTPBP1) | 9q21 | t |
| 186632 | 9q21 | f |
| 186747 (Q8N493) | 9q21 | f |
| 136936 (XPA) | 9q22 | f |
| 106809 (OGN) | 9q22 | f |
| 021374 (IARS) | 9q22 | t |
| 158122 | 9q22 | i |
| 185544 | 9q22 | t |
| 106692 (FCMD) | 9q31 | f |
| 186943 (Q8NGS7) | 9q31 | t |
| 187003 (ACTL7A) | 9q31 | f |
| 136868 (SLC31A1) | 9q32 | i |
| 173238 (Q9P1E1) | 9q32 | t |
| 173242 | 9q32 | f |
| 136856 (SLC2A8) | 9q33 | t |
| 119446 | 9q33 | t |
| (NM_033117) | | |
| 136950 | 9q33 | i |
| (NM_030978) | | |
| 136861 | 9q33 | i |
| (CDK5RAP2) | | |
| 160446 (ZDHHC12) | 9q34 | f |
| 165699 (TSC1) | 9q34 | t |
| 119363 (SPTAN1) | 9q34 | f |
| 160271 (RALGDS) | 9q34 | f |
| 107290 | 9q34 | t |
| (NM_015046) | | |
| 125484 (GTF3C4) | 9q34 | t |
| 136877 (FPGS) | 9q34 | t |
| 169583 (CLIC3) | 9q34 | f |
| 148408 | 9q34 | t |
| (CACNA1B) | | |
| 160323 | 9q34 | t |
| (ADAMTS13) | | |
| 159247 | 9q34 | t |
| 177185 | 9q34 | f |
| 186350 (RXRA) | 9q34 | f |
| 187195 | 9q34 | f |
| (NM_030898) | | |
| 136737 (ZNF25) | 10p11 | f |
| 173776 (Q96HT2) | 10p11 | f |
| 177353 (O75029) | 10p11 | f |
| 177291 | 10p11 | f |
| (NM_153368) | | |
| 183621 | 10p11 | f |
| (NM_182755) | | |
| 151025 (Q9ULT3) | 10p12 | f |
| 095739 (NMA) | 10p12 | i |
| 150051 | 10p12 | i |
| (NM_173576) | | |
| 182649 | 10p12 | f |
| 152465 (NMT2) | 10p13 | i |
| 134465 (Q8TE30) | 10p15 | f |
| 180525 (Q8N8Z3) | 10p15 | i |
| 134453 | 10p15 | f |
| (NM_032905) | | |
| 165568 | 10p15 | i |
| (NM_031436) | | |
| 134460 (IL2RA) | 10p15 | t |
| 172619 (Y514) | 10q11 | i |
| 177457 | 10q11 | t |
| (NM_173524) | | |
| 165388 | 10q11 | t |
| (NM_153034) | | |
| 170324 | 10q11 | i |
| (NM_152428) | | |
| 152728 | 10q11 | f |
| (NM_147156) | | |
| 148582 | 10q11 | f |
| 122952 (ZWINT) | 10q21 | t |
| 108064 (TCF6L1) | 10q21 | i |
| 170312 (CDC2) | 10q21 | i |
| 079332 (SARA1) | 10q22 | t |
| 107719 (Q9ULE6) | 10q22 | f |
| 122861 (PLAU) | 10q22 | t |
| 178365 (NUDT13) | 10q22 | f |
| 166220 | 10q22 | i |
| (NM_152710) | | |
| 122359 (ANXA11) | 10q22 | f |
| 182180 (DNAJC9) | 10q22 | t |
| 182523 | 10q22 | t |
| 180850 | 10q23 | i |
| (NM_178512) | | |
| 152778 (IFT5) | 10q23 | i |
| 165678 (GHITM) | 10q23 | t |
| 138180 (C10orf3) | 10q23 | i |
| 148835 (TAF5) | 10q24 | i |
| 095637 (SORBS1) | 10q24 | i |
| 156398 (SFXN2) | 10q24 | f |
| 107816 (Q8N1I9) | 10q24 | f |
| 171160 | 10q24 | f |
| (NM_178832) | | |
| 075826 | 10q24 | t |
| (NM_015490) | | |
| 065613 | 10q24 | f |
| (NM_014720) | | |
| 148820 (LDB1) | 10q24 | i |
| 138136 (LBX1) | 10q24 | t |
| 120053 (GOT1) | 10q24 | t |
| 107831 (FGF8) | 10q24 | t |
| 171311 (CSL4) | 10q24 | i |
| 165851 | 10q24 | f |
| 151553 (Q9HCH2) | 10q25 | f |
| 151884 | 10q25 | i |
| 151893 | 10q26 | f |
| (NM_153810) | | |
| 154490 | 10q26 | t |
| (NM_145235) | | |
| 107902 | 10q26 | t |
| (NM_022126) | | |
| 119979 | 10q26 | f |
| (NM_018472) | | |
| 174755 (ACADSB) | 10q26 | i |
| 176584 | 10q26 | f |
| 186730 (DUX4) | 10q26 | f |
| 176567 (Q8NH49) | 11p11 | f |
| 165905 | 11p11 | t |
| (NM_152312) | | |
| 110492 (MDK) | 11p11 | t |
| 180210 (F2) | 11p11 | i |
| 175104 (TRAF6) | 11p12 | i |
| 110422 (HIPK3) | 11p13 | t |
| 186688 | 11p13 | i |
| (NM_181807) | | |
| 121621 | 11p14 | t |
| (NM_031217) | | |
| 187398 (Q86TE4) | 11p14 | t |
| 134339 (SAA2) | 11p15 | f |
| 133818 (RRAS2) | 11p15 | i |
| 151117 | 11p15 | t |
| (NM_153347) | | |
| 166800 | 11p15 | f |
| (NM_144972) | | |
| 166788 | 11p15 | i |
| (NM_138421) | | |
| 179826 | 11p15 | f |
| (NM_054031) | | |
| 151116 | 11p15 | i |
| (NM_018314) | | |
| 129158 | 11p15 | t |
| (NM_012139) | | |
| 129152 (MYOD1) | 11p15 | f |
| 181939 (Q8NGM1) | 11q11 | t |
| 184741 (Q8NH20) | 11q11 | i, t |
| 186117 (Q8NGL2) | 11q11 | f |
| 149150 (SLC43A1) | 11q12 | i |
| 172685 (Q96PG2) | 11q12 | i |
| 176495 (Q8NGI8) | 11q12 | f |
| 109991 (P2RX3) | 11q12 | t |
| 162222 | 11q12 | t |
| (NM_173810) | | |
| 149532 | 11q12 | i |
| (NM_024811) | | |
| 162194 | 11q12 | t |
| (NM_024099) | | |
| 166930 (MS4A5) | 11q12 | i |
| 149516 (MS4A3) | 11q12 | f |
| 134825 (C11orf10) | 11q12 | i |
| 173101 (SIPA1) | 11q13 | f |
| 173959 (RBM14) | 11q13 | f |
| 175514 (Q8TDT2) | 11q13 | i |
| 179263 (Q8NH65) | 11q13 | t |
| 166439 (Q8NCN4) | 11q13 | f |
| 021300 (PLEKHB1) | 11q13 | t |
| 171631 (P2RY6) | 11q13 | i |
| 175591 (P2RY2) | 11q13 | i |
| 178795 | 11q13 | t |
| (NM_182833) | | |
| 173914 | 11q13 | f |
| (NM_031492) | | |
| 172732 | 11q13 | f |
| (NM_025128) | | |
| 132749 (MTL5) | 11q13 | t |
| 168056 (LTBP3) | 11q13 | f |
| 172638 (EFEMP2) | 11q13 | i |
| 175602 (DIPA) | 11q13 | f |
| 175315 (CST6) | 11q13 | i |
| 175334 (BANF1) | 11q13 | t |
| 176245 | 11q13 | t |
| 184154 | 11q13 | t |
| (NM_145309) | | |
| 186642 (PDE2A) | 11q13 | f |
| 187040 | 11q13 | t |
| 118369 (USP35) | 11q14 | i |
| 137509 (PRCP) | 11q14 | t |
| 172946 (Q9P1E1) | 11q21 | t |
| 150312 | 11q21 | t |
| 137693 (YAP1) | 11q22 | f |
| 110723 (SL2B) | 11q22 | f |
| 166253 (Q96LP0) | 11q22 | i |
| 137692 | 11q22 | i |
| (NM_032299) | | |
| 118113 (MMP8) | 11q22 | t |
| 166648 (DNCH2) | 11q22 | f |
| 176610 | 11q22 | i |
| 187069 | 11q22 | i |
| 036672 (USP2) | 11q23 | i |
| 173524 (Q9BXE6) | 11q23 | t |
| 160613 (PCSK7) | 11q23 | t |
| 154114 | 11q23 | i |
| (NM_152715) | | |
| 095110 | 11q23 | t |
| (NM_152315) | | |
| 137747 | 11q23 | t |
| (NM_032046) | | |
| 110367 (DDX6) | 11q23 | i |
| 167283 (ATP5L) | 11q23 | f |
| 110244 (APOA4) | 11q23 | i |
| 182581 (Q9BXE6) | 11q23 | f |
| 023171 (Q9ULL9) | 11q24 | t |
| 170953 (Q8NGG6) | 11q24 | f, t |
| 176952 (Q8N6I7) | 11q24 | t |
| 165526 | 11q24 | t |
| (NM_032795) | | |
| 149552 | 11q24 | f |
| (NM_024556) | | |
| 110013 | 11q24 | t |
| (NM_018978) | | |
| 120457 (KCNJ5) | 11q24 | t |
| 151704 (KCNJ1) | 11q24 | i |
| 109832 (DDX25) | 11q24 | t |
| 183483 (Q8IZY5) | 11q24 | f |
| 185688 (Q8NH79) | 11q24 | i |
| 187072 | 11q24 | i |
| 175724 | 11q25 | t |
| (NM_152711) | | |
| 177340 | 12p11 | f |
| (NM_024799) | | |
| 123106 | 12p11 | t |
| (NM_018318) | | |
| 110888 (C1QDC1) | 12p11 | f |
| 151490 (PTPRO) | 12p12 | f |
| 067182 | 12p13 | f |
| (TNFRSF1A) | | |
| 121314 (TAS2R8) | 12p13 | i |
| 121379 (TAS2R14) | 12p13 | i |
| 013588 (RAI3) | 12p13 | f |
| 126838 (PZP) | 12p13 | t |
| 173342 (PRB1) | 12p13 | f |
| 173391 (OLR1) | 12p13 | f |
| 172322 | 12p13 | f |
| (NM_138337) | | |
| 111671 | 12p13 | i |
| (NM_032641) | | |
| 171792 | 12p13 | f |
| (NM_031465) | | |
| 126740 | 12p13 | f |
| (NM_007273) | | |
| 121373 (KLRC4) | 12p13 | i |
| 111218 (HRMT1L3) | 12p13 | t |
| 111664 (GNB3) | 12p13 | f |
| 118972 (FGF23) | 12p13 | i |
| 111652 (COPS7A) | 12p13 | t |
| 180574 | 12p13 | t |
| 151229 (SLC2A13) | 12q12 | t |
| 151233 (Q8IXV1) | 12q12 | f |
| 186518 (Q96SJ6) | 12q12 | t |
| 166888 (STAT6) | 12q13 | i |
| 172602 (RHO6) | 12q13 | i |
| 076067 (RBMS2) | 12q13 | t |
| 167566 (Q9HCH0) | 12q13 | i |
| 179962 (Q8NGE6) | 12q13 | t |
| 139645 (Q8NB46) | 12q13 | i |
| 139540 | 12q13 | i |
| (NM_173596) | | |
| 139579 | 12q13 | f |
| (NM_024068) | | |
| 139625 (MAP3K12) | 12q13 | i |
| 170477 (KRT4) | 12q13 | f |
| 135472 (FAIM2) | 12q13 | t |
| 139631 (CSAD) | 12q13 | i |
| 177981 (ASB8) | 12q13 | i |
| 167580 (AQP2) | 12q13 | i |
| 135409 (AMHR2) | 12q13 | f |
| 185389 | 12q13 | f |
| (NM_018507) | | |
| 185971 | 12q13 | i |
| 186897 (Q86Z23) | 12q13 | f |
| 177221 (Q8WYW9) | 12q14 | t |
| 155974 (GRIP1) | 12q14 | t |
| 111537 (IFNG) | 12q15 | f |
| 166225 (FRS2) | 12q15 | f |
| 111596 (CNOT2) | 12q15 | t |
| 185393 (Q9BTS6) | 12q15 | t |
| 185563 | 12q15 | f |
| 165899 | 12q21 | f |
| (NM_173591) | | |
| 139330 (KERA) | 12q21 | i |
| 139292 (GPR49) | 12q21 | f |
| 083782 (DSPG3) | 12q21 | i |
| 180318 (CART1) | 12q21 | f |
| 182127 | 12q21 | t |
| 187111 | 12q21 | f |
| 028203 (VEZA) | 12q22 | f, i |
| 139343 (SNRPF) | 12q23 | i |
| 136051 (Q9NV91) | 12q23 | f |
| 166629 (Q96L24) | 12q23 | f |
| 151136 | 12q23 | i |
| (NM_152322) | | |
| 111670 | 12q23 | i |
| (NM_024312) | | |
| 139420 | 12q23 | f |
| (NM_007076) | | |
| 174600 (CMKLR1) | 12q23 | i |
| 139352 (ASCL1) | 12q23 | f |
| 120868 (APAF1) | 12q23 | f |
| 183395 (PMCH) | 12q23 | i |
| 185046 | 12q23 | t |
| (NM_020140) | | |
| 139370 (SLC15A4) | 12q24 | t |
| 061936 (SFRS8) | 12q24 | t |
| 089232 (SCA2) | 12q24 | t |
| 139697 (SBNO1) | 12q24 | f, i |
| 178043 (Q9HA69) | 12q24 | f |
| 180645 (Q9BUH0) | 12q24 | t |
| 177213 (Q96LP1) | 12q24 | i |
| 139767 (Q96JH4) | 12q24 | i |
| 089159 (PXN) | 12q24 | t |
| 177192 (PUS1) | 12q24 | f |
| 089250 (NOS1) | 12q24 | i |
| 130921 | 12q24 | t |
| (NM_152269) | | |
| 111412 | 12q24 | t |
| (NM_024738) | | |
| 135090 | 12q24 | t |
| (NM_016281) | | |
| 135148 | 12q24 | i |
| (NM_006700) | | |
| 122965 (K682) | 12q24 | t |
| 158104 (HPD) | 12q24 | i |
| 135108 (FBXO21) | 12q24 | i |
| 111249 (CUTL2) | 12q24 | f |
| 111707 | 12q24 | f, t |
| 184967 | 12q24 | f |
| (NM_024078) | | |
| 132950 (ZNF237) | 13q12 | t |
| 023957 (TUBA2) | 13q12 | i |
| 139514 (SLC7A1) | 13q12 | t |
| 150459 (SAP18) | 13q12 | t |
| 180776 (Q8N2S7) | 13q12 | i |
| 121390 (PSPC1) | 13q12 | f, t |
| 122038 (POLR1D) | 13q12 | t |
| 150456 | 13q12 | f |
| (NM_174928) | | |
| 102699 (ADPRTL1) | 13q12 | t |
| 073910 | 13q13 | i |
| (NM_023037) | | |
| 133101 (CCNA1) | 13q13 | t |
| 179630 (U124) | 13q14 | i |
| 180331 (Q8IX95) | 13q14 | t |
| 083635 (NUFIP1) | 13q14 | i |
| 171945 | 13q14 | f |
| (NM_030970) | | |
| 102837 | 13q14 | i |
| (NM_006418) | | |
| 150506 (PCDH20) | 13q21 | i |
| 118946 (PCDH17) | 13q21 | i |
| 005810 | 13q22 | f |
| (NM_015057) | | |
| 165621 (GPR80) | 13q32 | i |
| 134900 (TPP2) | 13q33 | t |
| 139780 | 13q33 | f |
| 139832 (RAB20) | 13q34 | i |
| 134905 | 13q34 | t |
| (NM_024537) | | |
| 139835 (GRTP1) | 13q34 | i |
| 057593 (F7) | 13q34 | t |
| 130177 (CDC16) | 13q34 | f |
| 102606 | 13q34 | i |
| (ARHGEF7) | | |
| 129563 (TVA2) | 14q11 | t |
| 166056 (TCA) | 14q11 | i |
| 169488 (Q8NH41) | 14q11 | i |
| 176281 (Q8NGD3) | 14q11 | i |
| 136315 (Q12762) | 14q11 | t |
| 100813 (ACINUS) | 14q11 | f |
| 182545 | 14q11 | i |
| 185271 | 14q11 | f |
| 092108 (C14orf163) | 14q12 | t |
| 176127 (C14orf128) | 14q12 | t |
| 129518 (C14orf11) | 14q13 | f |
| 185941 | 14q13 | i |
| 092208 (SIP1) | 14q21 | f |
| 165506 (C14orf104) | 14q21 | i |
| 182090 (C14orf25) | 14q21 | i |
| 131959 (Q9H373) | 14q22 | f |
| 131969 (C14orf29) | 14q22 | f |
| 126778 (SIX1) | 14q23 | f |
| 126821 (SGPP1) | 14q23 | t |
| 100612 | 14q23 | f |
| (NM_016029) | | |
| 179008 (C14orf39) | 14q23 | t |
| 184902 | 14q23 | f |
| 140044 | 14q24 | t |
| (NM_130469) | | |
| 133997 (MED6) | 14q24 | t |
| 139985 (ADAM21) | 14q24 | f |
| 072110 (ACTN1) | 14q24 | t |
| 187073 (Q86TS2) | 14q24 | i |
| 165417 (GTF2A1) | 14q31 | f |
| 042088 (TDP1) | 14q32 | i |
| 140090 (SLC24A4) | 14q32 | f, t |
| 178069 (Q8WYT3) | 14q32 | t |
| 166428 | 14q32 | t |
| (NM_138790) | | |
| 165943 (MOAP1) | 14q32 | t |
| 130076 (IGHA1) | 14q32 | f |
| 165521 | 14q32 | f |
| 183940 | 14q32 | i |
| 153684 (Q8NDK0) | 15q13 | i |
| 169926 (KLF13) | 15q13 | t |
| 179938 | 15q13 | i |
| 175779 (Q8NAA6) | 15q14 | t |
| 178351 (Q8N345) | 15q14 | f |
| 159495 (TGM7) | 15q15 | f |
| 103932 | 15q15 | i |
| (NM_015540) | | |
| 128928 (IVD) | 15q15 | t |
| 166947 (EPB42) | 15q15 | i |
| 092529 (CAPN3) | 15q15 | t |
| 179646 (Q9UI57) | 15q21 | f |
| 166262 | 15q21 | i |
| (NM_152647) | | |
| 140274 | 15q21 | f |
| 166466 | 15q21 | i |
| 170236 | 15q21 | i |
| 140416 (TPM2) | 15q22 | t |
| 074621 (SLC24A1) | 15q22 | t |
| 090470 (PDCD7) | 15q22 | i |
| 140368 (PSTPIP1) | 15q24 | f |
| 140367 | 15q24 | i |
| (NM_173469) | | |
| 173546 (CSPG4) | 15q24 | t |
| 169553 (Q8N824) | 15q25 | i |
| 173867 (MRPL46) | 15q25 | t |
| 140607 | 15q25 | i |
| 184206 | 15q25 | t |
| 140545 (SPAG10) | 15q26 | f |
| 140534 | 15q26 | i |
| (NM_152259) | | |
| 131873 (CHSY1) | 15q26 | t |
| 173607 | 15q26 | f |
| 183000 | 15q26 | i |
| 183208 | 15q26 | t |
| 184508 (Q8N4P3) | 15q26 | f |
| 185442 (Q8NBH7) | 15q26 | f |
| 185594 | 15q26 | f, i |
| (NM_173499) | | |
| 185907 | 15q26 | f |
| (NM_018621) | | |
| 186092 | 15q26 | t |
| 180096 (SEPT1) | 16p11 | t |
| 175995 | 16p11 | t |
| (NM_175901) | | |
| 179755 | 16p11 | f |
| (NM_153227) | | |
| 179965 | 16p11 | t |
| (NM_016643) | | |
| 149925 (ALDOA) | 16p11 | f |
| 169861 | 16p11 | t |
| 181601 | 16p11 | f |
| 183604 (Q9H2H6) | 16p11 | i |
| 184110 (EIF3S8) | 16p11 | t |
| 175758 (Y220) | 16p12 | t |
| 169344 (UMOD) | 16p12 | i |
| 047578 (Q8N803) | 16p12 | i |
| 179038 | 16p12 | f |
| (NM_145237) | | |
| 103275 (UBE2I) | 16p13 | i, t |
| 103197 (TSC2) | 16p13 | i |
| 095917 (TPSD1) | 16p13 | f |
| 162009 (SSTR5) | 16p13 | i |
| 162065 (Q9ULP9) | 16p13 | t |
| 171559 (Q96EU1) | 16p13 | i |
| 069651 (NPIP) | 16p13 | f |
| 161998 | 16p13 | t |
| (NM_145294) | | |
| 153060 | 16p13 | f |
| (NM_144674) | | |
| 161995 | 16p13 | i |
| (NM_053284) | | |
| 168101 | 16p13 | i |
| (NM_032349) | | |
| 059122 | 16p13 | i |
| (NM_032296) | | |
| 033011 | 16p13 | i |
| (NM_019109) | | |
| 100726 | 16p13 | t |
| (NM_016111) | | |
| 072864 (NDE1) | 16p13 | f, t |
| 102858 (MGRN1) | 16p13 | f |
| 103313 (MEFV) | 16p13 | t |
| 103222 (ABCC1) | 16p13 | t |
| 103229 (ABAT) | 16p13 | i |
| 166737 | 16p13 | i |
| 184629 (Q8NCX2) | 16p13 | f |
| 069329 (VPS35) | 16q11 | t |
| 129635 (Q9P1B8) | 16q11 | t |
| 103460 (TNRC9) | 16q12 | t |
| 103494 (Q9Y2K8) | 16q12 | t |
| 166152 | 16q12 | f |
| (NM_144602) | | |
| 129636 | 16q12 | i |
| (NM_030790) | | |
| 171208 (NETO2) | 16q12 | f, t |
| 169715 (MT1E) | 16q12 | f |
| 102978 (POLR2C) | 16q13 | f, i |
| 070729 (CNGB1) | 16q13 | i |
| 187185 (Q86VG7) | 16q13 | f |
| 103043 (TAX1BP2) | 16q22 | f |
| 140824 (TAT) | 16q22 | i |
| 157405 (Q96JG3) | 16q22 | f |
| 159708 | 16q22 | i |
| (NM_018296) | | |
| 090857 | 16q22 | f |
| (NM_017990) | | |
| 038358 | 16q22 | i |
| (NM_014329) | | |
| 168625 (HYDIN) | 16q22 | f |
| 090863 (GLG1) | 16q22 | i |
| 135723 (FHOD1) | 16q22 | i |
| 103089 (FAXDC1) | 16q22 | f |
| 103018 (CYM5) | 16q22 | i |
| 141076 (CIRH1A) | 16q22 | i |
| 062038 (CDH3) | 16q22 | i |
| 067955 (CBFB) | 16q22 | t |
| 166454 (Y431) | 16q23 | i |
| 166455 | 16q23 | t |
| (NM_152337) | | |
| 153815 | 16q23 | i, t |
| (NM_030629) | | |
| 140905 (GCSH) | 16q23 | t |
| 168589 (DNCL2B) | 16q23 | f |
| 166522 | 16q23 | f |
| 131149 (Y182) | 16q24 | t |
| 140950 (Q9HCG3) | 16q24 | f |
| 131153 | 16q24 | i |
| (NM_016095) | | |
| 124391 (IL17C) | 16q24 | f |
| 178773 (CPNE7) | 16q24 | f |
| 182376 | 16q24 | f |
| (NM_182605) | | |
| 183967 | 16q24 | f |
| 133030 | 17p11 | t |
| (NM_015134) | | |
| 072210 (ALDH3A2) | 17p11 | f |
| 154050 | 17p11 | t |
| 184185 (KCNJ12) | 17p11 | f |
| 141028 (Q96T59) | 17p12 | i |
| 108445 (O95611) | 17p12 | i |
| 175091 | 17p12 | f |
| 154914 (USP43) | 17p13 | f |
| 132388 (UBE2G1) | 17p13 | t |
| 161955 (TNFSF13) | 17p13 | t |
| 181856 (SLC2A4) | 17p13 | t |
| 141504 (SAT2) | 17p13 | i |
| 161929 (Q96MD0) | 17p13 | t |
| 007168 | 17p13 | t |
| (PAFAH1B1) | | |
| 129235 | 17p13 | i |
| (NM_032731) | | |
| 132376 | 17p13 | i |
| (NM_016532) | | |
| 141503 (M4K6) | 17p13 | f |
| 161958 (FGF11) | 17p13 | i |
| 178999 (AURKB) | 17p13 | i |
| 182335 (Q8TE90) | 17p13 | t |
| 184166 (OR1D2) | 17p13 | t |
| 185530 | 17p13 | f |
| (NM_030970) | | |
| 185561 | 17p13 | f |
| 187071 (GPS2) | 17p13 | f |
| 076604 (TRAF4) | 17q11 | i |
| 141316 (SPACA3) | 17q11 | f |
| 160551 (Q9P2I6) | 17q11 | f |
| 173012 (Q8TCQ8) | 17q11 | i |
| 141298 | 17q11 | f |
| (NM_033389) | | |
| 087095 | 17q11 | t |
| (NM_016231) | | |
| 108651 (HC66) | 17q11 | i |
| 108278 (TRIP3) | 17q12 | f |
| 172660 (TAF15) | 17q12 | t |
| 174111 (SOC6) | 17q12 | f |
| 132142 (ACACA) | 17q12 | i |
| 108270 (AATF) | 17q12 | i |
| 178655 | 17q12 | i |
| 108379 (WNT3) | 17q21 | i |
| 131462 (TUBG1) | 17q21 | f |
| 073861 (TBX21) | 17q21 | t |
| 167941 (SOST) | 17q21 | t |
| 131096 (PYY) | 17q21 | i |
| 108819 (PPP1R9B) | 17q21 | i |
| 141696 (NO55) | 17q21 | f |
| 167914 | 17q21 | i |
| (NM_178171) | | |
| 167105 | 17q21 | i |
| (NM_153229) | | |
| 167159 | 17q21 | f |
| (NM_152466) | | |
| 108825 | 17q21 | f |
| (NM_025267) | | |
| 108800 | 17q21 | f |
| (NM_014897) | | |
| 159224 (GIP) | 17q21 | f |
| 178743 | 17q21 | t |
| 180386 | 17q21 | i |
| 182076 (NBR2) | 17q21 | t |
| 183978 | 17q21 | i |
| (NM_014019) | | |
| 184502 (GAS) | 17q21 | f |
| 185845 (Q8N0T2) | 17q21 | i |
| 186916 | 17q21 | i |
| 178012 (PECAM1) | 17q23 | i |
| 153951 (O4D2) | 17q23 | t |
| 136490 | 17q23 | i |
| (NM_030576) | | |
| 108375 | 17q23 | t |
| (NM_017763) | | |
| 087995 (METTL2) | 17q23 | t |
| 187013 (Q86X59) | 17q23 | i |
| 141331 (HELZ) | 17q24 | t |
| 108878 (CACNG1) | 17q24 | i |
| 182481 (KPNA2) | 17q24 | t |
| 132481 (TRIM47) | 17q25 | i |
| 178932 (Q8N811) | 17q25 | f |
| 178789 | 17q25 | t |
| (NM_174892) | | |
| 173818 | 17q25 | f |
| (NM_173627) | | |
| 167302 | 17q25 | t |
| (NM_144679) | | |
| 125457 | 17q25 | t |
| (NM_020679) | | |
| 141580 | 17q25 | i |
| (NM_019613) | | |
| 109065 | 17q25 | i |
| (NM_015654) | | |
| 125445 (MRPS7) | 17q25 | t |
| 166685 (COG1) | 17q25 | i |
| 141527 (CARD14) | 17q25 | t |
| 167281 | 17q25 | f |
| 184703 (SIRT7) | 17q25 | f |
| 185262 | 17q25 | i |
| (NM_182565) | | |
| 185298 | 17q25 | t |
| 175319 (Q14179) | 18p11 | t |
| 176014 | 18p11 | t |
| (NM_032525) | | |
| 132199 | 18p11 | t |
| (NM_017512) | | |
| 168461 | 18p11 | t |
| 183206 | 18p11 | f |
| 141447 (OSBPL1A) | 18q11 | f, t |
| 158201 (ABHD3) | 18q11 | i |
| 134779 | 18q12 | i |
| (NM_015476) | | |
| 141434 (MEP1B) | 18q12 | i |
| 134765 (DSC1) | 18q12 | t |
| 186412 | 18q12 | f, t |
| 186496 (ZNF396) | 18q12 | f |
| 081916 | 18q21 | i |
| (SERPINB8) | | |
| 179981 | 18q22 | t |
| (SDCCAG33) | | |
| 186411 | 18q23 | i |
| 168892 (ZNF253) | 19p13 | f, t |
| 132010 (ZNF20) | 19p13 | i |
| 150732 (YE73) | 19p13 | f |
| 125735 (TNFSF14) | 19p13 | i |
| 181143 (Q9H8T7) | 19p13 | t |
| 132001 (Q9H0M5) | 19p13 | f, i |
| 141933 (Q96GE2) | 19p13 | t |
| 129933 (Q8N7K4) | 19p13 | i |
| 099817 (POLR2E) | 19p13 | i |
| 130313 (PGLS) | 19p13 | f |
| 104883 (PEX11G) | 19p13 | f |
| 176995 (OR7C1) | 19p13 | f |
| 099308 (O60307) | 19p13 | t |
| 175217 | 19p13 | i |
| (NM_138774) | | |
| 167807 | 19p13 | i |
| (NM_080665) | | |
| 130307 | 19p13 | f |
| (NM_031941) | | |
| 129951 | 19p13 | i |
| (NM_024888) | | |
| 132000 | 19p13 | f |
| (NM_024825) | | |
| 079313 | 19p13 | t |
| (NM_020695) | | |
| 125912 | 19p13 | t |
| (NM_020170) | | |
| 130813 | 19p13 | f |
| (NM_018381) | | |
| 167487 | 19p13 | i |
| (NM_018316) | | |
| 171466 | 19p13 | f |
| (NM_017656) | | |
| 105229 | 19p13 | i |
| (NM_015897) | | |
| 127666 | 19p13 | f |
| (NM_014261) | | |
| 064489 (MEF2B) | 19p13 | i |
| 099617 (EFNA2) | 19p13 | t |
| 123146 (CD97) | 19p13 | f |
| 161082 | 19p13 | f |
| (BRUNOL5) | | |
| 115268 | 19p13 | f |
| 183617 (MRPL54) | 19p13 | i |
| 185113 | 19p13 | f |
| (NM_032281) | | |
| 185293 | 19p13 | t |
| 187365 | 19p13 | t |
| (NM_175910) | | |
| 159905 (ZNF234) | 19q13 | t |
| 018607 (ZNF221) | 19q13 | i |
| 159882 (ZNF155) | 19q13 | t |
| 063244 (U2AF) | 19q13 | i |
| 063176 (SPHK2) | 19q13 | t |
| 160296 | 19q13 | t |
| (SIGLECL1) | | |
| 168995 (SIGLEC7) | 19q13 | i |
| 161681 (SHANK1) | 19q13 | t |
| 180281 (Q8N843) | 19q13 | i |
| 179873 (PYA6) | 19q13 | f |
| 104960 (PTOV1) | 19q13 | f |
| 011485 (PPP5C) | 19q13 | f |
| 105568 (PPP2R1A) | 19q13 | t |
| 087074 | 19q13 | f |
| (PPP1R15A) | | |
| 105223 (PLD3) | 19q13 | t |
| 104967 (NOVA2) | 19q13 | f |
| 179932 | 19q13 | f |
| (NM_178511) | | |
| 176472 | 19q13 | i |
| (NM_174945) | | |
| 161652 | 19q13 | f, t |
| (NM_152358) | | |
| 104892 | 19q13 | i |
| (NM_145275) | | |
| 142544 | 19q13 | i |
| (NM_145232) | | |
| 105479 | 19q13 | t |
| (NM_144577) | | |
| 160410 | 19q13 | i |
| (NM_138392) | | |
| 126249 | 19q13 | f |
| (NM_032346) | | |
| 104865 | 19q13 | i |
| (NM_018111) | | |
| 076650 | 19q13 | t |
| (NM_018025) | | |
| 160505 (NAL4) | 19q13 | t |
| 174562 (KLKF) | 19q13 | i |
| 167749 (KLK4) | 19q13 | t |
| 105063 (KB15) | 19q13 | f |
| 167644 (IMUP) | 19q13 | t |
| 160007 (GRLF1) | 19q13 | i |
| 126262 (GPR43) | 19q13 | t |
| 105220 (GPI) | 19q13 | i |
| 123859 (FPRL2) | 19q13 | t |
| 104884 (ERCC2) | 19q13 | f |
| 142025 (DMRTC2) | 19q13 | f |
| 105205 (CLC) | 19q13 | i |
| 170956 | 19q13 | i |
| (CEACAM3) | | |
| 142273 (CBLC) | 19q13 | t |
| 008364 (AP2A1) | 19q13 | t |
| 142513 (ACPT) | 19q13 | t |
| 176898 | 19q13 | f |
| 179930 | 19q13 | t |
| 182582 (Q96GE3) | 19q13 | f |
| 186888 | 19q13 | f |
| 187092 (Q8N0S4) | 19q13 | i |
| 187116 | 19q13 | i |
| (NM_181879) | | |
| 187356 | 19q13 | f |
| 177587 (Q96MG3) | 20p11 | t |
| 179447 (Q8N7Z9) | 20p11 | t |
| 132661 (NXT1) | 20p11 | i, t |
| 101004 | 20p11 | i |
| (NM_025176) | | |
| 173404 (INSM1) | 20p11 | t |
| 101435 (CST9L) | 20p11 | i |
| 125815 (CST8) | 20p11 | i |
| 077984 (CST7) | 20p11 | i |
| 125872 (C20orf75) | 20p12 | f |
| 101247 (C20orf7) | 20p12 | i |
| 172296 (C20orf38) | 20p12 | f |
| 089177 (C20orf23) | 20p12 | f |
| 089123 (C20orf13) | 20p12 | f |
| 132623 (ANKRD5) | 20p12 | i |
| 149497 (Q9BYW8) | 20p13 | i |
| 171864 (PRND) | 20p13 | i |
| 125787 (GNRH2) | 20p13 | t |
| 125903 (DEFB129) | 20p13 | t |
| 125843 (C20orf29) | 20p13 | f, t |
| 149451 (ADAM33) | 20p13 | t |
| 183994 (Q9Y2V8) | 20p13 | f |
| 101400 (SNTA1) | 20q11 | t |
| 125991 | 20q11 | i |
| (SDBCAG84) | | |
| 088303 (Q9NQF5) | 20q11 | i |
| 101464 (CDC91L1) | 20q11 | f |
| 149611 (C20orf93) | 20q11 | t |
| 167104 (BPIL3) | 20q11 | t |
| 182171 | 20q11 | i |
| 183566 | 20q11 | t |
| (NM_173859) | | |
| 171940 (ZNF217) | 20q13 | t |
| 064205 (WISP2) | 20q13 | i |
| 180305 | 20q13 | t |
| (WFDC10A) | | |
| 101150 (TPD52L2) | 20q13 | i |
| 101448 (SPINLW1) | 20q13 | f |
| 124216 (SNAI1) | 20q13 | f |
| 174334 (Q9H3Z8) | 20q13 | t |
| 177410 (Q8N5E3) | 20q13 | f |
| 168734 (PKIG) | 20q13 | t |
| 132786 (O43713) | 20q13 | f |
| 149657 | 20q13 | t |
| (NM_144703) | | |
| 124217 (MOCS3) | 20q13 | f |
| 101052 (C20orf9) | 20q13 | i |
| 132823 (C20orf111) | 20q13 | f |
| 130706 (ADRM1) | 20q13 | i |
| 184402 (SS18L1) | 20q13 | i |
| 155282 | 21q11 | f |
| 185272 (RBM11) | 21q11 | i |
| 156253 (C21orf6) | 21q21 | f |
| 182598 | 21q21 | f |
| 160305 (DIP2) | 21q22 | f |
| 159055 (C21orf45) | 21q22 | i |
| 182871 (COL18A1) | 21q22 | i |
| 184724 | 21q22 | t |
| (KRTAP6-1) | | |
| 184809 (C21orf88) | 21q22 | i |
| 184836 (C21orf86) | 21q22 | f |
| 184900 (SMT3H1) | 21q22 | t |
| 185225 (C21orf32) | 21q22 | t |
| 185397 (C21orf51) | 21q22 | t |
| 185706 (Q8TCY0) | 21q22 | i |
| 187026 | 21q22 | t |
| (KRTAP21-2) | | |
| 128218 (VPREB3) | 22q11 | i |
| 138842 (Q9BWW2) | 22q11 | f |
| 133525 (Q99919) | 22q11 | t |
| 099958 (Q96Q80) | 22q11 | f |
| 178026 (Q8N0S9) | 22q11 | i, t |
| 100034 (PPM1F) | 22q11 | i |
| 100023 (PPIL2) | 22q11 | t |
| 161149 | 22q11 | f |
| (NM_145042) | | |
| 177663 (IL17R) | 22q11 | f |
| 100056 (DGCR14) | 22q11 | t |
| 159664 | 22q11 | i |
| 172963 | 22q11 | t |
| 172981 | 22q11 | i |
| 183229 | 22q11 | f |
| 183307 (CECR6) | 22q11 | i |
| 183506 (Q8WUK7) | 22q11 | f, i |
| 183785 (PEX26) | 22q11 | t |
| 184273 | 22q11 | i |
| 099995 (SF3A1) | 22q12 | i, t |
| 099985 (OSM) | 22q12 | f |
| 100350 | 22q12 | i |
| (NM_024955) | | |
| 100365 (NCF4) | 22q12 | t |
| 100385 (IL2RB) | 22q12 | f |
| 100118 (HMG1L10) | 22q12 | f |
| 128284 (APOL3) | 22q12 | t |
| 175329 | 22q12 | i |
| 182763 (Q96EQ7) | 22q12 | t |
| 183579 (Q9ULT6) | 22q12 | f |
| 184117 | 22q12 | f |
| (NIPSNAP1) | | |
| 184122 (Q96NJ4) | 22q12 | i |
| 184654 (Q8N9L7) | 22q12 | t |
| 100426 (ZBED4) | 22q13 | f, t |
| 100106 (TARA) | 22q13 | f |
| 100241 (SBF1) | 22q13 | t |
| 100413 (RPC8) | 22q13 | f |
| 073150 (PANX2) | 22q13 | t |
| 100266 (PACSIN2) | 22q13 | i |
| 176177 | 22q13 | i, t |
| (NM_152512) | | |
| 100101 | 22q13 | i |
| (NM_024313) | | |
| 128285 (GPR24) | 22q13 | i |
| 184472 (YV02) | 22q13 | f |
| 185022 (MAFF) | 22q13 | i |

Genes used for the calculation of the pairwise interactions, feature selection, and model training are denoted by i, f, and t, respectively. To enhance legibility, the common prefix “ENSG00000” has been dropped from the Ensembl ID. Also listed are gene names and/or GENBANK ® Accession Nos. where applicable.

TABLE 11
Primers Used for Expression Analysis of
DLGAP2and KCNK9

| Name | Sequence 5′- . . . -3′ | Position |
|---|---|---|
| DLGAP2- | ACATGAGAAGCTGGGCACTC | 2585-2604† |
| RT1 | (SEQ ID NO: 3) | |
| DLGAP2- | CGTCACCTCCATCGACTTCT | 2651-2670‡ |
| RT2 | (SEQ ID NO: 4) | |
| DLGAP2- | GGCCGTTTCCACCTGAATC | 2048-2066† |
| M1R | (SEQ ID NO: 5) | |
| DLGAP2- | TGATGCTCTGGGAATTCAG | 2059-2077‡ |
| M2R | (SEQ ID NO: 6) | |
| DLGAP2- | CAGCTACCTTCGAGCCATTC | 1605-1624† |
| M1F | (SEQ ID NO: 7) | |
| DLGAP2 | TAGGCTAGACGTCCAGGAACA | 1603779-1603799 |
| 1F | (SEQ ID NO: 8) | |
| DLGAP2- | TATTGGCAGGACTGAGTGGAG | 1604304-1604284 |
| 1R | (SEQ ID NO: 9) | |
| KCNK9- | CAAGGCCTTCTGCATGTTCT | 53849487-53849468 |
| 1F | (SEQ ID NO: 10) | |
| KCNK9- | GTGAATGACCATGCTGTTGC | 53848983-53849002 |
| 1R | (SEQ ID NO: 11) | |
| KCNK9- | TCCTTCTACTTTGCGATCACG | 53933168-53933148 |
| M1F | (SEQ ID NO: 12) | |
| KCNK9- | CATGGTCAAGAACCTGAGGAC | 53849058-53849078 |
| M1R | (SEQ ID NO: 13) | |

Positions for DLGAP2 primers refer to gi: 37552484 (see also GENBANK® Accession No. NT_023736), chr. 8.27.24 (†), and chr. 8.27.26 (‡). Positions for KCNK9 primers are given for gi: 51467074 (see also GENBANK® Accession No. NT_008046.

### REFERENCES

All references listed in the instant disclosure, including but not limited to all patents, patent applications and publications thereof, scientific journal articles, and database entries (including but not limited to GENBANK®, Ensembl, and dbSNP database entries and all annotations available therein) are incorporated herein by reference in their entireties to the extent that they supplement, explain, provide a background for, or teach methodology, techniques, and/or compositions employed herein.
Alders et al. (2000) Am J Hum Genet 66:1473-1484.
Allen et al. (2003) Proc Natl Acad Sci USA 100:9940-9945.
Altschul et al. (1990) 215 J Mol Biol 403-410.
Amiel et al. (1999) Eur J Hum Genet 7:223-230.
Arima et al. (2000) Genomics 67:248-255.
Arsenian et al. (1998) EMBO J 17:6289-6299.
Ausubel et al. (2002) Short Protocols in Molecular Biology, Fifth ed. Wiley, New York, N.Y., United States of America.
Ausubel et al. (2003) Current Protocols in Molecular Biology, John Wylie & Sons, Inc, New York, N.Y., United States of America.
Baghdadli et al. (2002) Encephale 28:248.
Bajaj et al. (2004) BMC Genet 5:13.
Bantignies & Cavalli (2006) Curr Opin Cell Biol 18:275-283.
Barlow (1993) Science 260:309-310.
Barlow et al. (1991) Nature 349:84-87.
Bartlett et al. (2005) Am J Hum Genet 76, 688.
Batzer et al. (1991) 19 Nucleic Acid Res 5081.
Bentley et al. (2003) J Med Genet 40:249-256.
Bertram et al. (2000) Science 290:2302.
Bix & Locksley (1998) Science 281:1352-1354.
Blagitko et al. (2000) Hum Mol Genet 9:1587-1595.
Blin-Wakkach et al. (2001) Proc Natl Acad Sci USA 98:7336-7341.
Boccaccio et al. (1999) Hum Mol Genet 8:2497-2505.
Bonthron et al. (2000) Hum Genet 107:165-175.
Boyl et al. (2001) Int J Dev Neurosci 19:353.
Brakenhoff et al. (1999) Clin Cancer Res 5:725.
Brandeis at al. (1994) Nature 371:435-438.
Buettner at al. (2004) Mamm Genome 15:199-209.
Byrne & Smith (1993) Hum Genet 93:275-277.
Byun et al. (2003) Intl J Cancer 104:318-327.
Cai at al. (2000) Carcinogenesis 21:683-689.
Chai at al. (2003) Am J Hum Genet 73:898-925.
Champion et al., Proc Natl Acad Sci USA 91, 11338 (1994).
Charlier et al. (2001) Genome Res 11:850-862.
Chess at al. (1994) Cell 78:823-834.
Chibuk et al. (2001) BMC Genet 2.
Chung et al. (1996) Hum Mol Genet 5:1101-1108.
Cichon et al. (1996) Am J Med Genet 67:229-231.
Cichon et al. (2001) Hum Mol Genet 10:2933.
Clark et al. (2002) BMC Genet 11.
Cooper et al. (1998) Genomics 49:38-51.
Cost et al. (1997) Cancer Res 57:926-929.
Dallosso et al. (2004) Hum Mol Genet 13:405-415.
Dao et al. (1998) Hum Mol Genet 7:597-608.
DeLisi at al. (2002) Am J Psychiatry 159:803.
Dotan et al. (2000) Genes Chromosomes Cancer 27:270-277.
Driscoll et al. (1992) Genomics 13:917-924.
Du et al. (2005) Blood 106:3932-3939.
Eggermann et al. (1999) Ann Genet 42:117-121.
Einarsdottir et al., Diabetes 55, 1879 (2006).
Ekelund et al., Hum Mol Genet 10, 1611 (2001).
Eun Kwon et al. (2004) Ann NY Acad Sci 1034:1-18.
Evans et al. (2001) Genomics 77:99-104.
Farber et al. (2000) Genomics 65:174-183.
Feinberg et al. (2006) Nature Rev Genet 7:21-33.
Furukawa et al. (2005) Cancer Res 65:7102-7110.
Gabriel et al. (1998) Proc Natl Acad Sci USA 95:14857-14862.
Gilks et al., Mol Cell Biol 13, 1759 (1993).
Glenn et al. (1993) Hum Mol Genet 2:2001-2005.
Glenn et al. (1997) Mol Hum Reprod 3:321-332.
Goldberg et al. (2003) Hum Genet 112:334-342.
Goshu et al. (2004) Mol Endocrinol 18:1251.
Gray et al. (1999) Proc Natl Acad Sci USA 96:5616-5621.
Greally (2002) Proc Natl Acad Sci USA 99:327-332.
Greally et al. (1998) Hum Mol Genet 7:91-95.
Guo et al. (2006) J Clin Endocrinol Metab 91:4001.
Hayward et al. (1998) Proc Natl Acad Sci 95:15475-15480.
Henikoff & Henikoff (1992) 89 Proc Natl Acad Sci USA 10915-10919.
Herzing et al. (2001) Am J Hum Genet 68:1501-1505.
Higashimoto et al. (2002) Genomics 80:575-584.
Hitchins et al. (2002) Mamm Genome 13:686-691.
Hollander et al. (1998) Science 279:2118-2121.
Horike et al. (2005) Nat Genet 37:31-40.
Hovatta et al., Am J Hum Genet 65, 1114 (1999).
Hu et al. (1996) Hum Mol Genet 5:1743-1748.
Ishihara et al. (1998) Mamm Genome 9:775-777.
Jay et al. (1997) Nat Genet 17:357-361.
John et al. (2001) Dev Biol 236:387-399.
Jong et al. (1999) Hum Mol Genet 8:783-793.
Kaghad et al. (1997) Cell 90:809-819.
Kalscheuer et al. (1993) Nat Genet 5:74-78.
Kamiya et al. (2000) Hum Mol Genet 9:453-460.
Kananura et al. (2002) Am J Med Genet 114:227-229.
Karlin & Altschul (1993) 90 Proc Natl Acad Sci USA 5873-5877.
Karolchik et al. (2003) Nucleic Acids Res 31:51-54.
Kato et al. (1998) Genomics 47:146.
Kayashima et al. (2003) Hum Genet 112:220-226.
Ke et al. (2002) Mamm Genome 13:639-645.
Kelly & Locksley (2000) J Immunol 165:2982-2986.
Killian et al. (2001) Hum Mol Genet 10:1721-1728.
Kimura et al. (2004) J Hum Genet 49:273-237.
Kitsberg et al. (1993) Nature 364:459-463.
Kobayashi et al. (1997) Hum Mol Genet 6:781-786.
Kobayashi et al. (2000) Genes Cells 5:1029-1037.
Koide et al., Nat Genet 6, 9 (1994).
Krishnapuram et al. (2005) IEEE Transactions on Pattern Analysis and Machine Intelligence (PAMI) pp 957-968.
Kurosawa et al., Am J Med Genet 110, 268 (2002).
Lamb et al., J Med Genet 42, 132 (2005).
Lee et al. (1997) Nat Genet 15:181-185.
Lee et al., (2000a) Nat Genet 26:470.
Lee et al. (2000b) Hum Mol Genet 9:1813-1819.
Lee et al. (2001) Mamm Genome 12:157-162.
Lerer et al. (2005) Hum Mol Genet 14:3911-3920.
Levitsky et al. (2001) Bioinformatics 17:998-1010.
Li et al. (1993) Genomics 16:572.
Li et al. (2002) J Biol Chem 277:13518-13527.
Li et al. (2004) Proc Natl Acad Sci USA 101:7341-7346.
Lin & Floros (2002) Physiol Genomics 11:235-243.
Liu et al. (2000) J Clin Invest 106:1167-1174.
Liu et al. (2005) BMC Genet 6 Suppl 1:S160.
Liu et al., Proc Natl Acad Sci USA 99, 3717 (2002).
Luedi et al. (2005) Genome Res 15:875-884.
Luo et al. (2001) Biochim Biophys Acta 1519:216-222.
Lyle et al. (2000) Nat Genet 25:19-21.
MacDonald & Weyrick, Hum Mol Genet 6, 1873 (1997).
Maecker et al. (1998) Proc Natl Acad Sci USA 95:2458-2462.
Mager et al. (2003) Nat Genet 33:502-507.
Mai et al. (1998) Oncogene 17:1739-1741.
Mancini-Dinardo et al. (2006) Genes Dev 20:1268-1282.
Marker et al. (1995) Genomics 28:576-580.
Matsuoka et al. (1996) Proc Natl Acad Sci USA 93:3026-3030.
Mayeux et al. (2002) Am J Hum Genet 70:237.
Maynard et al. (2003) Proc Natl Acad Sci USA 100:14433.
McInnis et al. (2003) Mol Psychiatry 8:288-298.
Medhurst et al. (2001) Brain Res Mol Brain Res 86:101-114.
Meguro et al. (2001) Nat Genet 28:19-20.
Miltenberger et al. (1995) Mol Cell Biol 15:2527-2535.
Miyoshi et al. (2000) Genes Cells 5:211-220.
Mizuno et al. (2002) Biochem Biophys Res Commun 290:1499-1505.
Moens & Selleri (2006) Dev Biol 291:193-206.
Monk et al. (2006) Proc Natl Acad Sci USA 103:6623-6628.
Moore et al. (2001) Diabetes 50:199-203.
Morison et al. (2001) Nucleic Acids Res 29:275-276.
Morison et al. (2005) Trends Genet 21:457-465.
Moroy (2005) Int J Biochem Cell Biol 37:541.
Mostoslavsky et al. (2001) Nature 414:221-225.
Murphy & Jirtle (2003) Bioessays 25:577-588.
Murphy et al. (2001) Genomics 71:110-117.
Muscheck et al. (2000) Lab Invest 80:1089-1093.
Mustanski et al. (2005) Hum Genet 116:272-278.
Myers et al. (2005) Science 310:321-324.
Nabetani et al. (1997) Mol Cell Biol 17:789-798.
Nagafuchi et al. (1994) Nat Genet 8:177.
Nakabayashi et al. (2004) J Med Genet 41:601-608.
Needleman & Wunsch (1970) 48 J Mol Biol 443-453.
Niemitz & Feinberg (2004) Am J Hum Genet 74:599-609.
Nikaido et al. (2003) Genome Res 13:1402-1409.
Niu et al. (1999) Plant Mol Biol 41:1-13.
Ogawa et al. (1993a) Nature 362:749-751.
Ogawa et al. (1993b) Hum Mol Genet 2:2163-2165.
Ohlsson et al. (1993) Nat Genet 4:94-97.
Ohtsuka et al. (1985) 260 J Biol Chem 2605-2608.
Okita et al. (2003) Genomics 81:556-559.
Okutsu et al. (2000) J Biochem 127:475-483.
Ono et al. (2001) Genomics 73:232-237.
Overall et al. (1998) Mamm Genome 9:657-659.
Patel & Lazdunski (2004) Pflugers Arch 448:261-273.
Paulsen et al. (1998) Hum Mol Genet 7:1149-1159.
Paulsen et al. (2000) Hum Mol Genet 9:1829-1841.
Pearson & Lipman (1988) Proc Natl Acad Sci USA 85:2444-2448.
Pereira et al. (2003) Nat Immunol 4:464-470.
Piras et al. (2000) Mol Cell Biol 20:3308-3315.
Qian et al. (1997) Hum Mol Genet 6:2021-2029.
Rachmilewitz et al. (1992) FEBS Lett 309:25-28.
Rachmilewitz et al. (1993) Biochem Biophys Res Commun 196:659-664.
Rainier et al. (1993) Nature 362:747-749.
Ranta et al. (2000) Eur J Hum Genet 8:381-384.
Reik & Walter (2001) Nat Rev Genet 2:21-32.
Rossolini et al. (1994) 8 Mol Cell Probes 91-98.
Rougeulle et al. (1997) Nat Genet 17:14-15.
Ruf et al. (2006) Genomics 87:509-519.
Sandell et al. (2003) Proc Natl Acad Sci USA 100:4622-4627.
Sano et al. (2001) Genome Res 11:1833-1841.
Schratt et al. (2001) Mol Cell Biol 21:2933-2943.
Schweifer et al. (1997) Genomics 43:285-297.
Scott et al., Am J Hum Genet 66, 922 (2000).
Seitz et al. (2003) Nat Genet 34:261-262.
Shoichet et al. (2005) Hum Genet 117, 536 (2005).
Simonaro et al. (2006) Am J Hum Genet 78:865-870.
Singh et al. (2003) Nat Genet 33:339-341.
Smith & Waterman (1981) 2 Adv Appl Math 482-489.
Soulez et al. (1996) Mol Cell Biol 16:6065-6074.
Stefansson et al. (2002) Am J Hum Genet 71:877.
Strauch et al. (2001) Genet Epidemiol 21 Suppl 1:S204.
Strauch et al. (2005) BMC Genet 6 Suppl 1:S162.
Strichman-Almashanu et al. (2002) Genome Res 12:543-554.
Takahashi & Ko (1993) Genomics 16:161-168.
Tanamachi et al. (2001) J Exp Med 193:307-315.
Taniguchi et al. (1997) Oncogene 14:1201-1206.
Tierling et al. (2006) Genomics 87:225-235.
Umlauf et al. (2004) Nat Genet 36:1296-1300.
Van den Veyver et al. (2001) J Soc Gynecol Investig 8:305-313.
van Doonlinck et al., J Neurosci 19, RC12 (1999).
van Raamsdonk & Tilghman (2000) Development 127:5439-5448.
Vance et al. (2002) Proc Natl Acad Sci USA 99:868-873.
Vandromme et al. (1992) J Cell Biol 118:1489-1500.
Verri et al., Ann Genet 47, 281 (2004).
Vu & Hoffman (1997) Nat Genet 17:12-13.
Wakeling et al. (1998) Eur J Hum Genet 6:158-164.
Wakeling et al. (2000) J Med Genet 37:65-67.
Walter & Paulsen (2003) Hum Mol Genet 12:215-220.
Waterland & Jirtle (2003) Mol Cell Biol 23:5293-5300.
Weber et al. (2001) Mech Devel 101:133-141.
Weyrick et al. (1994) Hum Mol Genet 3:1877-1882.
Williamson et al. (1994) Genomics 22:240-242.
Williamson et al. (1995) Genet Res 65:83-93.
Witten & Frank (2005) Data mining: Practical machine learning tools and techniques (2d ed.), Morgan Kaufmann, San Francisco, United States of America.
Wood et al. (1998) Mol Cell Neurosci 11:149.
Wright et al. (2004) Development 131:5659.
Wylie et al. (2000) Genome Res 10:1711-1718.
Xin et al. (2000) J Biochem 128:847-853.
Xuan et al., Neuron 14, 1141 (1995).
Yamada et al. (2002) Gene 288:57-63.
Yamada et al. (2003) Genomics 83:402-412.
Yevtodiyenko et al. (2002) Mamm Genome 13:633-638.
Yoder et al. (1997) Trends Genet 13:335-340.
Yonan et al., Am J Hum Genet 73, 886 (2003).
Yoo & Jones (2006) Nature Rev Drug Discovery 37-50.
Yoshihashi et al. (2000) Am J Hum Genet 67:476-482.
Yu et al. (1999) Proc Natl Acad Sci 96:214-219.
Yuan et al. (1996) Hum Mol Genet 5:1931-1937.
Zara et al. (1995) Hum Mol Genet 4:1201-1207.
Zhang & Tycko (1992) Nat Genet 1:40-44.
Zhang et al. (1994) Nature 372:809-812.
Zhu et al. (2000) Gene 256:311-317.
Zimprich et al. (2001) Nat Genet 29:66-69.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for identifying an imprinted gene in a subject, the method comprising:
(a) providing a first data set comprising a plurality of nucleic acid sequences, wherein the nucleic acid sequences comprise genomic DNA sequences corresponding to a plurality of genes known to be imprinted in the subject;
(b) providing a second data set comprising a plurality of nucleic acid sequences, wherein the nucleic acid sequences comprise genomic DNA sequences corresponding to a plurality of genes known not to be imprinted in the subject;
(c) identifying one or more features that by themselves or in combination are differentially present or absent from the first data set as compared to the second data set; and
(d) applying the one or more features to a test data set comprising a plurality of genomic DNA sequences which correspond to one or more genes for which the imprinting status is unknown to thereby identify an imprinted gene in a subject.

2. The method of claim 1, wherein the subject is a human.

3. The method of claim 1, wherein the genomic DNA sequences include untranslated sequences of at least 1 kilobase, 2 kilobases, 5 kilobases, 10 kilobases, 25 kilobases, 50 kilobases, 100 kilobases, or greater than 100 kilobases for one or more of the plurality of genes known to be imprinted in the subject, one or more of the plurality of genes known not to be imprinted in the subject, and combinations thereof.

4. The method of claim 3, wherein the genomic DNA sequences comprise 5′ untranslated sequences, 3′ untranslated sequences, or both 5′ and 3′ untranslated sequences.

5. The method of claim 1, wherein the features are selected from those set forth in Table 4.

6. The method of claim 1, wherein the identifying comprises training an algorithm using the first data set as a first training data set and the second data set as a second training data set to thereby identify one or more features in the first and second data sets that are predictive of imprinting status.

7. A method for identifying a feature in a subject with respect to an imprinted gene, the method comprising:
(a) obtaining a biological sample from the subject, wherein the biological sample comprises one or more nucleic acid molecules derived from one or more of the genes listed in Table 1; and
(b) analyzing the one or more nucleic acid molecules,
whereby a feature is identified in the subject with respect to the imprinted gene.

8. The method of claim 7, wherein the feature is selected from the group consisting of a genetic feature, an epigenomic feature, and combinations thereof.

9. The method of claim 8, wherein the genetic feature comprises a genotype of the subject with respect to at least one gene listed in Table 1.

10. The method of claim 8, wherein the epigenomic feature is selected from the group consisting of a DNA sequence modification (such as methylation), a nucleosome positioning feature, a chromatin state, and a histone modification (such as methylation or acetylation or similar).

11. The method of claim 7, wherein the biological sample comprises genomic DNA from the subject.

12. The method of claim 7, wherein the analyzing comprises sequencing at least a portion of the one or more nucleic acid molecules derived from one or more of the genes listed in Table 1.

13. The method of claim 12, wherein the subject is heterozygous for one or more polymorphisms located in the portion of the one or more nucleic acid molecules derived from one or more of the genes listed in Table 1, and the sequencing identifies the one or more polymorphisms.

14. The method of claim 7, wherein the method further comprises screening a biological sample from one or both biological parents of the subject to identify which parent transmitted each allele to the subject.

15. The method of claim 14, further comprising predicting whether or not one or more of the alleles is likely to be expressed in the subject.

16. The method of claim 15, wherein the predicting comprises correlating maternal or paternal inheritance of the one or more alleles with an assessment of whether the one or more alleles is expressed when inherited maternally or paternally.

17. A method for detecting a presence of or a susceptibility to a medical condition associated with parent-of-origin dependent monoallelic expression in a subject, the method comprising:
(a) obtaining a biological sample from the subject, wherein the biological sample comprises one or more nucleic acid molecules;
(b) analyzing the one or more nucleic acid molecules for a feature with respect to parent-of-origin for one or both alleles of at least one imprinted gene; and
(c) determining whether the feature correlates with a presence of or a susceptibility to a medical condition associated with monoallelic expression, whereby a presence of or a susceptibility to a medical condition associated with parent-of-origin dependent monoallelic expression in the subject is detected.

18. The method of claim 17, wherein the feature is selected from the group consisting of a genetic feature, an epigenomic feature, and combinations thereof.

19. The method of claim 18, wherein the genetic feature comprises a genotype of the subject with respect to at least one gene listed in Table 1.

20. The method of claim 18, wherein the epigenomic feature is selected from the group consisting of a DNA sequence methylation state, a nucleosome positioning feature, and a histone modification.

21. The method of claim 17, wherein the feature relates to a gene listed in Table 1 the expression or lack of expression of which is associated with a medical condition.

22. The method of claim 17, wherein the medical condition is selected from the group consisting of alcoholism, Alzheimer's disease, asthma/atopy, autism, bipolar disorder, obesity, diabetes, Parental Uniparental Disomy (UPD), cancer, epilepsy, DiGeorge syndrome, and schizophrenia.

23. The method of claim 17, wherein the at least one imprinted gene is selected from DLGAP2 and KCNK9.
